(19) United States
(12) Patent Application Publication
(10) Pub. No.: US 2022/0218670 A1
(43) Pub. Date: Jul. 14, 2022

(54) METHODS OF TREATING DISEASES CHARACTERISED BY VASOCONSTRICTION

(71) Applicant: Gesynta Pharma AB, Solna (SE)

(72) Inventor: Per-Johan Jakobsson, Bromma (SE)

(21) Appl. No.: 17/569,049

(22) Filed: Jan. 5, 2022

Related U.S. Application Data
(63) Continuation of application No. 16/079,203, filed on Aug. 23, 2018, now abandoned.

(30) Foreign Application Priority Data
Feb. 25, 2016 (GB) 1603311.0

(51) Int. Cl. A61K 31/4184; A61K 31/4375; A61K 45/00; A61P 9/08; A61K 31/4439; A61K 31/454

(52) U.S. Cl. CPC A61K 31/4184; A61K 31/4375; A61K 45/00; A61K 45/06; A61K 31/4439; A61K 31/454; A61P 9/08

(57) ABSTRACT

There is herein provided a compound that is an mPGES-1 inhibitor, or a prodrug thereof, for use in the treatment or prophylaxis of a disease or disorder characterised by vasoconstriction.

### FIELD OF THE INVENTION

The present invention relates to the use of compounds that are inhibitors of the enzyme microsomal prostaglandin E₂ synthase-1 (mPGES-1) in the treatment of diseases and disorders characterised by vasoconstriction, and to methods of treatment of such diseases and disorders based upon such a use. In particular, the present invention relates to the treatment of diseases and disorders characterised by vasoconstriction associated with inflammation.

### BACKGROUND OF THE INVENTION

The listing or discussion of an apparently prior-published document in this specification should not necessarily be taken as an acknowledgement that the document is part of the state of the art or is common general knowledge.

There are numerous diseases and disorders that are characterised by (i.e. have as a causative component) a narrowing of the blood vessels resulting from contraction of the muscular wall of those vessels, in particular the large arteries and small arterioles, which is known as vasoconstriction. Such vessels may be more prone to contract via contractions of activated or hyper excitable smooth muscles surrounding the vessels or by processes of inflammation, causing oedema in the vessel walls and eventually fibrosis that renders the vessels and surrounding tissues stiffer.

Such conditions may be the direct result of the effects of the narrowing of the blood vessels or may be indirectly related through the effect of such narrowing on the operation of other bodily processes. For example, vasoconstriction in the small blood vessels may lead to a loss of blood circulation to the extremities, whereas vasoconstriction in the lung vasculature may lead to increased stress on the circulatory system resulting in pulmonary arterial hypertension.

Vasoconstriction itself may be triggered by a number of factors, including inflammatory conditions, notable among which is the systemic autoimmune disease scleroderma, which manifests itself as a hardening of the skin, characterised by initial inflammation followed by fibrosis and thickening and which, in its more severe form, may also affect internal organs (such as the lung tissue and pulmonary circulation). This type of blood vessel narrowing is very different to that observed in atherosclerosis, which results from a thickening of the arterial wall as a result of invasion and accumulation of white blood cells, proliferation of intimal smooth muscle cell and lipid deposits, thus restricting blood flow through the formation of fibrous fatty plaques.

Conditions such as those associated with scleroderma (and therefore characterised by vasocontraction) have been treated with nonsteroidal anti-inflammatory drugs (NSAIDs) with the intention of treating the underlying inflammation. However, such NSAIDs have been found to have secondary effects which may serve to prolong or worsen the degree of vasoconstriction.

NSAIDs are amongst the world's most used and recognisable medications with billions of doses prescribed each year to treat inflammation, pain and fever. NSAIDs include traditional forms, such as ibuprofen and diclofenac, as well as selective inhibitors of COX-2, such as celecoxib (Celebrex™).

NSAIDs and COX-2 inhibitors reduce inflammation through inhibition of one or both isoforms of COX enzymes. The cyclooxygenase (COX) enzyme exists in two forms; one that is constitutively expressed in many cells and tissues (COX-1) and one that in most cells and tissues is induced by pro-inflammatory stimuli, such as cytokines, during an inflammatory response (COX-2).

COXs metabolise arachidonic acid to the unstable intermediate prostaglandin H₂ (PGH₂), which is further metabolized to other prostaglandins including PGE₂, PGF₂a, PGD₂, prostacyclin and thromboxane A₂. These arachidonic acid metabolites are known to have pronounced physiological and pathophysiological activity including pro-inflammatory effects. PGE₂ in particular is known to be a strong pro-inflammatory mediator, and is also known to induce fever, inflammation and pain. Consequently, numerous drugs were developed with a view to inhibiting the formation of PGE₂, predominantly by inhibition of COX-1 and/or COX-2.

Recently, concerns over cardiovascular side effects associated with the use of these drugs has resulted in a series of regulatory events including (i) the withdrawal of the blockbuster drug Vioxx in 2004, (ii) the introduction of ‘black box’ warnings on some NSAIDs from 2005 and on all drugs in this class since 2015, (iii) the withdrawal of Onsenal (celecoxib) for the prevention of cancer in 2011 and (iv) the reclassification of the over-the-counter medication diclofenac as prescription only in 2015 (UK). Now the fear of cardiovascular events caused by NSAIDs has become a public health issue resulting in the cautious prescribing of COX-2 selective drugs in favor of older style medication which are more toxic to the gut and a failure to realize the full clinical potential of NSAIDs in the prevention of cancer (Scarpignato, C. et al., BMC Med., 13, 55 (2015); Garcia Rodriguez, L. A., et al., Recent Results Cancer Res., 191, 67-93 (2013)).

Moreover, the inhibition of COXs has the disadvantage that it results in the reduction of the formation of all metabolites downstream of PGH₂, some of which are known to have beneficial properties. For example, it has been shown that there is a link between inhibition of COX-2 and the endothelial nitric oxide synthase (eNOS) (Ahmetaj-Shala, B. et al., Circulation, 131, 633-642 (2015); Yu, Y. et al., Sci Transl Med., 4, 132-154 (2012); Ahmetaj-Shala, B. et al, Circulation (2014)). This effect results in decreased levels of nitric oxide (NO), which is a known vasorelaxation agent and is therefore capable of counteracting the vasoconstriction and effects associated therewith.

An alternative treatment of inflammatory diseases that does not give rise to the above-mentioned negative effects would thus be of real benefit in the clinic.

Various approaches towards the provision of alternative treatments for inflammation have focused on selectively inhibiting the formation of protaglandins via the arachidonic acid pathway. In one such approach, it has been proposed to utilize a drug that selectively inhibits the transformation of PGH₂ to the pro-inflammatory mediator PGE₂. It has been shown that such an effect can be achieved by inhibition of mPGES-1.

Inhibition of mPGES-1 is a well-developed area of preclinical research with studies showing that its genetic deletion protects against inflammation, pain and cancer (Friesen, R. W. and Mancini, J. A., J Med Chem., 51, 4059-4067 (2008); Howe, L. R. et al., Prostaglandins Other Lipid Mediat., 106, 99-105 (2013); Korotkova, M. and Jakobsson P. J., Basic Clin Pharmacol Toxicol., 114, 64-69 (2014)). However, the lack of a complete understanding surrounding cardiovascular toxicity and other negative effects has meant that research and translation of mPGES-1 as a therapeutic target to replace COX-2 in the treatment of a wide range of diseases and disorders has been restricted.

### DESCRIPTION OF THE INVENTION

We have now surprisingly found that, unlike inhibition of COX-2, inhibition of mPGES-1 can occur without inhibition of endothelial nitric oxide synthase (eNOS). Thus, compounds that are inhibitors of mPGES-1 may be unexpectedly effective in the treatment of diseases and disorders characterised by vasoconstriction, particularly those diseases and disorders associated with inflammation.

In a first aspect of the invention, there is provided a compound that is an mPGES-1 inhibitor, or a prodrug thereof, for use in the treatment or prophylaxis of a disease or disorder characterised by vasoconstriction.

In an alternative first aspect of the invention, there is provided the use of a compound that is an mPGES-1 inhibitor, or a prodrug thereof, in the manufacture of a medicament for use in the treatment or prophylaxis of a disease or disorder characterised by vasoconstriction.

In a further alternative first aspect of the invention, there is provided a method of treating or preventing a disease or disorder characterised by vasoconstriction comprising administering to a patient in need thereof an effective amount of a compound that is an mPGES-1 inhibitor, or a prodrug thereof.

The skilled person will understand that references herein to particular aspects of the invention will include references to all embodiments and particular features thereof.

Moreover, all embodiments of particular aspects of the invention may be combined with one or more other embodiments of that aspect of the invention to form further embodiments without departing from the teaching of the invention.

Unless indicated otherwise, all technical and scientific terms used herein have the same meaning as commonly understood by one of ordinary skill in the art to which this invention pertains.

The skilled person will understand that compounds that are mPGES-1 inhibitors may be provided in the form of pharmaceutically acceptable salts.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, the skilled person will understand that compounds that are mPGES-1 inhibitors may exist as solids, and thus the scope of the invention includes all amorphous, crystalline and part crystalline forms thereof, and may also exist as oils. Where such compounds exist in crystalline and part crystalline forms, such forms may include solvates, which are included in the scope of the invention. Compounds that are mPGES-1 inhibitors may also exist in solution.

Compounds as disclosed herein may contain double bonds and may thus exist as E (entgegen) and Z (zusammen) geometric isomers about each individual double bond. All such isomers and mixtures thereof are included within the scope of the invention.

Compounds as disclosed herein may also exhibit tautomerism. All tautomeric forms and mixtures thereof are included within the scope of the invention.

Compounds as disclosed herein may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

The skilled person will understand that references to particular diseases and disorders will refer to particular medical conditions as described herein. Thus, the terms disease and disorder may be used interchangeably or may be replaced with references to medical conditions.

The skilled person will understand that references to the treatment of a particular condition (or, similarly, to treating that condition) take their normal meaning in the field of medicine. In particular, the term may refer to achieving a reduction in the severity of one or more clinical symptom associated with the condition. For example, in the treatment of conditions characterised by vasoconstriction, the term may refer to achieving a reduction of the degree of vasoconstriction (e.g. such that blood flow is increased and/or blood pressure is decreased), and may also refer to achieving a reduction in associated symptoms, such as inflammation. Such reductions may be measured using objective analysis (e.g. the measurement of blood flow and/or blood pressure) and/or through subjective analysis (for example, by assessment through examination by a physician).

The skilled person will understand that references to the prophylaxis or prevention of a particular condition take their normal meaning in the field of medicine, and that these terms are synonymous and may be used interchangeably. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

As used herein, references to patients will refer to a living subject being treated, including mammalian (e.g. human) patients.

As used herein, the term effective amount will refer to an amount of a compound that confers a therapeutic effect on the treated patient. The effect may be objective (i.e. measurable by some test or marker) or subjective (i.e. the subject gives an indication of and/or feels an effect). The skilled person will be able to determine a suitable therapeutically acceptable dose using techniques that are routine in the art.

The skilled person will understand that the references to an mPGES-1 inhibitor will refer to chemical compounds having sufficient activity as inhibitors of the enzyme mPGES-1 to produce a therapeutic effect (i.e. an effect in treatment or prophylaxis as described herein).

The skilled person will be able to assess the ability of compounds to act as mPGES-1 inhibitors using screens and assays (e.g. in vitro assays) based on techniques that are well known to those skilled in the art, e.g. through measurement of the production of prostaglandin (PG) E₂ (such as in the assay described in the PCT application published as WO 2010/100249, the contents of which are incorporated herein by reference).

In particular, such compounds that are mPGES-1 inhibitors may have experimentally measurable activity in the inhibition of mPGES-1 resulting in an IC₅₀ of lower than 10 μM (e.g. lower than 5 μM, particularly lower than 1 μM, such as lower than 50 nM, for example lower than 10 nM).

Further, such compounds that are mPGES-1 inhibitors may have experimentally measurable activity in the inhibition of mPGES-1 at a concentration of 10 μM (and in a suitable medium, e.g. a suitable reconstitution buffer) of at least 50% (e.g. at least 60%, particularly at least 70%, for example at least 80%, such as at least 90%).

The skilled person will also understand that compounds that are referred to herein as mPGES-1 inhibitors may also be selective mPGES-1 inhibitors, which term will be understood by those skilled in the art. In particular, the term may indicate that such compounds are able to inhibit mPGES-1 without causing significant (i.e. therapeutically relevant) levels of inhibition of other enzymes, such as enzymes functioning as part of the arachidonic acid pathway (e.g. without affecting the production of PGH₂).

The skilled person will understand that the present invention may utilise not only compounds that possess the required pharmacological activity (i.e. as mPGES-1 inhibitors) as such, but also compounds which may not possess such activity but may be administered parenterally or orally and thereafter be metabolised in the body to form compounds processing the required activity (or may possess some pharmacological activity, but wherein such activity is appreciably lower than that of the active compounds to which they are metabolised). Such compounds may therefore be described as prodrugs of compounds of the invention.

Thus, as used herein, references to prodrugs of mPGES-1 inhibitors will include compounds that form an mPGES-1 inhibitor, in an experimentally-detectable amount and within a predetermined time (e.g. within 2 hours), following enteral or parenteral administration (e.g. oral or parenteral administration).

As described herein, the skilled person will be able to identify compounds that are mPGES-1 inhibitors, or prodrugs thereof, using techniques that are routine in the art. Moreover, the skilled person will be aware of numerous compounds that are disclosed as mPGES-1 inhibitors, which compounds will be suitable for use in the present invention.

For example, the present invention may utilise as the required mPGES-1 inhibitor a compound as described as being an mPGES-1 inhibitor, or a pharmaceutically acceptable salt thereof (or, in the case where the compound is described as being in salt form, the corresponding non-salt form or a different pharmaceutically acceptable salt form thereof) or a prodrug thereof, in any of the following publications (the contents of which are incorporated herein by reference, including in particular the compounds as exemplified therein):

WO 2004/028458

WO 2005/024059

WO 20061063466

WO 2006/077366

WO 2006/077401

WO 2007/059611

WO 2007/061853

WO 2007/095753

WO 2007/104019

WO 2007/134434

WO 2008/071173

WO 2009/117985

WO 2010/034796

WO 2010/034797

WO 2010/034798

WO 2010/034799

WO 2010/100249

WO 2010/127152

WO 2011/023812

WO 2011/048004

WO 2011/077313

WO 2012/022792

WO 2012/022793

WO 2012/055995

WO 2012/076672

WO 2012/076673

WO 2012/076674

WO 2012/110860

WO 2012/117062

WO 2013/024898

WO 2013/038308

WO 2013/072825

WO 2013/118071

WO 2013/153535

WO 2013/186692

WO 2014/167444

WO 2014/204370

WO 2015/059618

WO 2015/125842

WO 2015/158204

WO 2016/016861

For the avoidance of doubt, references herein to disclosures provided in patent publications will refer to those discloses in their original form, i.e. as provided in the relevant A1 or A2 publication, as appropriate (but taking account of any accepted corrections thereof).

In particular, the present invention may utilise as the required mPGES-1 inhibitor a compound as described as being an mPGES-1 inhibitor, or a pharmaceutically acceptable salt thereof (or, in the case where the compound is described as being in salt form, the corresponding non-salt form or a different pharmaceutically acceptable salt form thereof) or a prodrug thereof, in any of the following publications (the contents of which are incorporated herein by references, including in particular the compounds as exemplified therein):
(a) WO 2012/117062 and/or WO 2011/023812; and/or
(b) WO 2010/100249 and/or WO 2012/022793.

In particular embodiments, compounds that may be utilised as the required mPGES-1 inhibitor may include the following compounds as numbered in Group A below, or a pharmaceutically acceptable salt thereof:

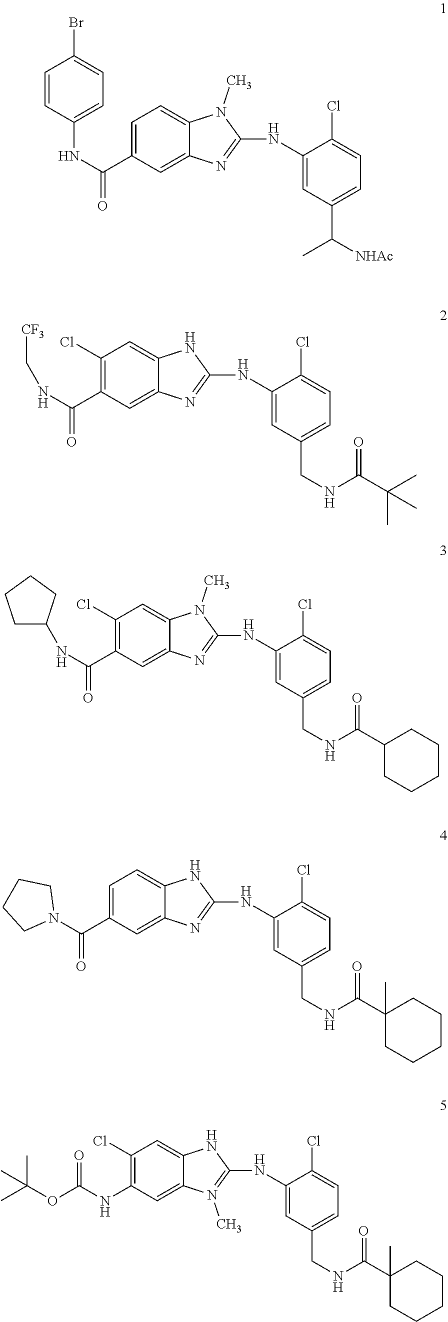

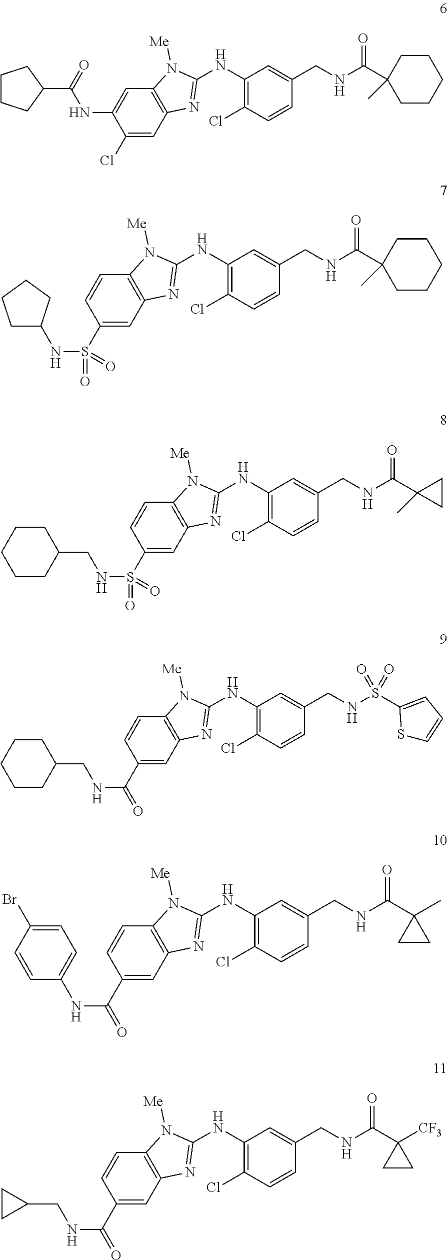 [SMILES: Cn1c(Nc2cc(CNC(=O)C3(C)CCCCC3)ccc2Cl)nc2cc(Cl)c(NC(=O)C3CCCC3)cc21 ; Cn1c(Nc2cc(CNC(=O)C3(C)CCCCC3)ccc2Cl)nc2cc(S(=O)(=O)NC3CCCC3)ccc21 ; Cn1c(Nc2cc(CNC(=O)C3(C)CC3)ccc2Cl)nc2cc(S(=O)(=O)NCC3CCCCC3)ccc21 ; Cn1c(Nc2cc(CNS(=O)(=O)c3cccs3)ccc2Cl)nc2cc(C(=O)NCC3CCCCC3)ccc21 ; Cn1c(Nc2cc(CNC(=O)C3(C)CC3)ccc2Cl)nc2cc(C(=O)Nc3ccc(Br)cc3)ccc21 ; Cn1c(Nc2cc(CNC(=O)C3(C(F)(F)F)CC3)ccc2Cl)nc2cc(C(=O)NCC3CC3)ccc21]

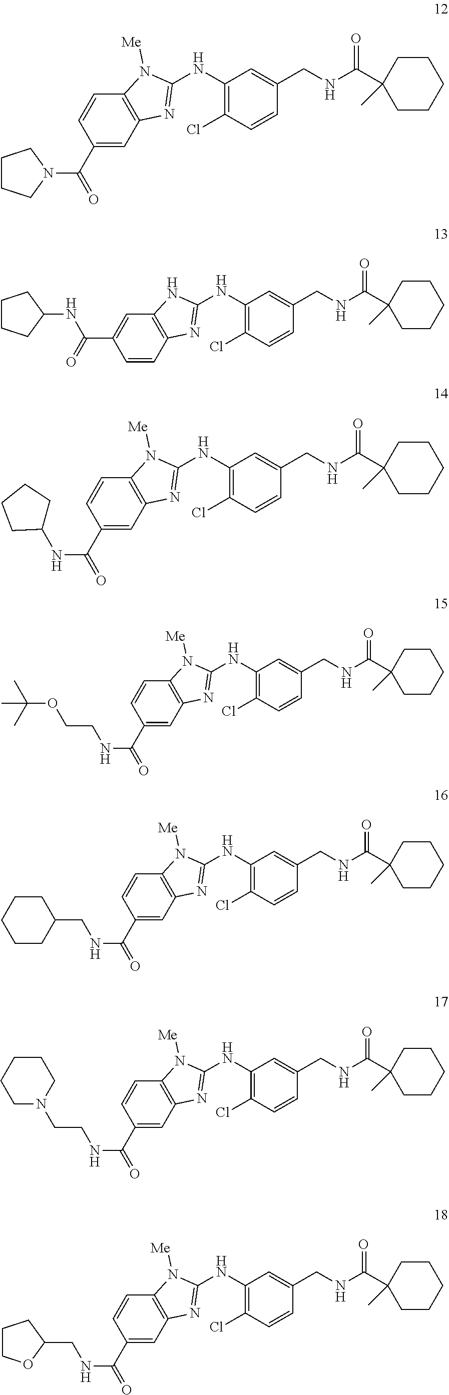

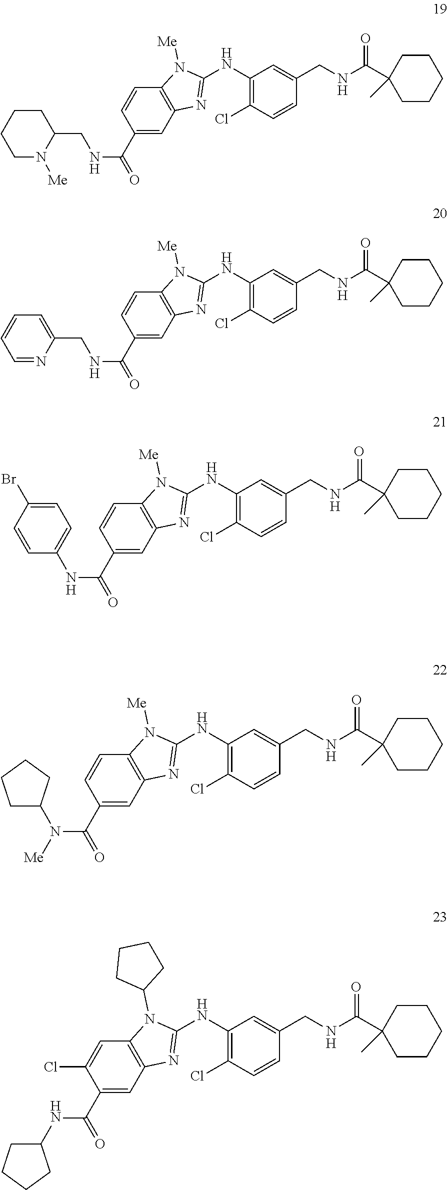 [SMILES: CN1CCCCC1CNC(=O)c1ccc2c(c1)nc(Nc1cc(CNC(=O)C3(C)CCCCC3)ccc1Cl)n2C ; Cn1c(Nc2cc(CNC(=O)C3(C)CCCCC3)ccc2Cl)nc2cc(C(=O)NCc3ccccn3)ccc21 ; Cn1c(Nc2cc(CNC(=O)C3(C)CCCCC3)ccc2Cl)nc2cc(C(=O)Nc3ccc(Br)cc3)ccc21 ; CN(C(=O)c1ccc2c(c1)nc(Nc1cc(CNC(=O)C3(C)CCCCC3)ccc1Cl)n2C)C1CCCC1 ; CC1(C(=O)NCc2ccc(Cl)c(Nc3nc4cc(C(=O)NC5CCCC5)c(Cl)cc4n3C3CCCC3)c2)CCCCC1]

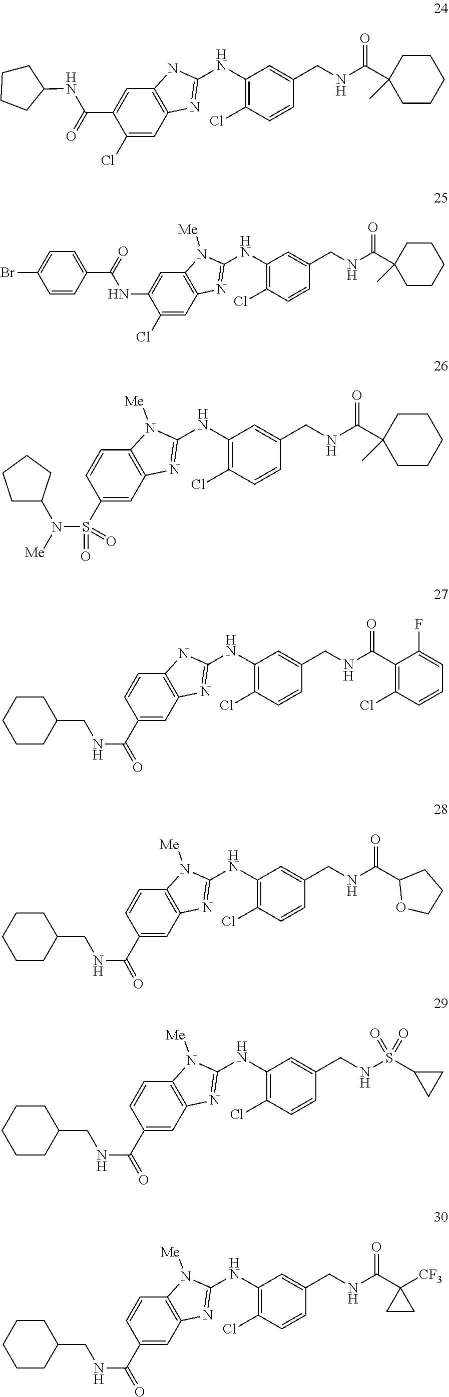

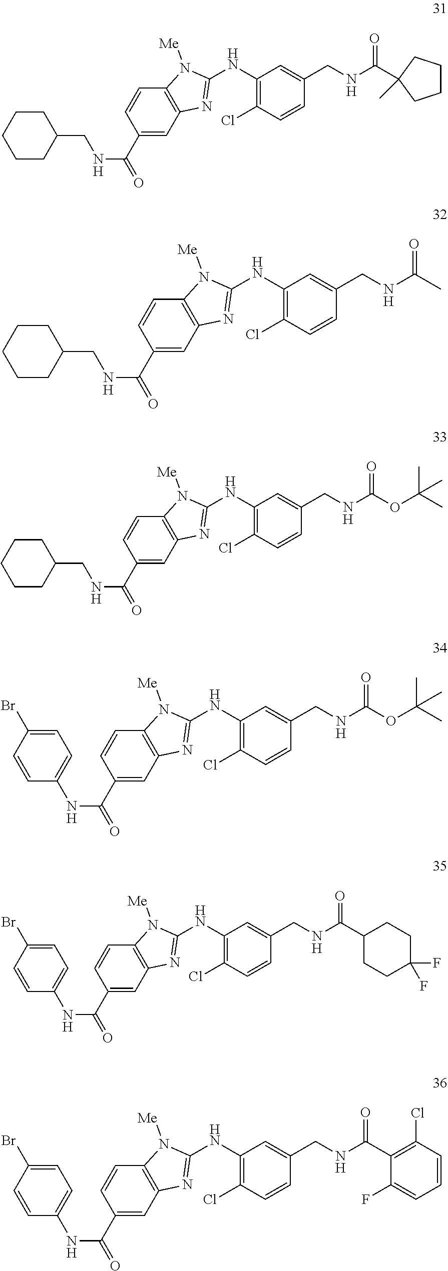 [SMILES: Cn1c(Nc2cc(CNC(=O)C3(C)CCCC3)ccc2Cl)nc2cc(C(=O)NCC3CCCCC3)ccc21 ; CC(=O)NCc1ccc(Cl)c(Nc2nc3cc(C(=O)NCC4CCCCC4)ccc3n2C)c1 ; Cn1c(Nc2cc(CNC(=O)OC(C)(C)C)ccc2Cl)nc2cc(C(=O)NCC3CCCCC3)ccc21 ; Cn1c(Nc2cc(CNC(=O)OC(C)(C)C)ccc2Cl)nc2cc(C(=O)Nc3ccc(Br)cc3)ccc21 ; Cn1c(Nc2cc(CNC(=O)C3CCC(F)(F)CC3)ccc2Cl)nc2cc(C(=O)Nc3ccc(Br)cc3)ccc21 ; Cn1c(Nc2cc(CNC(=O)c3c(F)cccc3Cl)ccc2Cl)nc2cc(C(=O)Nc3ccc(Br)cc3)ccc21]

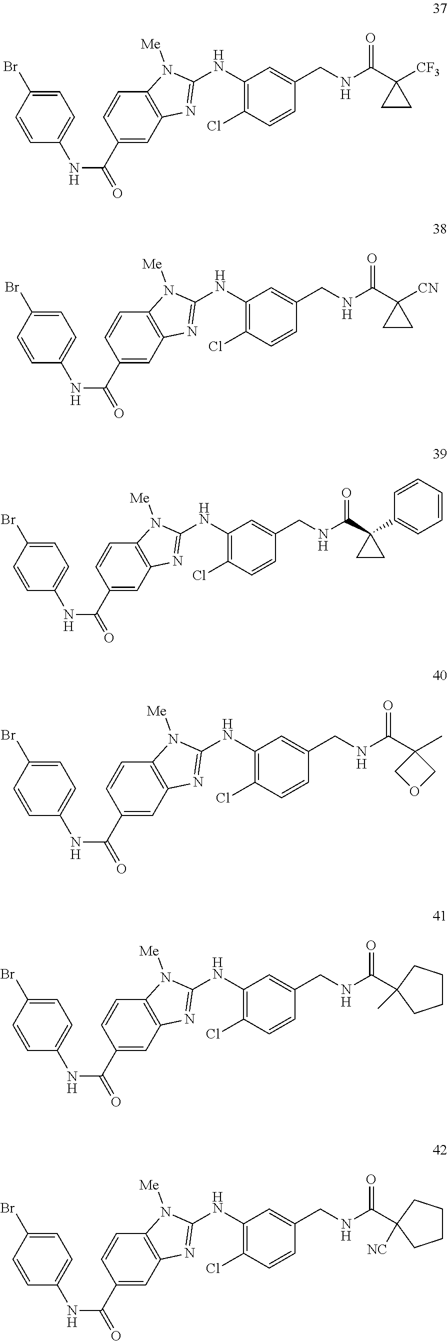

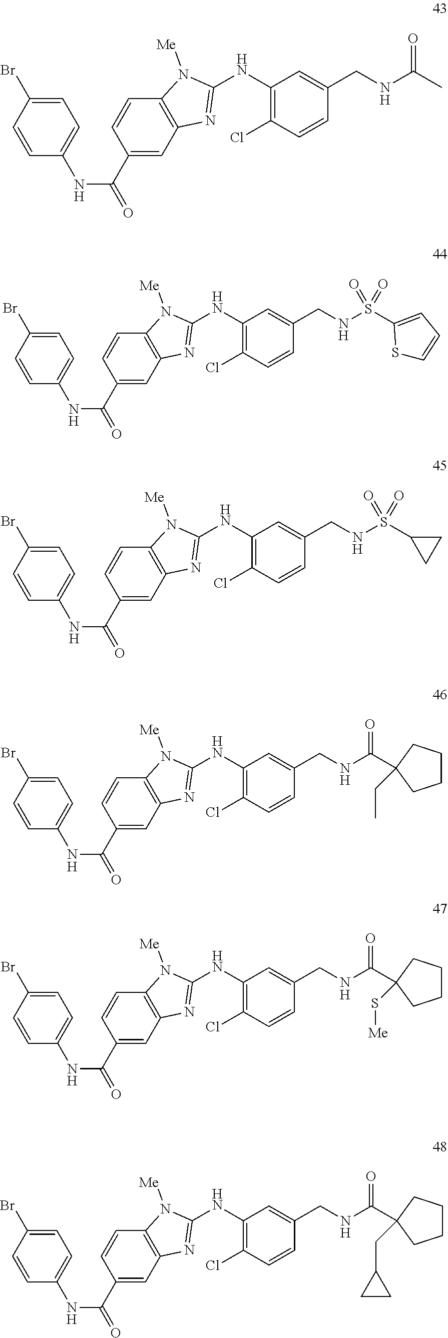 [SMILES: CC(=O)NCc1ccc(Cl)c(Nc2nc3cc(C(=O)Nc4ccc(Br)cc4)ccc3n2C)c1 ; Cn1c(Nc2cc(CNS(=O)(=O)c3cccs3)ccc2Cl)nc2cc(C(=O)Nc3ccc(Br)cc3)ccc21 ; Cn1c(Nc2cc(CNS(=O)(=O)C3CC3)ccc2Cl)nc2cc(C(=O)Nc3ccc(Br)cc3)ccc21 ; CCC1(C(=O)NCc2ccc(Cl)c(Nc3nc4cc(C(=O)Nc5ccc(Br)cc5)ccc4n3C)c2)CCCC1 ; CSC1(C(=O)NCc2ccc(Cl)c(Nc3nc4cc(C(=O)Nc5ccc(Br)cc5)ccc4n3C)c2)CCCC1 ; Cn1c(Nc2cc(CNC(=O)C3(CC4CC4)CCCC3)ccc2Cl)nc2cc(C(=O)Nc3ccc(Br)cc3)ccc21]

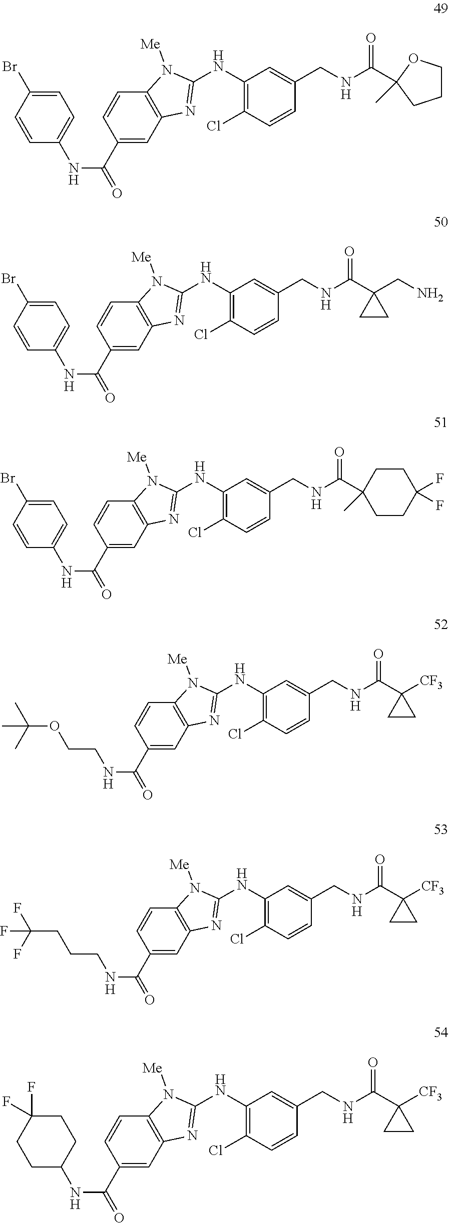 [SMILES: Cn1c(Nc2cc(CNC(=O)C3(C)CCCO3)ccc2Cl)nc2cc(C(=O)Nc3ccc(Br)cc3)ccc21 ; Cn1c(Nc2cc(CNC(=O)C3(CN)CC3)ccc2Cl)nc2cc(C(=O)Nc3ccc(Br)cc3)ccc21 ; Cn1c(Nc2cc(CNC(=O)C3(C)CCC(F)(F)CC3)ccc2Cl)nc2cc(C(=O)Nc3ccc(Br)cc3)ccc21 ; Cn1c(Nc2cc(CNC(=O)C3(C(F)(F)F)CC3)ccc2Cl)nc2cc(C(=O)NCCOC(C)(C)C)ccc21 ; Cn1c(Nc2cc(CNC(=O)C3(C(F)(F)F)CC3)ccc2Cl)nc2cc(C(=O)NCCCC(F)(F)F)ccc21 ; Cn1c(Nc2cc(CNC(=O)C3(C(F)(F)F)CC3)ccc2Cl)nc2cc(C(=O)NC3CCC(F)(F)CC3)ccc21]

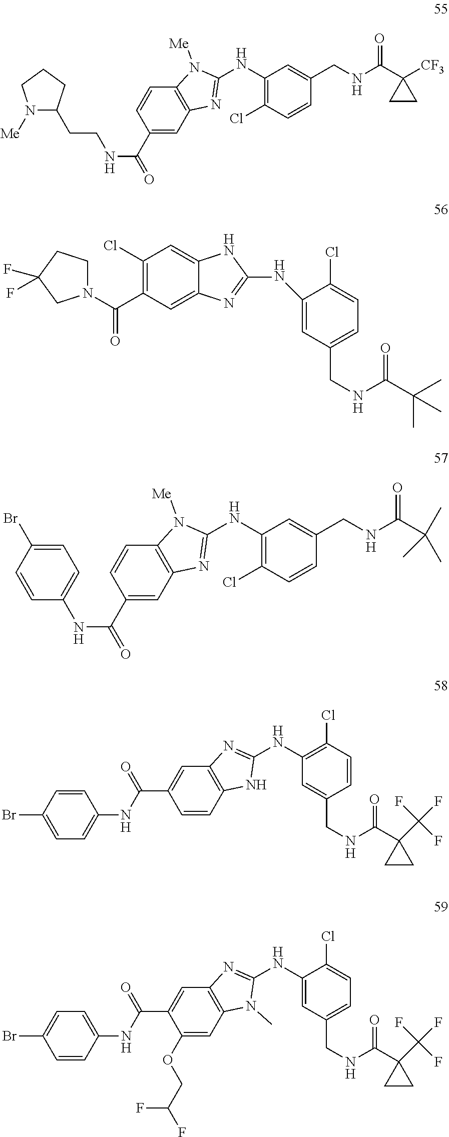 [SMILES: CN1CCCC1CCNC(=O)c1ccc2c(c1)nc(Nc1cc(CNC(=O)C3(C(F)(F)F)CC3)ccc1Cl)n2C ; CC(C)(C)C(=O)NCc1ccc(Cl)c(Nc2nc3cc(C(=O)N4CCC(F)(F)C4)c(Cl)cc3[nH]2)c1 ; Cn1c(Nc2cc(CNC(=O)C(C)(C)C)ccc2Cl)nc2cc(C(=O)Nc3ccc(Br)cc3)ccc21 ; O=C(Nc1ccc(Br)cc1)c1ccc2[nH]c(Nc3cc(CNC(=O)C4(C(F)(F)F)CC4)ccc3Cl)nc2c1 ; Cn1c(Nc2cc(CNC(=O)C3(C(F)(F)F)CC3)ccc2Cl)nc2cc(C(=O)Nc3ccc(Br)cc3)c(OCC(F)F)cc21]

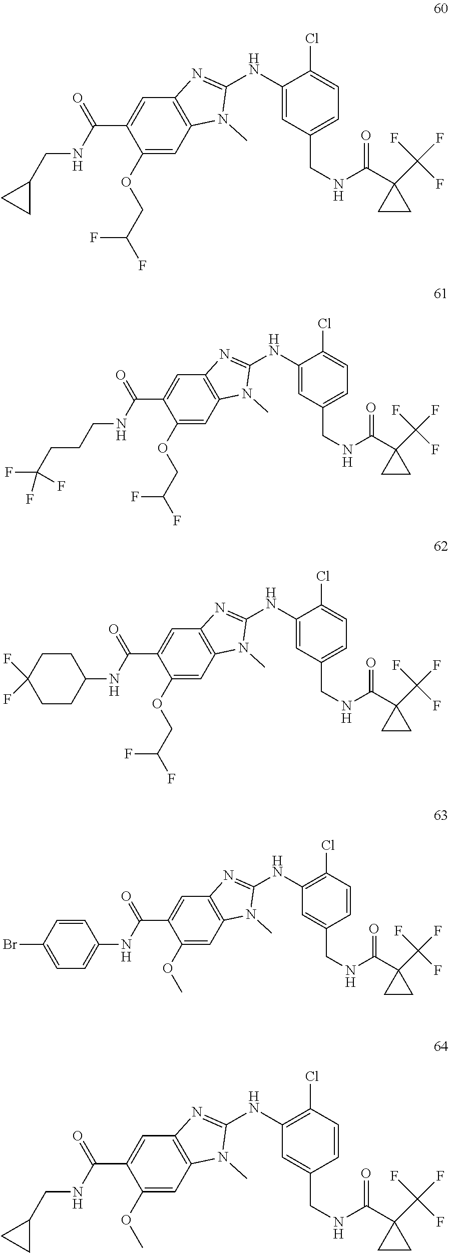 [SMILES: Cn1c(Nc2cc(CNC(=O)C3(C(F)(F)F)CC3)ccc2Cl)nc2cc(C(=O)NCC3CC3)c(OCC(F)F)cc21 ; Cn1c(Nc2cc(CNC(=O)C3(C(F)(F)F)CC3)ccc2Cl)nc2cc(C(=O)NCCCC(F)(F)F)c(OCC(F)F)cc21 ; Cn1c(Nc2cc(CNC(=O)C3(C(F)(F)F)CC3)ccc2Cl)nc2cc(C(=O)NC3CCC(F)(F)CC3)c(OCC(F)F)cc21 ; COc1cc2c(cc1C(=O)Nc1ccc(Br)cc1)nc(Nc1cc(CNC(=O)C3(C(F)(F)F)CC3)ccc1Cl)n2C ; COc1cc2c(cc1C(=O)NCC1CC1)nc(Nc1cc(CNC(=O)C3(C(F)(F)F)CC3)ccc1Cl)n2C]

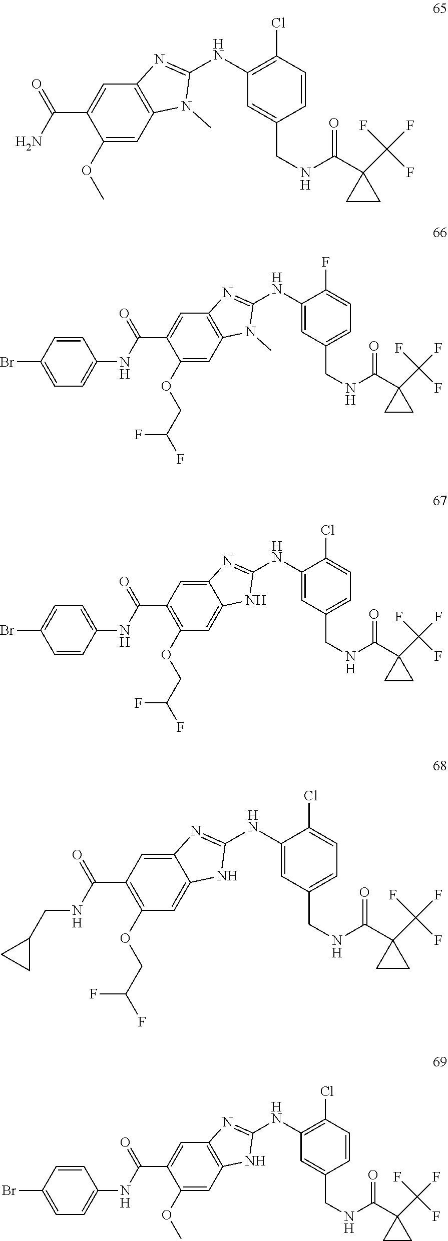 [SMILES: COc1cc2c(cc1C(N)=O)nc(Nc1cc(CNC(=O)C3(C(F)(F)F)CC3)ccc1Cl)n2C ; Cn1c(Nc2cc(CNC(=O)C3(C(F)(F)F)CC3)ccc2F)nc2cc(C(=O)Nc3ccc(Br)cc3)c(OCC(F)F)cc21 ; O=C(Nc1ccc(Br)cc1)c1cc2nc(Nc3cc(CNC(=O)C4(C(F)(F)F)CC4)ccc3Cl)[nH]c2cc1OCC(F)F ; O=C(NCC1CC1)c1cc2nc(Nc3cc(CNC(=O)C4(C(F)(F)F)CC4)ccc3Cl)[nH]c2cc1OCC(F)F ; COc1cc2[nH]c(Nc3cc(CNC(=O)C4(C(F)(F)F)CC4)ccc3Cl)nc2cc1C(=O)Nc1ccc(Br)cc1]

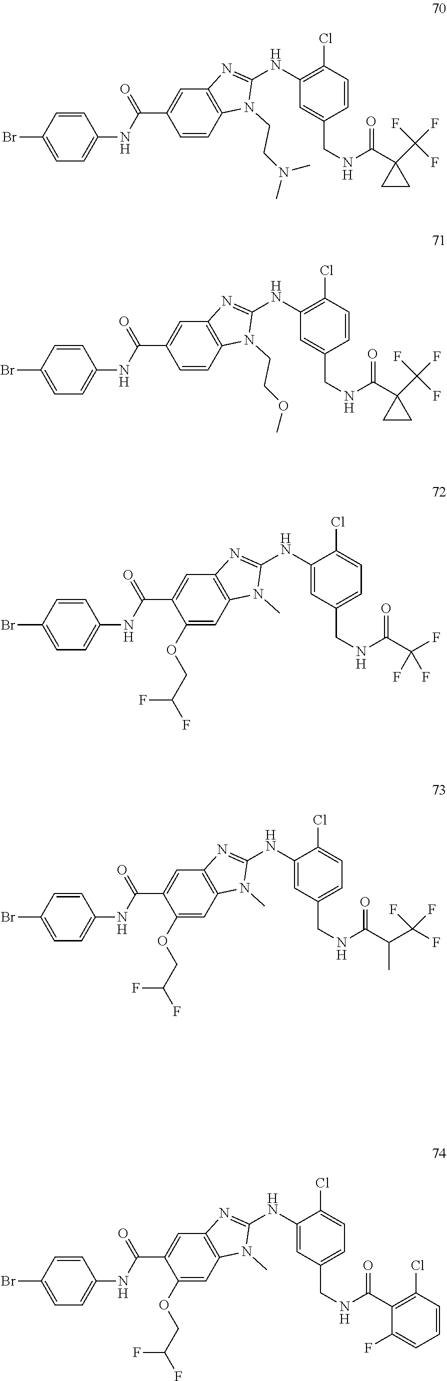 [SMILES: CN(C)CCn1c(Nc2cc(CNC(=O)C3(C(F)(F)F)CC3)ccc2Cl)nc2cc(C(=O)Nc3ccc(Br)cc3)ccc21 ; COCCn1c(Nc2cc(CNC(=O)C3(C(F)(F)F)CC3)ccc2Cl)nc2cc(C(=O)Nc3ccc(Br)cc3)ccc21 ; Cn1c(Nc2cc(CNC(=O)C(F)(F)F)ccc2Cl)nc2cc(C(=O)Nc3ccc(Br)cc3)c(OCC(F)F)cc21 ; CC(C(=O)NCc1ccc(Cl)c(Nc2nc3cc(C(=O)Nc4ccc(Br)cc4)c(OCC(F)F)cc3n2C)c1)C(F)(F)F ; Cn1c(Nc2cc(CNC(=O)c3c(F)cccc3Cl)ccc2Cl)nc2cc(C(=O)Nc3ccc(Br)cc3)c(OCC(F)F)cc21]

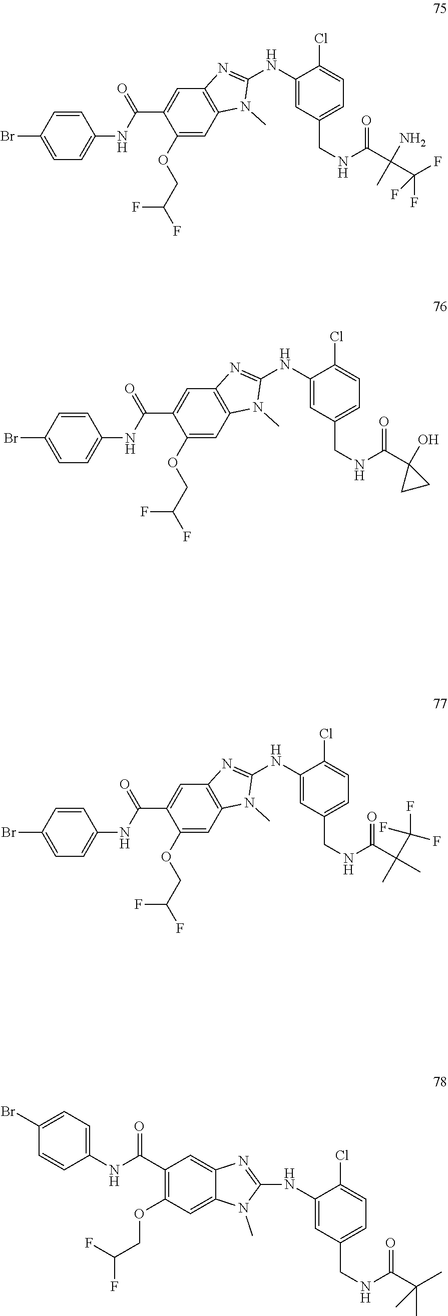

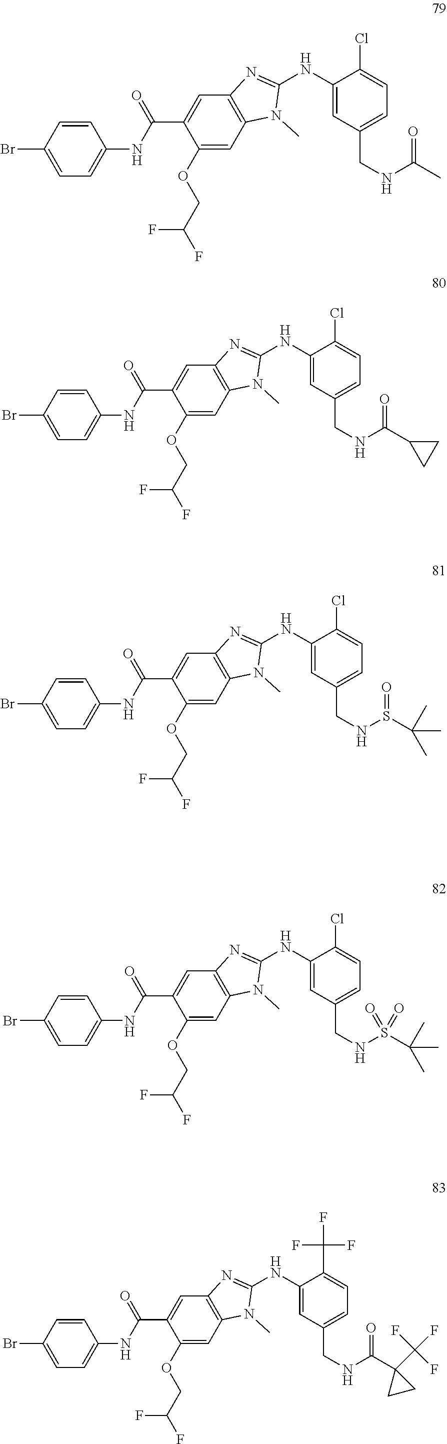 [SMILES: CC(=O)NCc1ccc(Cl)c(Nc2nc3cc(C(=O)Nc4ccc(Br)cc4)c(OCC(F)F)cc3n2C)c1 ; Cn1c(Nc2cc(CNC(=O)C3CC3)ccc2Cl)nc2cc(C(=O)Nc3ccc(Br)cc3)c(OCC(F)F)cc21 ; Cn1c(Nc2cc(CNS(=O)C(C)(C)C)ccc2Cl)nc2cc(C(=O)Nc3ccc(Br)cc3)c(OCC(F)F)cc21 ; Cn1c(Nc2cc(CNS(=O)(=O)C(C)(C)C)ccc2Cl)nc2cc(C(=O)Nc3ccc(Br)cc3)c(OCC(F)F)cc21 ; Cn1c(Nc2cc(CNC(=O)C3(C(F)(F)F)CC3)ccc2C(F)(F)F)nc2cc(C(=O)Nc3ccc(Br)cc3)c(OCC(F)F)cc21]

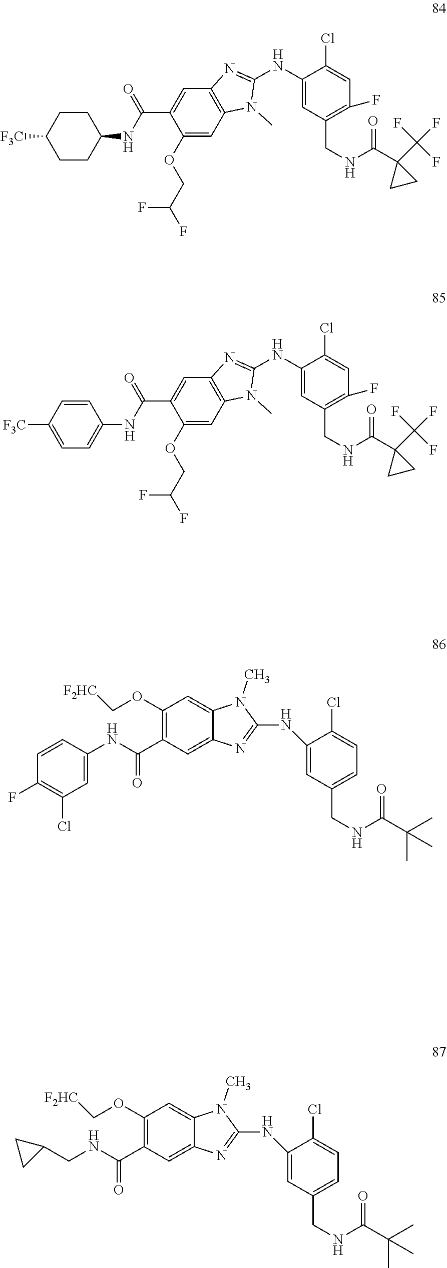 [SMILES: Cn1c(Nc2cc(CNC(=O)C3(C(F)(F)F)CC3)c(F)cc2Cl)nc2cc(C(=O)N[C@H]3CC[C@H](C(F)(F)F)CC3)c(OCC(F)F)cc21 ; Cn1c(Nc2cc(CNC(=O)C3(C(F)(F)F)CC3)c(F)cc2Cl)nc2cc(C(=O)Nc3ccc(C(F)(F)F)cc3)c(OCC(F)F)cc21 ; Cn1c(Nc2cc(CNC(=O)C(C)(C)C)ccc2Cl)nc2cc(C(=O)Nc3ccc(F)c(Cl)c3)c(OCC(F)F)cc21 ; Cn1c(Nc2cc(CNC(=O)C(C)(C)C)ccc2Cl)nc2cc(C(=O)NCC3CC3)c(OCC(F)F)cc21]

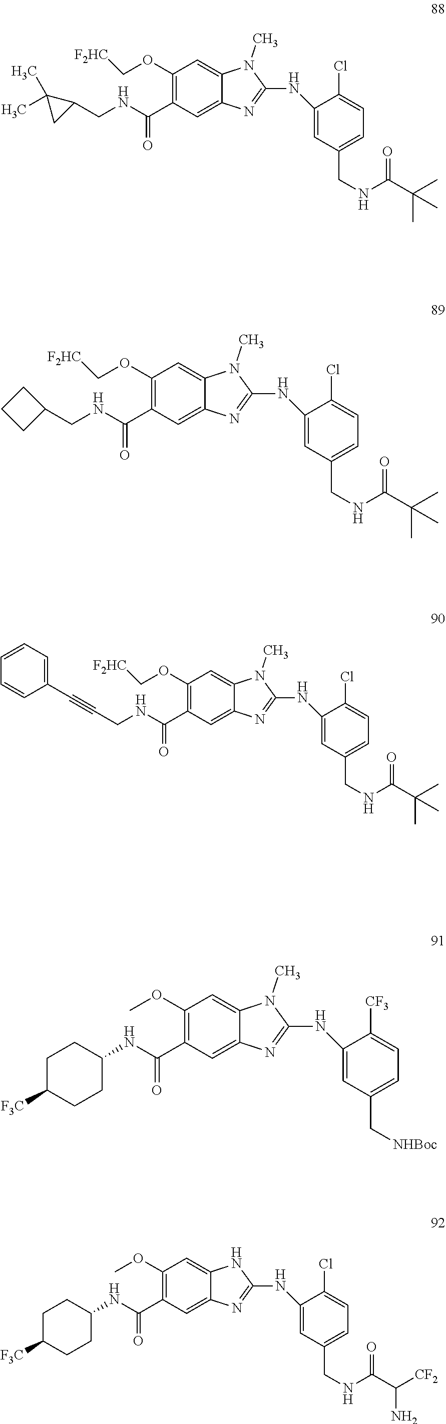

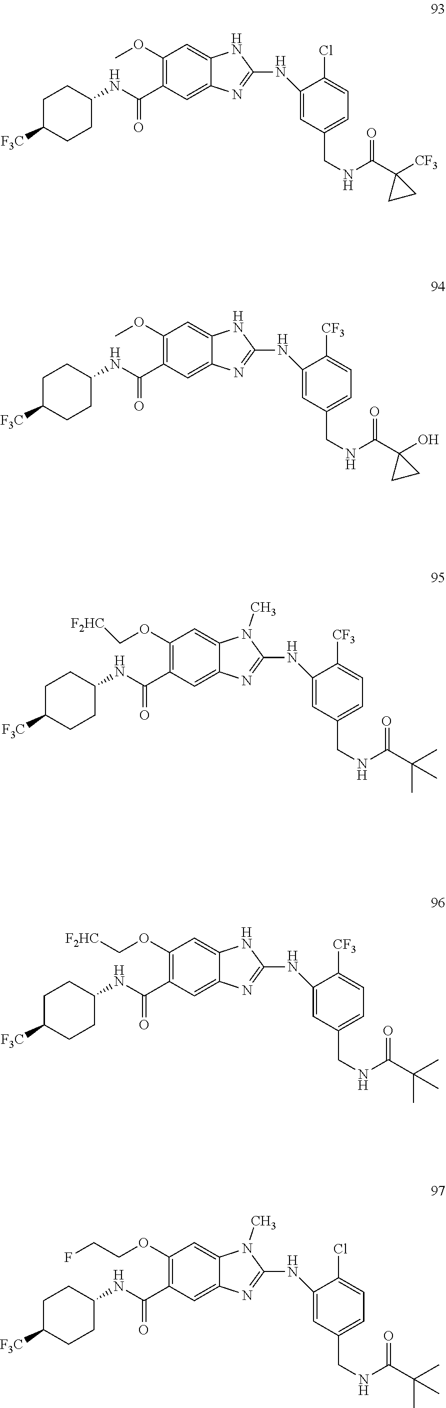 [SMILES: COc1cc2[nH]c(Nc3cc(CNC(=O)C4(C(F)(F)F)CC4)ccc3Cl)nc2cc1C(=O)N[C@H]1CC[C@H](C(F)(F)F)CC1 ; COc1cc2[nH]c(Nc3cc(CNC(=O)C4(O)CC4)ccc3C(F)(F)F)nc2cc1C(=O)N[C@H]1CC[C@H](C(F)(F)F)CC1 ; Cn1c(Nc2cc(CNC(=O)C(C)(C)C)ccc2C(F)(F)F)nc2cc(C(=O)N[C@H]3CC[C@H](C(F)(F)F)CC3)c(OCC(F)F)cc21 ; CC(C)(C)C(=O)NCc1ccc(C(F)(F)F)c(Nc2nc3cc(C(=O)N[C@H]4CC[C@H](C(F)(F)F)CC4)c(OCC(F)F)cc3[nH]2)c1 ; Cn1c(Nc2cc(CNC(=O)C(C)(C)C)ccc2Cl)nc2cc(C(=O)N[C@H]3CC[C@H](C(F)(F)F)CC3)c(OCCF)cc21]

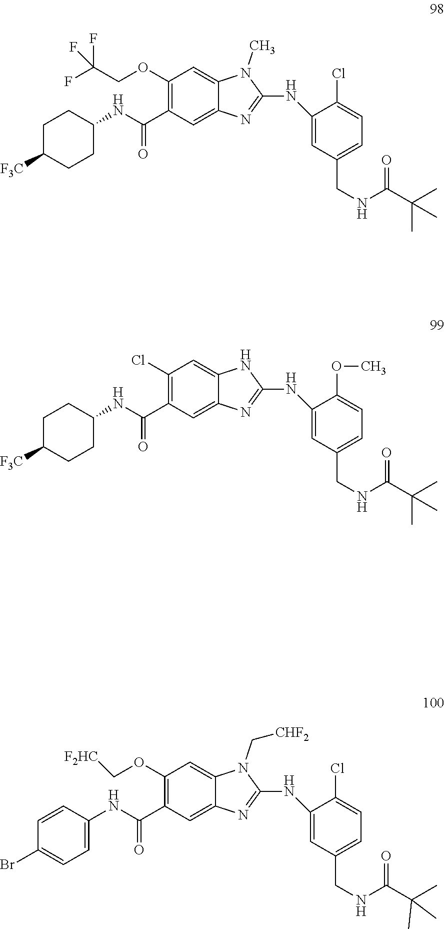 [SMILES: Cn1c(Nc2cc(CNC(=O)C(C)(C)C)ccc2Cl)nc2cc(C(=O)N[C@H]3CC[C@H](C(F)(F)F)CC3)c(OCC(F)(F)F)cc21 ; COc1ccc(CNC(=O)C(C)(C)C)cc1Nc1nc2cc(C(=O)N[C@H]3CC[C@H](C(F)(F)F)CC3)c(Cl)cc2[nH]1 ; CC(C)(C)C(=O)NCc1ccc(Cl)c(Nc2nc3cc(C(=O)Nc4ccc(Br)cc4)c(OCC(F)F)cc3n2CC(F)F)c1]

In further embodiments, compounds that may be utilised as the required mPGES-1 inhibitor may include the following compounds as numbered in the tables together forming Group B below, or a pharmaceutically acceptable salt thereof:

| | Structure |
|---|---|
| 1 | 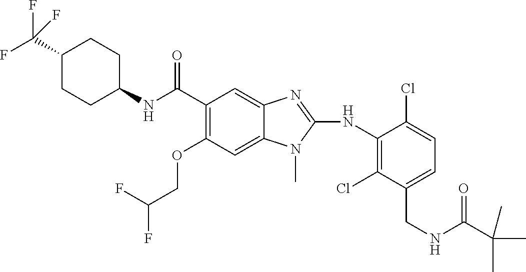 [SMILES: Cn1c(Nc2c(Cl)ccc(CNC(=O)C(C)(C)C)c2Cl)nc2cc(C(=O)N[C@H]3CC[C@H](C(F)(F)F)CC3)c(OCC(F)F)cc21] |
| 2 | 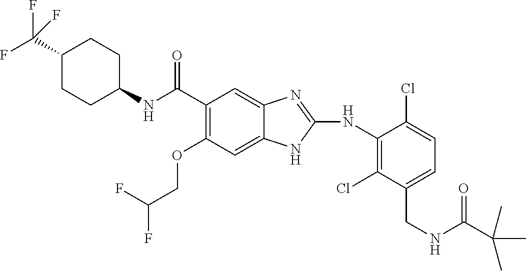 [SMILES: CC(C)(C)C(=O)NCc1ccc(Cl)c(Nc2nc3cc(C(=O)N[C@H]4CC[C@H](C(F)(F)F)CC4)c(OCC(F)F)cc3[nH]2)c1Cl] |
| 3 | 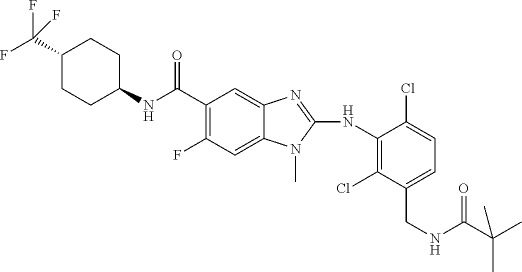 [SMILES: Cn1c(Nc2c(Cl)ccc(CNC(=O)C(C)(C)C)c2Cl)nc2cc(C(=O)N[C@H]3CC[C@H](C(F)(F)F)CC3)c(F)cc21] |
| 4 | 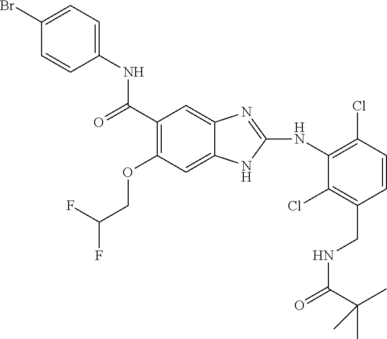 [SMILES: CC(C)(C)C(=O)NCc1ccc(Cl)c(Nc2nc3cc(C(=O)Nc4ccc(Br)cc4)c(OCC(F)F)cc3[nH]2)c1Cl] |
| 5 | 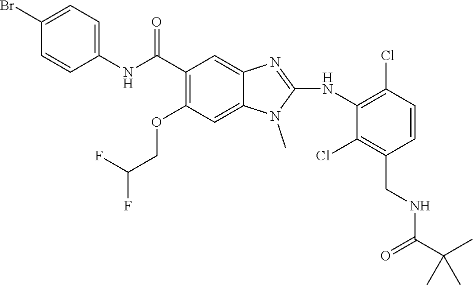 [SMILES: Cn1c(Nc2c(Cl)ccc(CNC(=O)C(C)(C)C)c2Cl)nc2cc(C(=O)Nc3ccc(Br)cc3)c(OCC(F)F)cc21] |
| 6 | 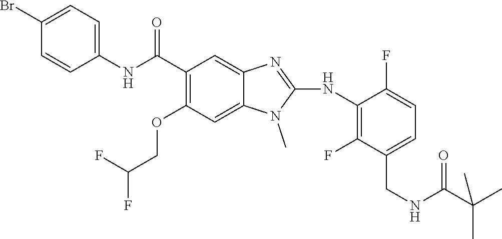 [SMILES: Cn1c(Nc2c(F)ccc(CNC(=O)C(C)(C)C)c2F)nc2cc(C(=O)Nc3ccc(Br)cc3)c(OCC(F)F)cc21] |
| 7 | 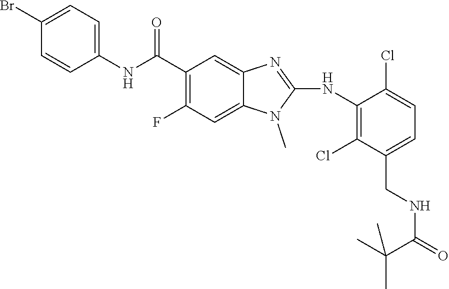 [SMILES: Cn1c(Nc2c(Cl)ccc(CNC(=O)C(C)(C)C)c2Cl)nc2cc(C(=O)Nc3ccc(Br)cc3)c(F)cc21] |
| 8 | 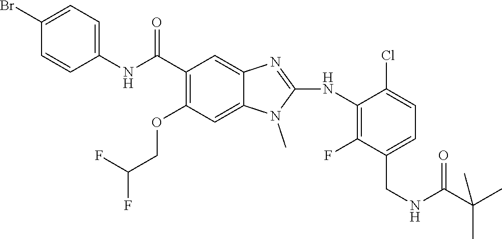 [SMILES: Cn1c(Nc2c(Cl)ccc(CNC(=O)C(C)(C)C)c2F)nc2cc(C(=O)Nc3ccc(Br)cc3)c(OCC(F)F)cc21] |
| 9 | 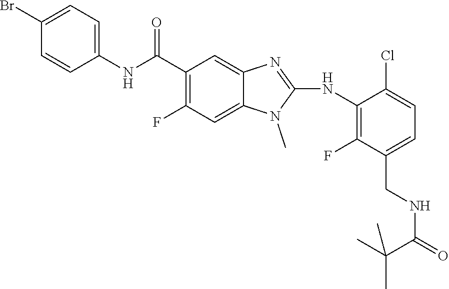 [SMILES: Cn1c(Nc2c(Cl)ccc(CNC(=O)C(C)(C)C)c2F)nc2cc(C(=O)Nc3ccc(Br)cc3)c(F)cc21] |
| 10 | 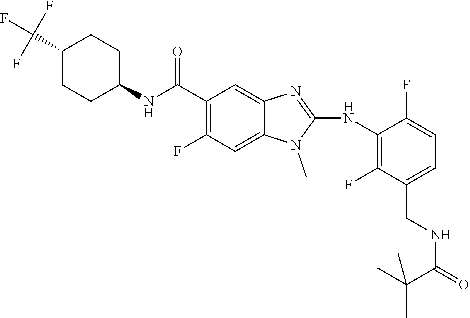 [SMILES: Cn1c(Nc2c(F)ccc(CNC(=O)C(C)(C)C)c2F)nc2cc(C(=O)N[C@H]3CC[C@H](C(F)(F)F)CC3)c(F)cc21] |
| 11 | 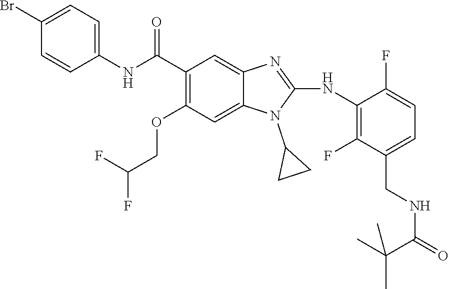 [SMILES: CC(C)(C)C(=O)NCc1ccc(F)c(Nc2nc3cc(C(=O)Nc4ccc(Br)cc4)c(OCC(F)F)cc3n2C2CC2)c1F] |
| 12 | 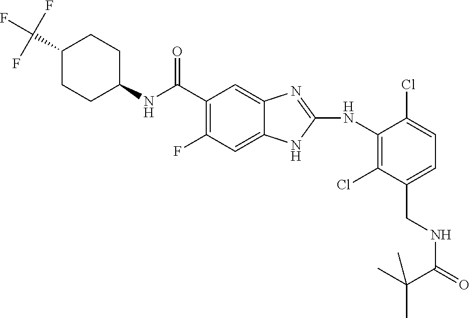 [SMILES: CC(C)(C)C(=O)NCc1ccc(Cl)c(Nc2nc3cc(C(=O)N[C@H]4CC[C@H](C(F)(F)F)CC4)c(F)cc3[nH]2)c1Cl] |
| 13 | 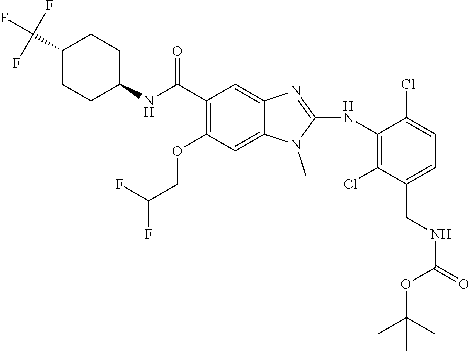 [SMILES: Cn1c(Nc2c(Cl)ccc(CNC(=O)OC(C)(C)C)c2Cl)nc2cc(C(=O)N[C@H]3CC[C@H](C(F)(F)F)CC3)c(OCC(F)F)cc21] |
| 14 | 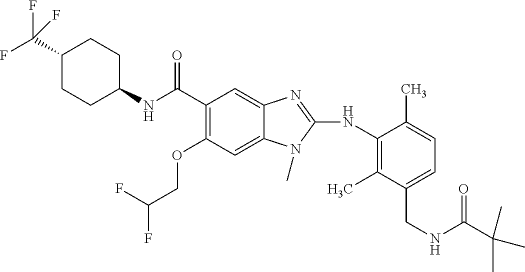 [SMILES: Cc1ccc(CNC(=O)C(C)(C)C)c(C)c1Nc1nc2cc(C(=O)N[C@H]3CC[C@H](C(F)(F)F)CC3)c(OCC(F)F)cc2n1C] |
| 15 | 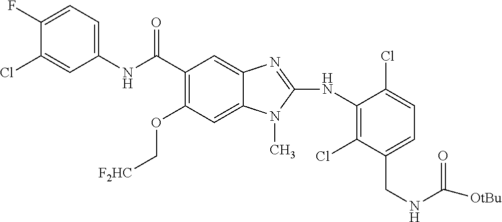 [SMILES: Cn1c(Nc2c(Cl)ccc(CNC(=O)OC(C)(C)C)c2Cl)nc2cc(C(=O)Nc3ccc(F)c(Cl)c3)c(OCC(F)F)cc21] |
| 16 | 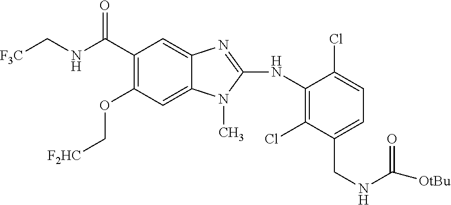 [SMILES: Cn1c(Nc2c(Cl)ccc(CNC(=O)OC(C)(C)C)c2Cl)nc2cc(C(=O)NCC(F)(F)F)c(OCC(F)F)cc21] |
| 17 | 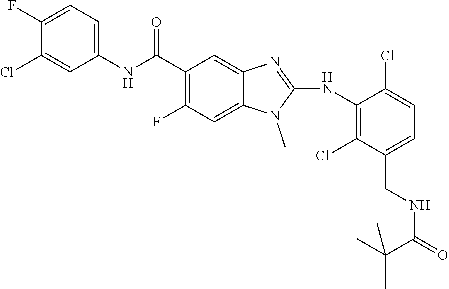 [SMILES: Cn1c(Nc2c(Cl)ccc(CNC(=O)C(C)(C)C)c2Cl)nc2cc(C(=O)Nc3ccc(F)c(Cl)c3)c(F)cc21] |
| 18 | 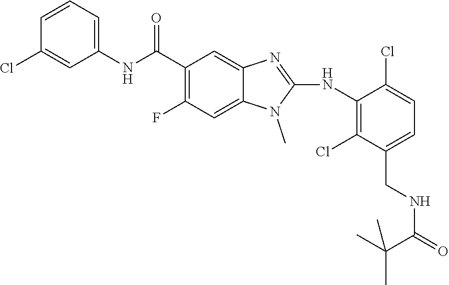 [SMILES: Cn1c(Nc2c(Cl)ccc(CNC(=O)C(C)(C)C)c2Cl)nc2cc(C(=O)Nc3cccc(Cl)c3)c(F)cc21] |
| 19 | 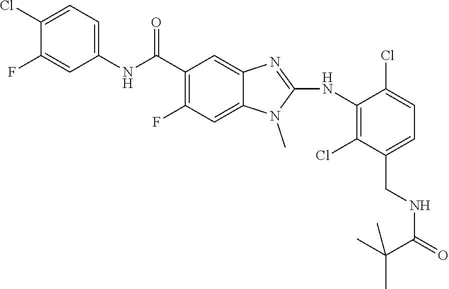 [SMILES: Cn1c(Nc2c(Cl)ccc(CNC(=O)C(C)(C)C)c2Cl)nc2cc(C(=O)Nc3ccc(Cl)c(F)c3)c(F)cc21] |
| 20 | 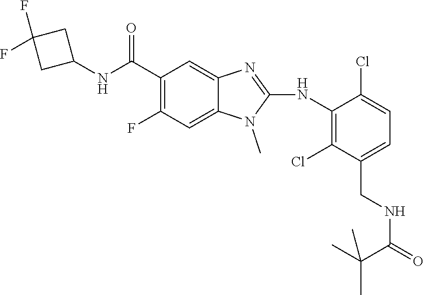 [SMILES: Cn1c(Nc2c(Cl)ccc(CNC(=O)C(C)(C)C)c2Cl)nc2cc(C(=O)NC3CC(F)(F)C3)c(F)cc21] |
| 21 | 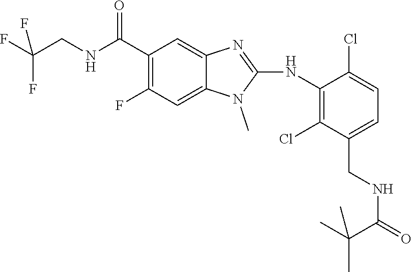 [SMILES: Cn1c(Nc2c(Cl)ccc(CNC(=O)C(C)(C)C)c2Cl)nc2cc(C(=O)NCC(F)(F)F)c(F)cc21] |
| 22 | 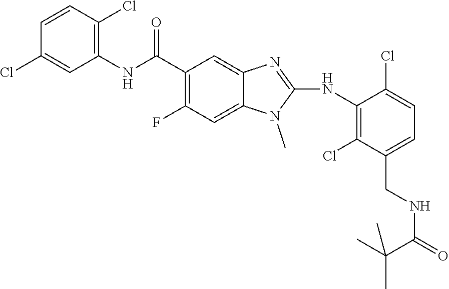 [SMILES: Cn1c(Nc2c(Cl)ccc(CNC(=O)C(C)(C)C)c2Cl)nc2cc(C(=O)Nc3cc(Cl)ccc3Cl)c(F)cc21] |
| 23 | 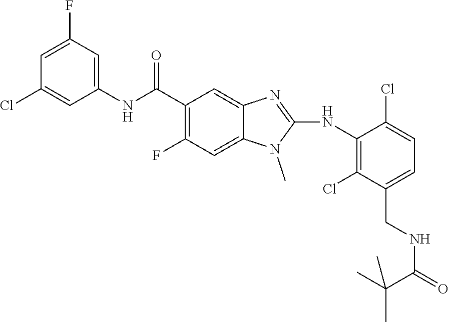 [SMILES: Cn1c(Nc2c(Cl)ccc(CNC(=O)C(C)(C)C)c2Cl)nc2cc(C(=O)Nc3cc(F)cc(Cl)c3)c(F)cc21] |
| 24 | 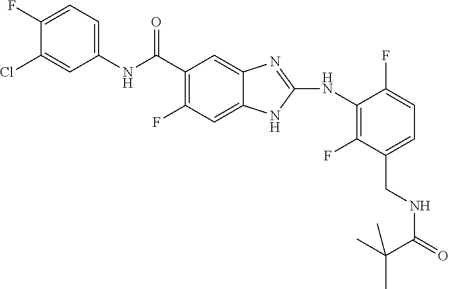 [SMILES: CC(C)(C)C(=O)NCc1ccc(F)c(Nc2nc3cc(C(=O)Nc4ccc(F)c(Cl)c4)c(F)cc3[nH]2)c1F] |
| 25 | 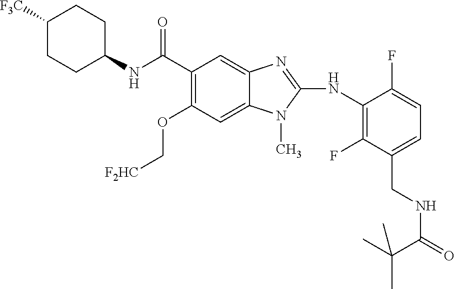 [SMILES: Cn1c(Nc2c(F)ccc(CNC(=O)C(C)(C)C)c2F)nc2cc(C(=O)N[C@H]3CC[C@H](C(F)(F)F)CC3)c(OCC(F)F)cc21] |
| 26 | 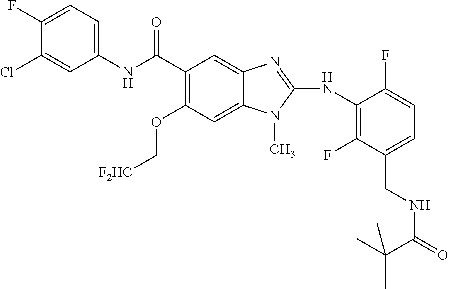 [SMILES: Cn1c(Nc2c(F)ccc(CNC(=O)C(C)(C)C)c2F)nc2cc(C(=O)Nc3ccc(F)c(Cl)c3)c(OCC(F)F)cc21] |
| 27 | 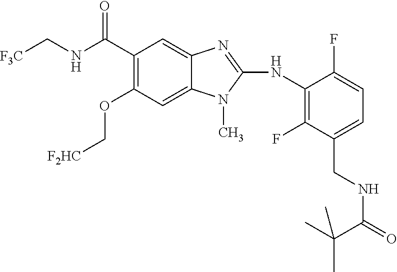 [SMILES: Cn1c(Nc2c(F)ccc(CNC(=O)C(C)(C)C)c2F)nc2cc(C(=O)NCC(F)(F)F)c(OCC(F)F)cc21] |
| 28 | 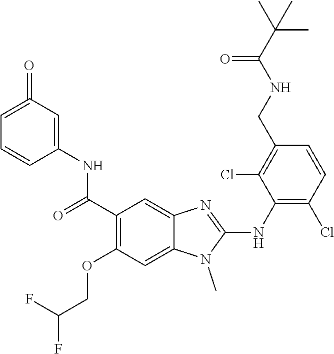 |
| 29 | 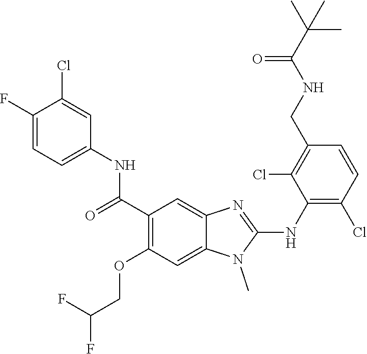 [SMILES: Cn1c(Nc2c(Cl)ccc(CNC(=O)C(C)(C)C)c2Cl)nc2cc(C(=O)Nc3ccc(F)c(Cl)c3)c(OCC(F)F)cc21] |
| 30 | 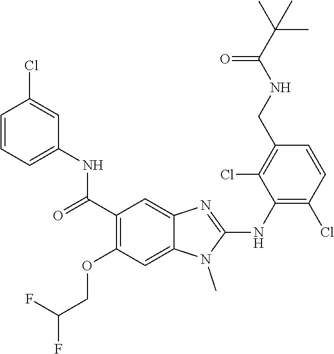 [SMILES: Cn1c(Nc2c(Cl)ccc(CNC(=O)C(C)(C)C)c2Cl)nc2cc(C(=O)Nc3cccc(Cl)c3)c(OCC(F)F)cc21] |
| 31 | 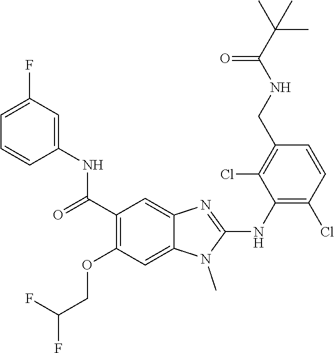 [SMILES: Cn1c(Nc2c(Cl)ccc(CNC(=O)C(C)(C)C)c2Cl)nc2cc(C(=O)Nc3cccc(F)c3)c(OCC(F)F)cc21] |
| 32 | 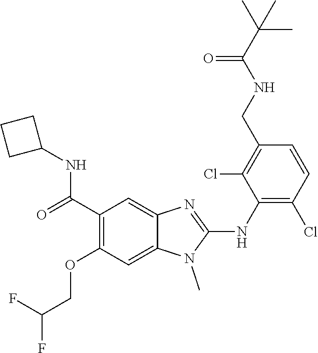 [SMILES: Cn1c(Nc2c(Cl)ccc(CNC(=O)C(C)(C)C)c2Cl)nc2cc(C(=O)NC3CCC3)c(OCC(F)F)cc21] |
| 33 | 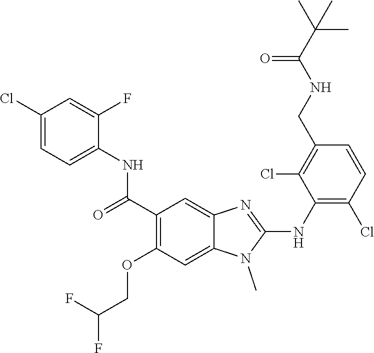 [SMILES: Cn1c(Nc2c(Cl)ccc(CNC(=O)C(C)(C)C)c2Cl)nc2cc(C(=O)Nc3ccc(Cl)cc3F)c(OCC(F)F)cc21] |
| 34 | 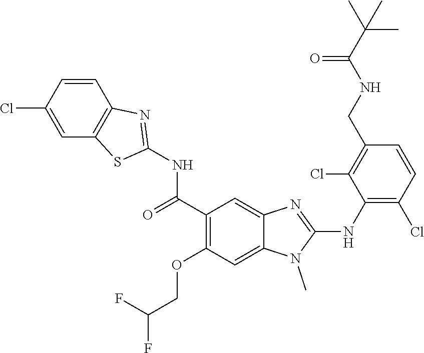 [SMILES: Cn1c(Nc2c(Cl)ccc(CNC(=O)C(C)(C)C)c2Cl)nc2cc(C(=O)Nc3nc4ccc(Cl)cc4s3)c(OCC(F)F)cc21] |
| 35 | 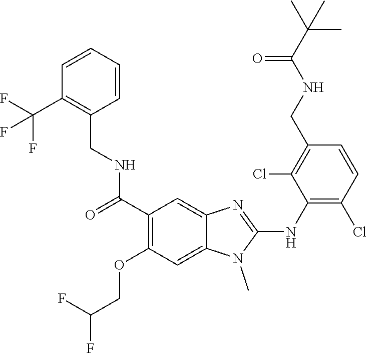 [SMILES: Cn1c(Nc2c(Cl)ccc(CNC(=O)C(C)(C)C)c2Cl)nc2cc(C(=O)NCc3ccccc3C(F)(F)F)c(OCC(F)F)cc21] |
| 36 | 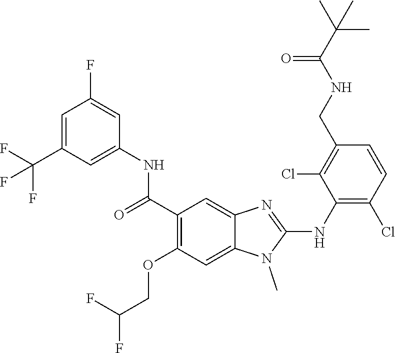 [SMILES: Cn1c(Nc2c(Cl)ccc(CNC(=O)C(C)(C)C)c2Cl)nc2cc(C(=O)Nc3cc(F)cc(C(F)(F)F)c3)c(OCC(F)F)cc21] |
| 37 | 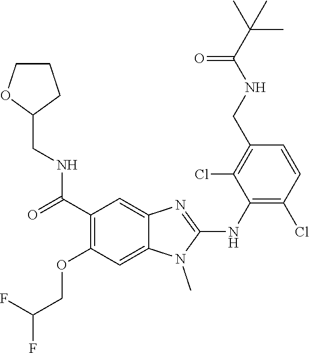 [SMILES: Cn1c(Nc2c(Cl)ccc(CNC(=O)C(C)(C)C)c2Cl)nc2cc(C(=O)NCC3CCCO3)c(OCC(F)F)cc21] |
| 38 | 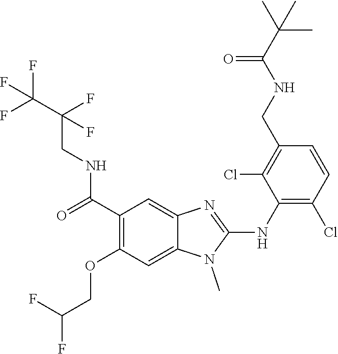 [SMILES: Cn1c(Nc2c(Cl)ccc(CNC(=O)C(C)(C)C)c2Cl)nc2cc(C(=O)NCC(F)(F)C(F)(F)F)c(OCC(F)F)cc21] |
| 39 | 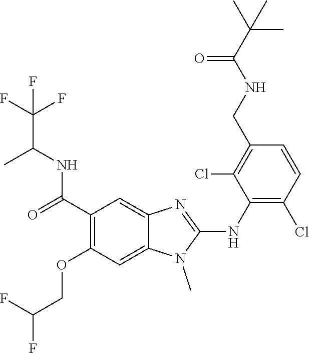 [SMILES: CC(NC(=O)c1cc2nc(Nc3c(Cl)ccc(CNC(=O)C(C)(C)C)c3Cl)n(C)c2cc1OCC(F)F)C(F)(F)F] |
| 40 | 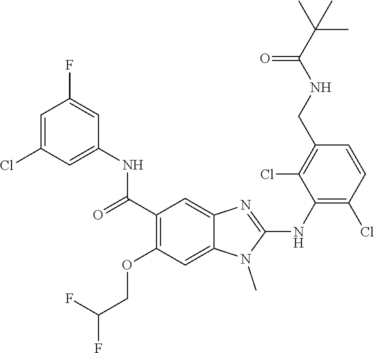 [SMILES: Cn1c(Nc2c(Cl)ccc(CNC(=O)C(C)(C)C)c2Cl)nc2cc(C(=O)Nc3cc(F)cc(Cl)c3)c(OCC(F)F)cc21] |
| 41 | 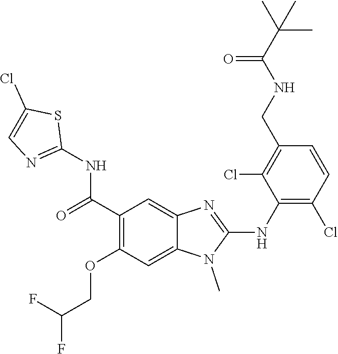 [SMILES: Cn1c(Nc2c(Cl)ccc(CNC(=O)C(C)(C)C)c2Cl)nc2cc(C(=O)Nc3ncc(Cl)s3)c(OCC(F)F)cc21] |
| 42 | 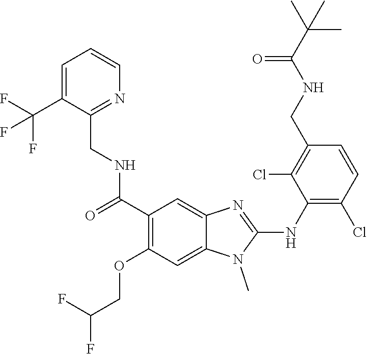 [SMILES: Cn1c(Nc2c(Cl)ccc(CNC(=O)C(C)(C)C)c2Cl)nc2cc(C(=O)NCc3ncccc3C(F)(F)F)c(OCC(F)F)cc21] |
| 43 | 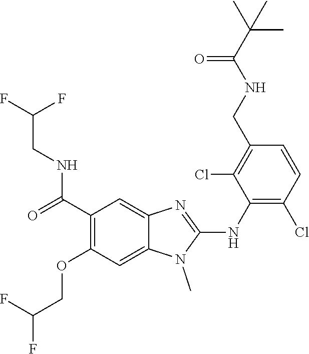 [SMILES: Cn1c(Nc2c(Cl)ccc(CNC(=O)C(C)(C)C)c2Cl)nc2cc(C(=O)NCC(F)F)c(OCC(F)F)cc21] |
| 44 | 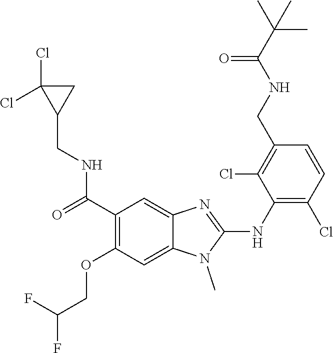 [SMILES: Cn1c(Nc2c(Cl)ccc(CNC(=O)C(C)(C)C)c2Cl)nc2cc(C(=O)NCC3CC3(Cl)Cl)c(OCC(F)F)cc21] |
| 45 | 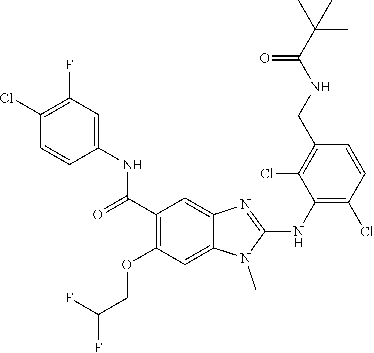 [SMILES: Cn1c(Nc2c(Cl)ccc(CNC(=O)C(C)(C)C)c2Cl)nc2cc(C(=O)Nc3ccc(Cl)c(F)c3)c(OCC(F)F)cc21] |
| 46 | 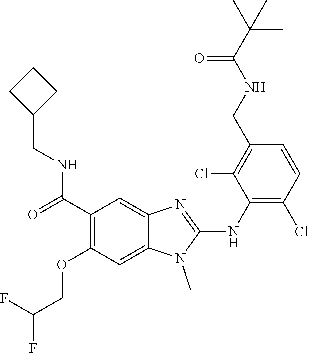 [SMILES: Cn1c(Nc2c(Cl)ccc(CNC(=O)C(C)(C)C)c2Cl)nc2cc(C(=O)NCC3CCC3)c(OCC(F)F)cc21] |
| 47 | 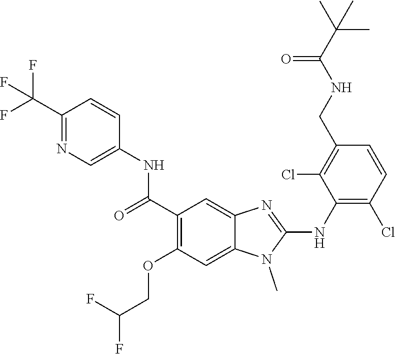 [SMILES: Cn1c(Nc2c(Cl)ccc(CNC(=O)C(C)(C)C)c2Cl)nc2cc(C(=O)Nc3ccc(C(F)(F)F)nc3)c(OCC(F)F)cc21] |
| 48 | 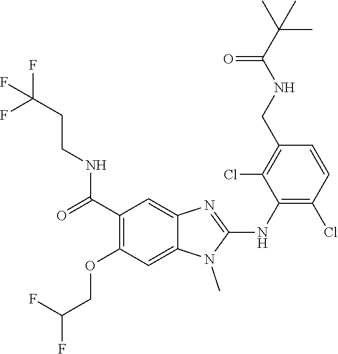 [SMILES: Cn1c(Nc2c(Cl)ccc(CNC(=O)C(C)(C)C)c2Cl)nc2cc(C(=O)NCCC(F)(F)F)c(OCC(F)F)cc21] |
| 49 | 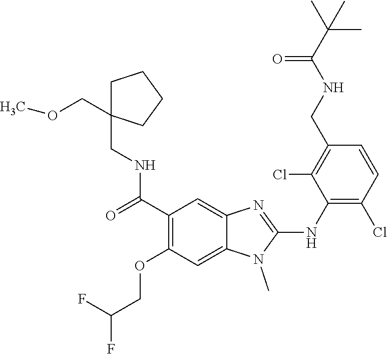 [SMILES: COCC1(CNC(=O)c2cc3nc(Nc4c(Cl)ccc(CNC(=O)C(C)(C)C)c4Cl)n(C)c3cc2OCC(F)F)CCCC1] |
| 50 | 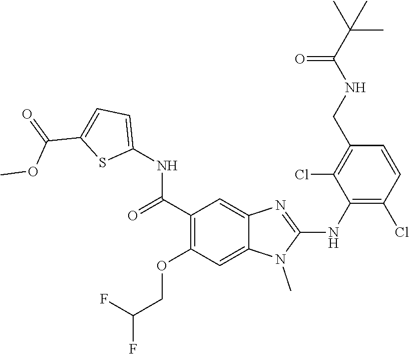 [SMILES: COC(=O)c1ccc(NC(=O)c2cc3nc(Nc4c(Cl)ccc(CNC(=O)C(C)(C)C)c4Cl)n(C)c3cc2OCC(F)F)s1] |
| 51 | 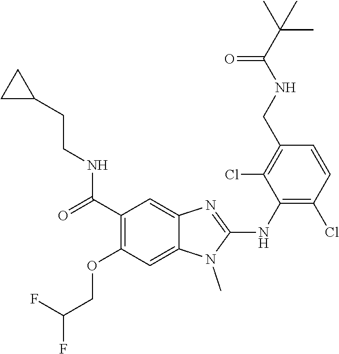 [SMILES: Cn1c(Nc2c(Cl)ccc(CNC(=O)C(C)(C)C)c2Cl)nc2cc(C(=O)NCCC3CC3)c(OCC(F)F)cc21] |
| 52 | 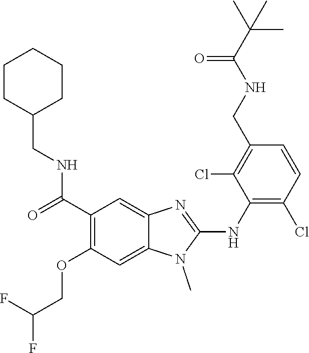 [SMILES: Cn1c(Nc2c(Cl)ccc(CNC(=O)C(C)(C)C)c2Cl)nc2cc(C(=O)NCC3CCCCC3)c(OCC(F)F)cc21] |
| 53 | 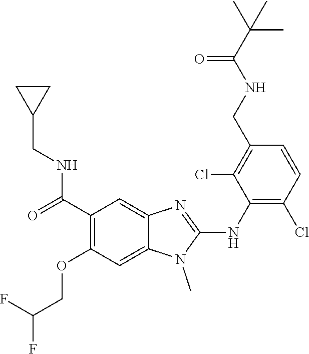 [SMILES: Cn1c(Nc2c(Cl)ccc(CNC(=O)C(C)(C)C)c2Cl)nc2cc(C(=O)NCC3CC3)c(OCC(F)F)cc21] |
| 54 | 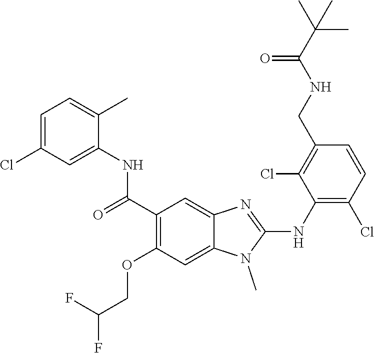 [SMILES: Cc1ccc(Cl)cc1NC(=O)c1cc2nc(Nc3c(Cl)ccc(CNC(=O)C(C)(C)C)c3Cl)n(C)c2cc1OCC(F)F] |
| 55 | 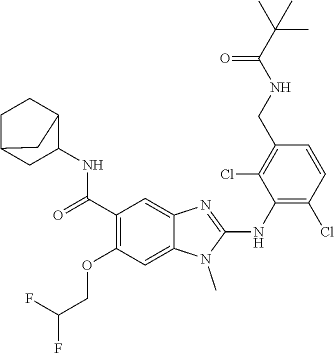 [SMILES: Cn1c(Nc2c(Cl)ccc(CNC(=O)C(C)(C)C)c2Cl)nc2cc(C(=O)NC3CC4CCC3C4)c(OCC(F)F)cc21] |
| 56 | 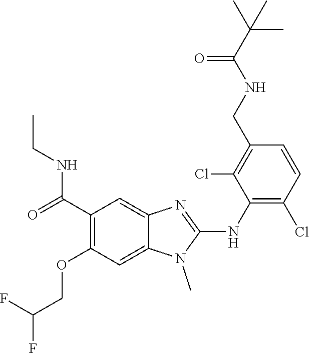 [SMILES: CCNC(=O)c1cc2nc(Nc3c(Cl)ccc(CNC(=O)C(C)(C)C)c3Cl)n(C)c2cc1OCC(F)F] |
| 57 | 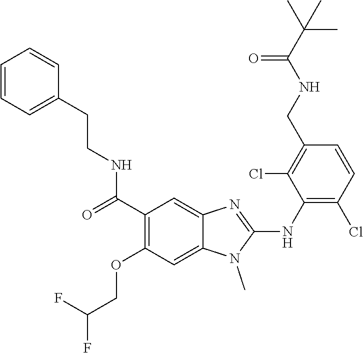 [SMILES: Cn1c(Nc2c(Cl)ccc(CNC(=O)C(C)(C)C)c2Cl)nc2cc(C(=O)NCCc3ccccc3)c(OCC(F)F)cc21] |
| 58 | 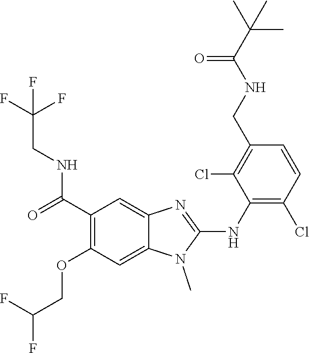 [SMILES: Cn1c(Nc2c(Cl)ccc(CNC(=O)C(C)(C)C)c2Cl)nc2cc(C(=O)NCC(F)(F)F)c(OCC(F)F)cc21] |
| 59 | 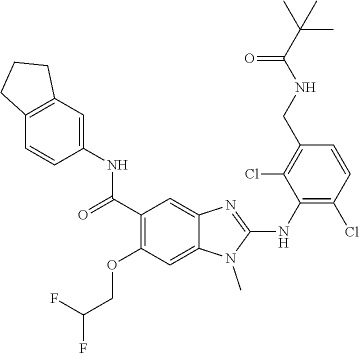 [SMILES: Cn1c(Nc2c(Cl)ccc(CNC(=O)C(C)(C)C)c2Cl)nc2cc(C(=O)Nc3ccc4c(c3)CCC4)c(OCC(F)F)cc21] |
| 60 | 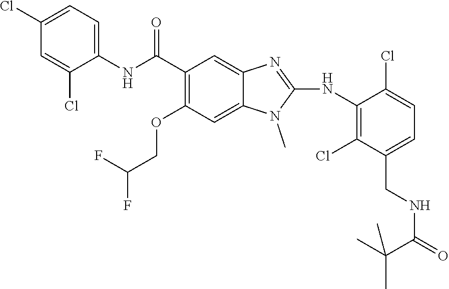 [SMILES: Cn1c(Nc2c(Cl)ccc(CNC(=O)C(C)(C)C)c2Cl)nc2cc(C(=O)Nc3ccc(Cl)cc3Cl)c(OCC(F)F)cc21] |
| 61 | 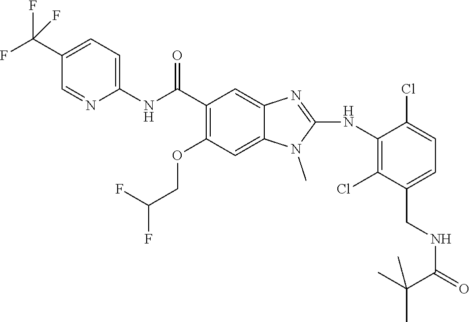 [SMILES: Cn1c(Nc2c(Cl)ccc(CNC(=O)C(C)(C)C)c2Cl)nc2cc(C(=O)Nc3ccc(C(F)(F)F)cn3)c(OCC(F)F)cc21] |
| 62 | 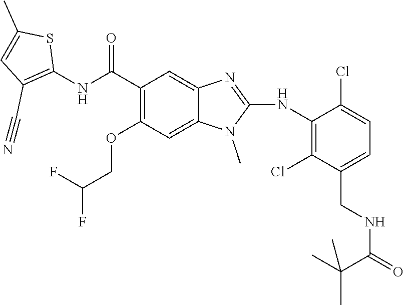 [SMILES: Cc1cc(C#N)c(NC(=O)c2cc3nc(Nc4c(Cl)ccc(CNC(=O)C(C)(C)C)c4Cl)n(C)c3cc2OCC(F)F)s1] |
| 63 | 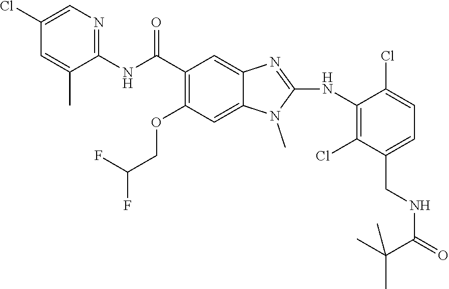 [SMILES: Cc1cc(Cl)cnc1NC(=O)c1cc2nc(Nc3c(Cl)ccc(CNC(=O)C(C)(C)C)c3Cl)n(C)c2cc1OCC(F)F] |
| 64 | 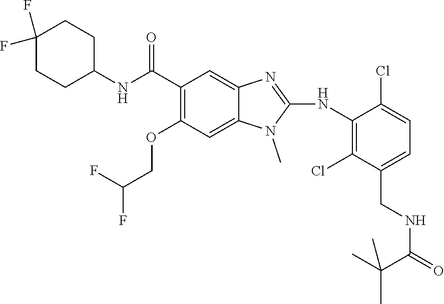 [SMILES: Cn1c(Nc2c(Cl)ccc(CNC(=O)C(C)(C)C)c2Cl)nc2cc(C(=O)NC3CCC(F)(F)CC3)c(OCC(F)F)cc21] |
| 65 | 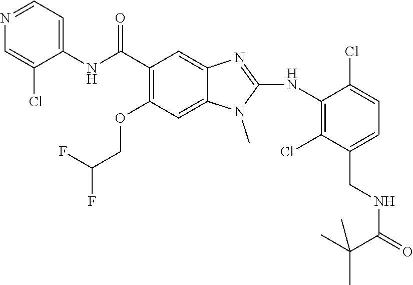 [SMILES: Cn1c(Nc2c(Cl)ccc(CNC(=O)C(C)(C)C)c2Cl)nc2cc(C(=O)Nc3ccncc3Cl)c(OCC(F)F)cc21] |
| 66 | 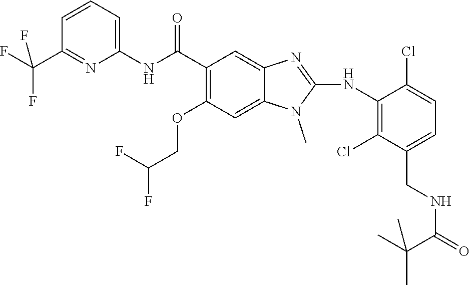 [SMILES: Cn1c(Nc2c(Cl)ccc(CNC(=O)C(C)(C)C)c2Cl)nc2cc(C(=O)Nc3cccc(C(F)(F)F)n3)c(OCC(F)F)cc21] |
| 67 | 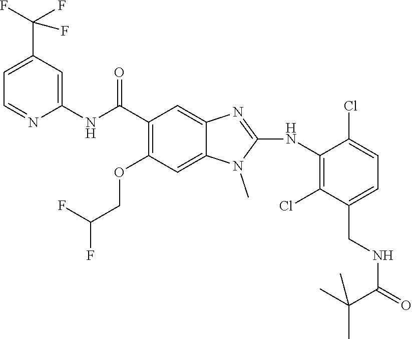 [SMILES: Cn1c(Nc2c(Cl)ccc(CNC(=O)C(C)(C)C)c2Cl)nc2cc(C(=O)Nc3cc(C(F)(F)F)ccn3)c(OCC(F)F)cc21] |
| 68 | 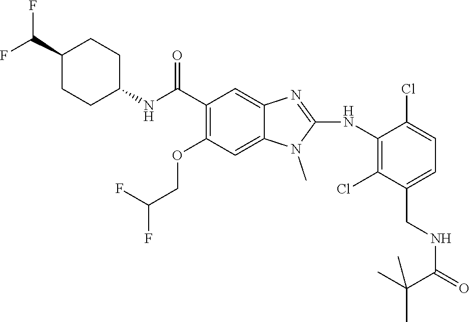 [SMILES: Cn1c(Nc2c(Cl)ccc(CNC(=O)C(C)(C)C)c2Cl)nc2cc(C(=O)N[C@H]3CC[C@H](C(F)F)CC3)c(OCC(F)F)cc21] |
| 69 | 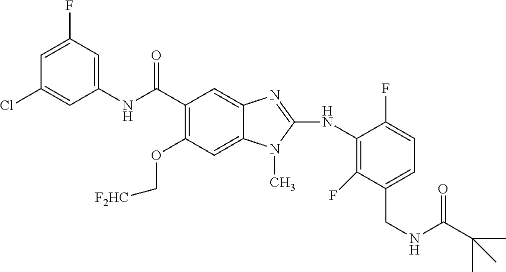 [SMILES: Cn1c(Nc2c(F)ccc(CNC(=O)C(C)(C)C)c2F)nc2cc(C(=O)Nc3cc(F)cc(Cl)c3)c(OCC(F)F)cc21] |
| 70 | 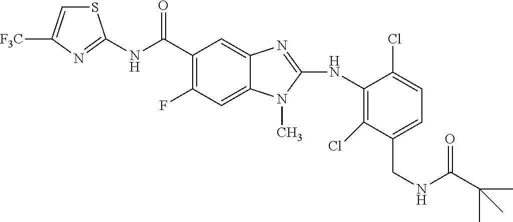 [SMILES: Cn1c(Nc2c(Cl)ccc(CNC(=O)C(C)(C)C)c2Cl)nc2cc(C(=O)Nc3nc(C(F)(F)F)cs3)c(F)cc21] |
| 71 | 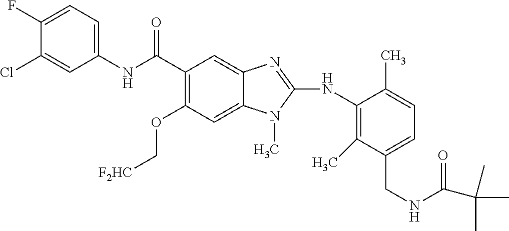 [SMILES: Cc1ccc(CNC(=O)C(C)(C)C)c(C)c1Nc1nc2cc(C(=O)Nc3ccc(F)c(Cl)c3)c(OCC(F)F)cc2n1C] |
| 72 | 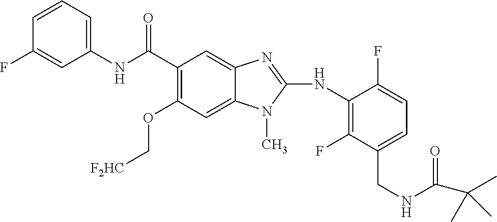 [SMILES: Cn1c(Nc2c(F)ccc(CNC(=O)C(C)(C)C)c2F)nc2cc(C(=O)Nc3cccc(F)c3)c(OCC(F)F)cc21] |
| 73 | 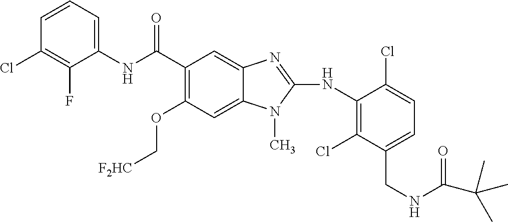 [SMILES: Cn1c(Nc2c(Cl)ccc(CNC(=O)C(C)(C)C)c2Cl)nc2cc(C(=O)Nc3cccc(Cl)c3F)c(OCC(F)F)cc21] |
| 74 | 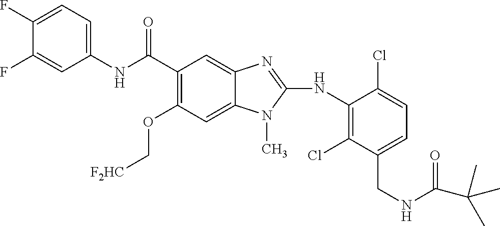 [SMILES: Cn1c(Nc2c(Cl)ccc(CNC(=O)C(C)(C)C)c2Cl)nc2cc(C(=O)Nc3ccc(F)c(F)c3)c(OCC(F)F)cc21] |
| 75 | 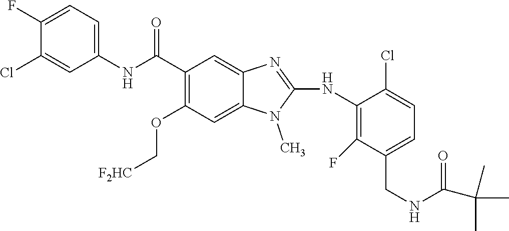 [SMILES: Cn1c(Nc2c(Cl)ccc(CNC(=O)C(C)(C)C)c2F)nc2cc(C(=O)Nc3ccc(F)c(Cl)c3)c(OCC(F)F)cc21] |
| 76 | 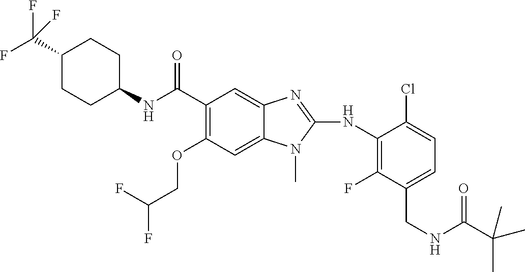 [SMILES: Cn1c(Nc2c(Cl)ccc(CNC(=O)C(C)(C)C)c2F)nc2cc(C(=O)N[C@H]3CC[C@H](C(F)(F)F)CC3)c(OCC(F)F)cc21] |
| 77 | 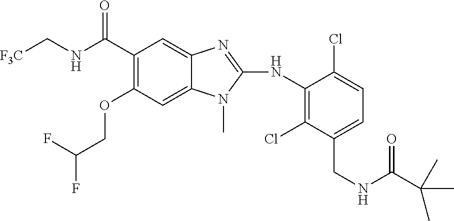 [SMILES: Cn1c(Nc2c(Cl)ccc(CNC(=O)C(C)(C)C)c2Cl)nc2cc(C(=O)NCC(F)(F)F)c(OCC(F)F)cc21] |
| 78 | 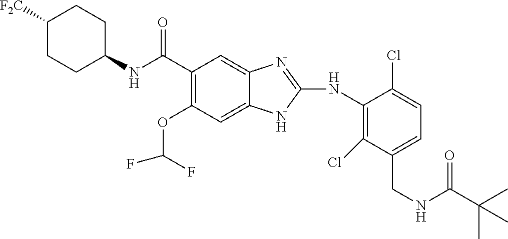 |
| 79 | 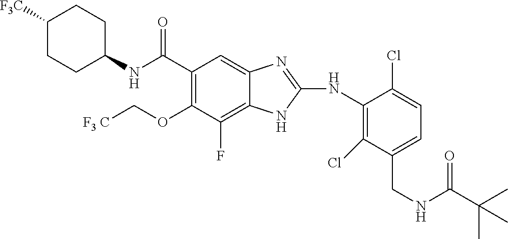 |
| 80 | 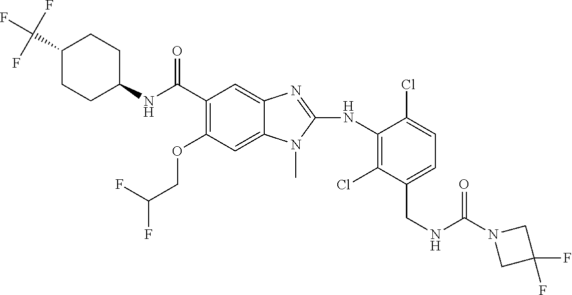 [SMILES: Cn1c(Nc2c(Cl)ccc(CNC(=O)N3CC(F)(F)C3)c2Cl)nc2cc(C(=O)N[C@H]3CC[C@H](C(F)(F)F)CC3)c(OCC(F)F)cc21] |
| 81 | 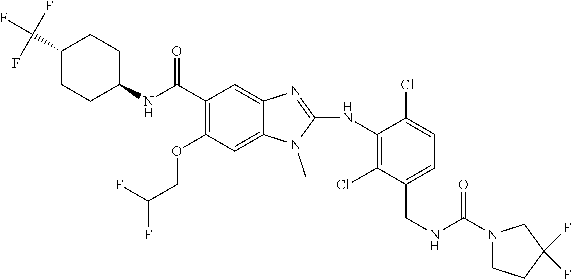 [SMILES: Cn1c(Nc2c(Cl)ccc(CNC(=O)N3CCC(F)(F)C3)c2Cl)nc2cc(C(=O)N[C@H]3CC[C@H](C(F)(F)F)CC3)c(OCC(F)F)cc21] |
| 82 | 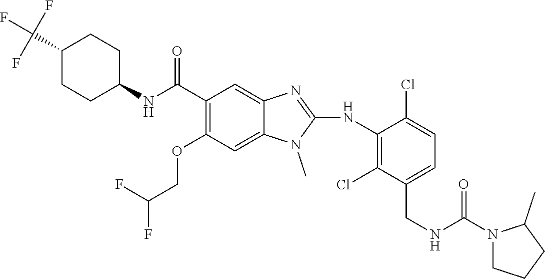 [SMILES: CC1CCCN1C(=O)NCc1ccc(Cl)c(Nc2nc3cc(C(=O)N[C@H]4CC[C@H](C(F)(F)F)CC4)c(OCC(F)F)cc3n2C)c1Cl] |
| 83 | 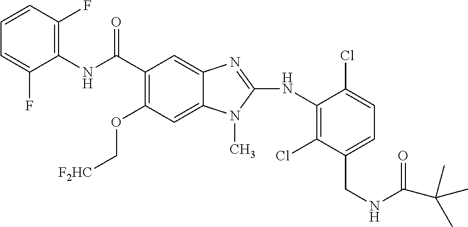 [SMILES: Cn1c(Nc2c(Cl)ccc(CNC(=O)C(C)(C)C)c2Cl)nc2cc(C(=O)Nc3c(F)cccc3F)c(OCC(F)F)cc21] |
| 84 | 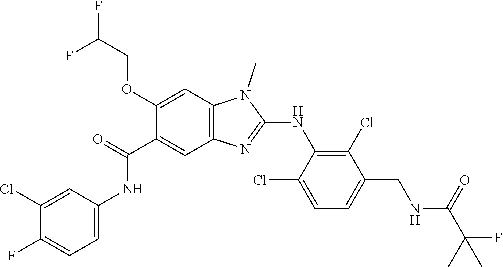 [SMILES: Cn1c(Nc2c(Cl)ccc(CNC(=O)C(C)(C)F)c2Cl)nc2cc(C(=O)Nc3ccc(F)c(Cl)c3)c(OCC(F)F)cc21] |
| 85 | 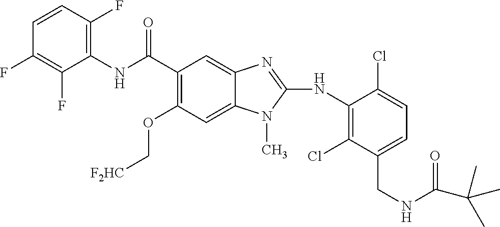 [SMILES: Cn1c(Nc2c(Cl)ccc(CNC(=O)C(C)(C)C)c2Cl)nc2cc(C(=O)Nc3c(F)ccc(F)c3F)c(OCC(F)F)cc21] |
| 86 | 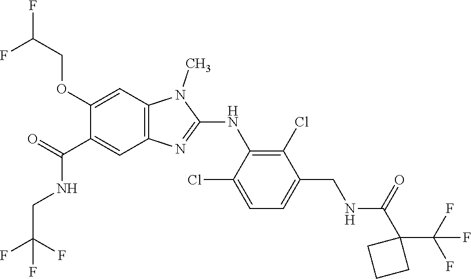 [SMILES: Cn1c(Nc2c(Cl)ccc(CNC(=O)C3(C(F)(F)F)CCC3)c2Cl)nc2cc(C(=O)NCC(F)(F)F)c(OCC(F)F)cc21] |
| 87 | 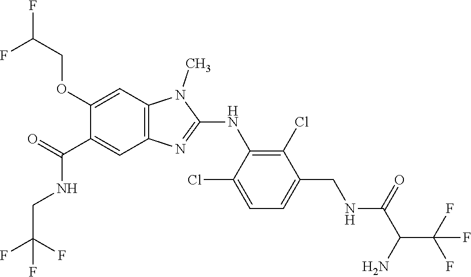 [SMILES: Cn1c(Nc2c(Cl)ccc(CNC(=O)C(N)C(F)(F)F)c2Cl)nc2cc(C(=O)NCC(F)(F)F)c(OCC(F)F)cc21] |
| 88 | 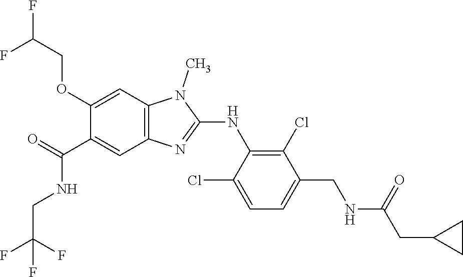 [SMILES: Cn1c(Nc2c(Cl)ccc(CNC(=O)CC3CC3)c2Cl)nc2cc(C(=O)NCC(F)(F)F)c(OCC(F)F)cc21] |
| 89 | 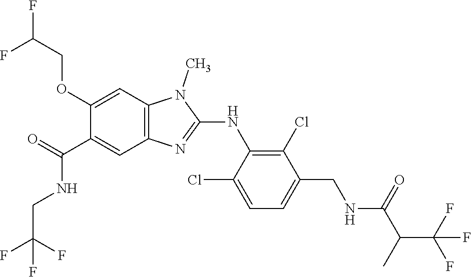 [SMILES: CC(C(=O)NCc1ccc(Cl)c(Nc2nc3cc(C(=O)NCC(F)(F)F)c(OCC(F)F)cc3n2C)c1Cl)C(F)(F)F] |
| 90 | 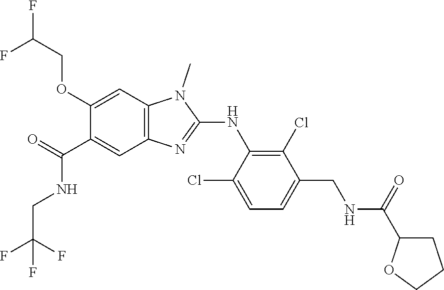 [SMILES: Cn1c(Nc2c(Cl)ccc(CNC(=O)C3CCCO3)c2Cl)nc2cc(C(=O)NCC(F)(F)F)c(OCC(F)F)cc21] |
| 91 | 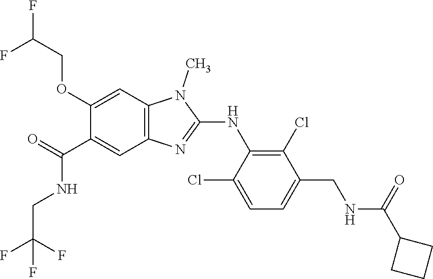 [SMILES: Cn1c(Nc2c(Cl)ccc(CNC(=O)C3CCC3)c2Cl)nc2cc(C(=O)NCC(F)(F)F)c(OCC(F)F)cc21] |
| 92 | 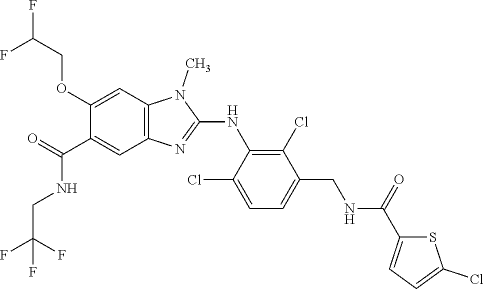 [SMILES: Cn1c(Nc2c(Cl)ccc(CNC(=O)c3ccc(Cl)s3)c2Cl)nc2cc(C(=O)NCC(F)(F)F)c(OCC(F)F)cc21] |
| 93 | 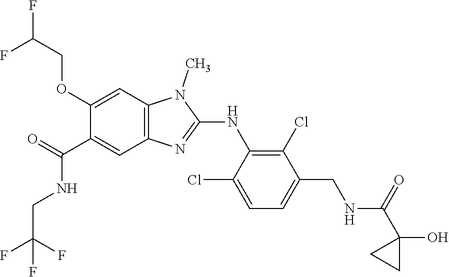 [SMILES: Cn1c(Nc2c(Cl)ccc(CNC(=O)C3(O)CC3)c2Cl)nc2cc(C(=O)NCC(F)(F)F)c(OCC(F)F)cc21] |
| 94 | 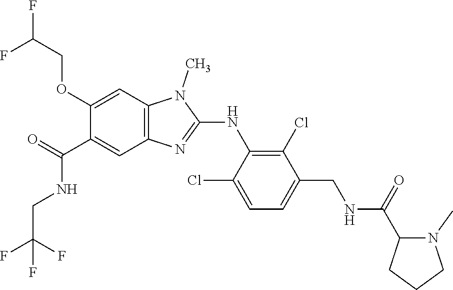 [SMILES: CN1CCCC1C(=O)NCc1ccc(Cl)c(Nc2nc3cc(C(=O)NCC(F)(F)F)c(OCC(F)F)cc3n2C)c1Cl] |
| 95 | 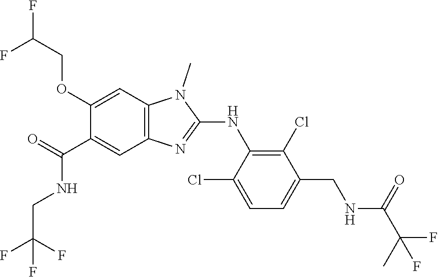 [SMILES: Cn1c(Nc2c(Cl)ccc(CNC(=O)C(C)(F)F)c2Cl)nc2cc(C(=O)NCC(F)(F)F)c(OCC(F)F)cc21] |
| 96 | 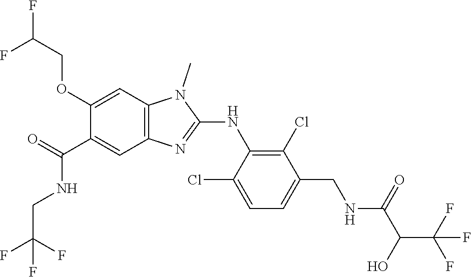 [SMILES: Cn1c(Nc2c(Cl)ccc(CNC(=O)C(O)C(F)(F)F)c2Cl)nc2cc(C(=O)NCC(F)(F)F)c(OCC(F)F)cc21] |
| 97 | 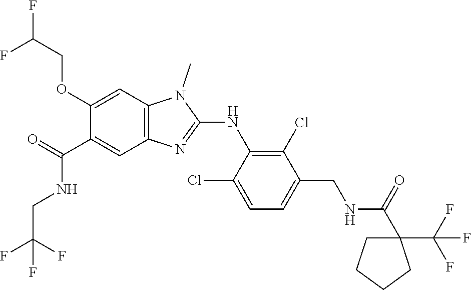 [SMILES: Cn1c(Nc2c(Cl)ccc(CNC(=O)C3(C(F)(F)F)CCCC3)c2Cl)nc2cc(C(=O)NCC(F)(F)F)c(OCC(F)F)cc21] |
| 98 | 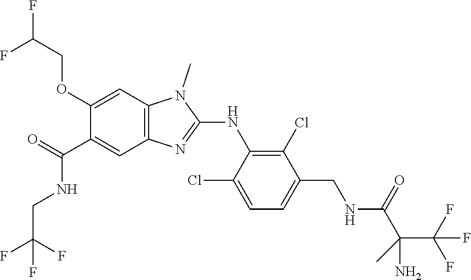 [SMILES: Cn1c(Nc2c(Cl)ccc(CNC(=O)C(C)(N)C(F)(F)F)c2Cl)nc2cc(C(=O)NCC(F)(F)F)c(OCC(F)F)cc21] |
| 99 | 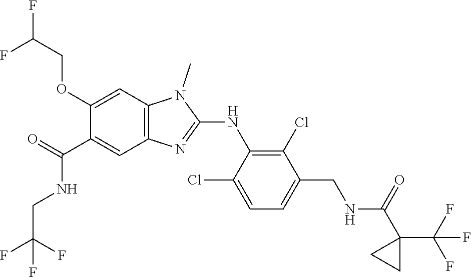 [SMILES: Cn1c(Nc2c(Cl)ccc(CNC(=O)C3(C(F)(F)F)CC3)c2Cl)nc2cc(C(=O)NCC(F)(F)F)c(OCC(F)F)cc21] |
| 100 | 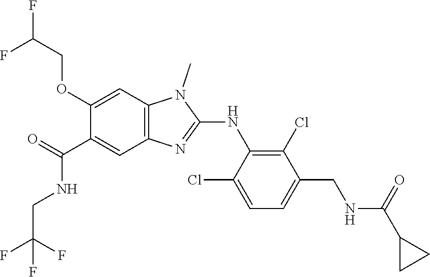 [SMILES: Cn1c(Nc2c(Cl)ccc(CNC(=O)C3CC3)c2Cl)nc2cc(C(=O)NCC(F)(F)F)c(OCC(F)F)cc21] |
| 101 | 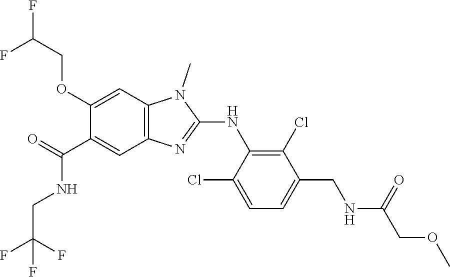 [SMILES: COCC(=O)NCc1ccc(Cl)c(Nc2nc3cc(C(=O)NCC(F)(F)F)c(OCC(F)F)cc3n2C)c1Cl] |
| 102 | 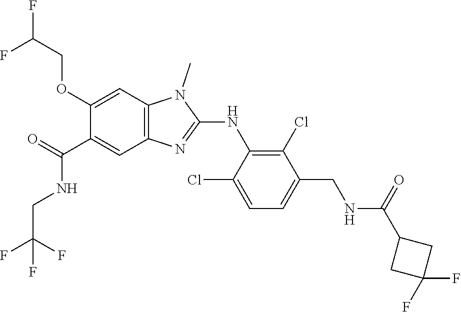 [SMILES: Cn1c(Nc2c(Cl)ccc(CNC(=O)C3CC(F)(F)C3)c2Cl)nc2cc(C(=O)NCC(F)(F)F)c(OCC(F)F)cc21] |
| 103 | 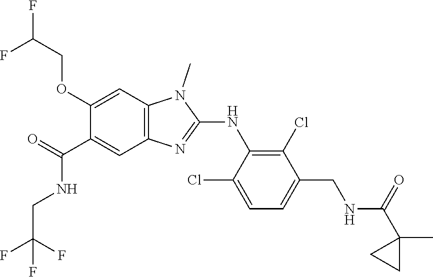 [SMILES: Cn1c(Nc2c(Cl)ccc(CNC(=O)C3(C)CC3)c2Cl)nc2cc(C(=O)NCC(F)(F)F)c(OCC(F)F)cc21] |
| 104 | 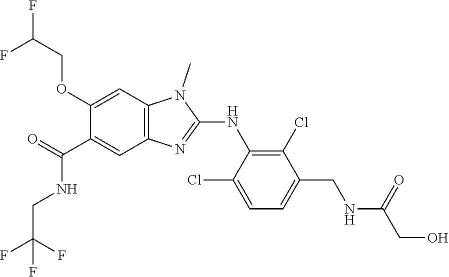 [SMILES: Cn1c(Nc2c(Cl)ccc(CNC(=O)CO)c2Cl)nc2cc(C(=O)NCC(F)(F)F)c(OCC(F)F)cc21] |
| 105 | 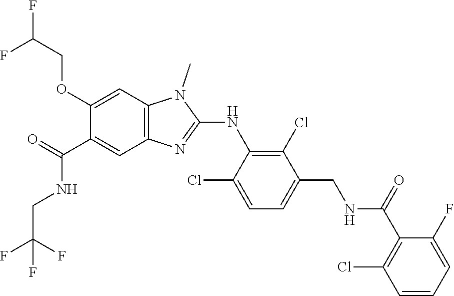 [SMILES: Cn1c(Nc2c(Cl)ccc(CNC(=O)c3c(F)cccc3Cl)c2Cl)nc2cc(C(=O)NCC(F)(F)F)c(OCC(F)F)cc21] |
| 106 | 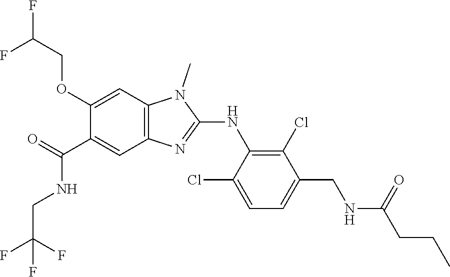 [SMILES: CCCC(=O)NCc1ccc(Cl)c(Nc2nc3cc(C(=O)NCC(F)(F)F)c(OCC(F)F)cc3n2C)c1Cl] |
| 107 | 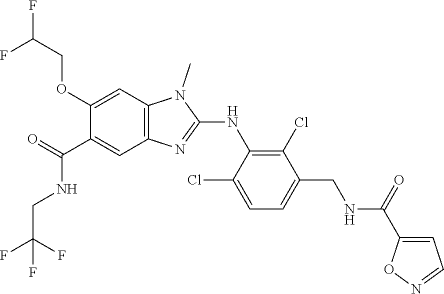 [SMILES: Cn1c(Nc2c(Cl)ccc(CNC(=O)c3ccno3)c2Cl)nc2cc(C(=O)NCC(F)(F)F)c(OCC(F)F)cc21] |
| 108 | 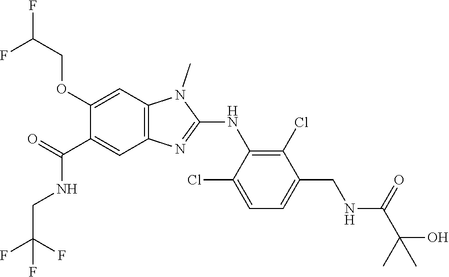 [SMILES: Cn1c(Nc2c(Cl)ccc(CNC(=O)C(C)(C)O)c2Cl)nc2cc(C(=O)NCC(F)(F)F)c(OCC(F)F)cc21] |
| 109 | 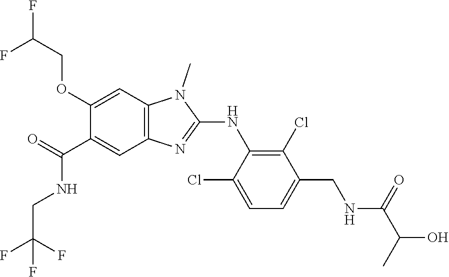 [SMILES: CC(O)C(=O)NCc1ccc(Cl)c(Nc2nc3cc(C(=O)NCC(F)(F)F)c(OCC(F)F)cc3n2C)c1Cl] |
| 110 | 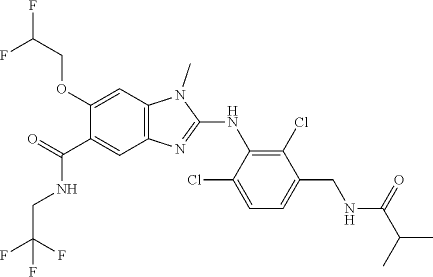 [SMILES: CC(C)C(=O)NCc1ccc(Cl)c(Nc2nc3cc(C(=O)NCC(F)(F)F)c(OCC(F)F)cc3n2C)c1Cl] |
| 111 | 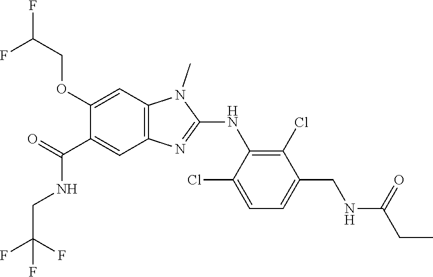 [SMILES: CCC(=O)NCc1ccc(Cl)c(Nc2nc3cc(C(=O)NCC(F)(F)F)c(OCC(F)F)cc3n2C)c1Cl] |
| 112 | 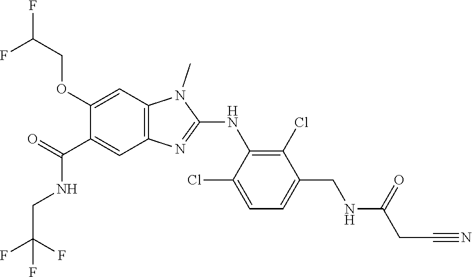 [SMILES: Cn1c(Nc2c(Cl)ccc(CNC(=O)CC#N)c2Cl)nc2cc(C(=O)NCC(F)(F)F)c(OCC(F)F)cc21] |
| 113 | 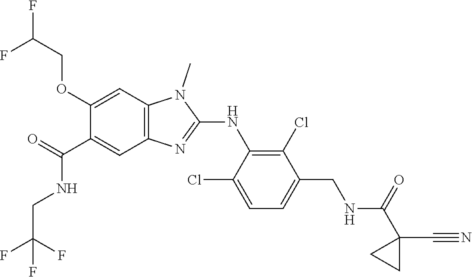 [SMILES: Cn1c(Nc2c(Cl)ccc(CNC(=O)C3(C#N)CC3)c2Cl)nc2cc(C(=O)NCC(F)(F)F)c(OCC(F)F)cc21] |
| 114 | 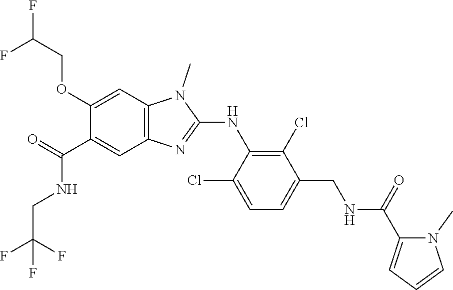 [SMILES: Cn1cccc1C(=O)NCc1ccc(Cl)c(Nc2nc3cc(C(=O)NCC(F)(F)F)c(OCC(F)F)cc3n2C)c1Cl] |
| 115 | 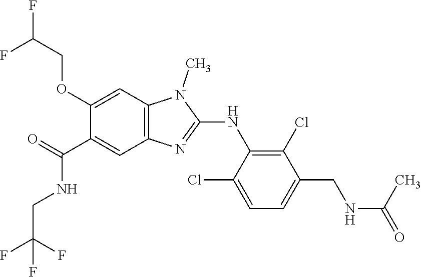 [SMILES: CC(=O)NCc1ccc(Cl)c(Nc2nc3cc(C(=O)NCC(F)(F)F)c(OCC(F)F)cc3n2C)c1Cl] |
| 116 | 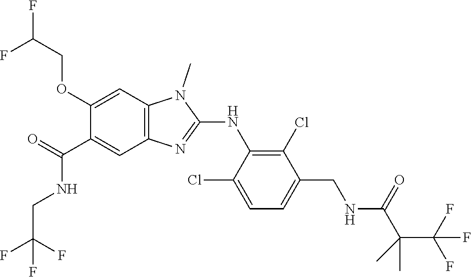 [SMILES: Cn1c(Nc2c(Cl)ccc(CNC(=O)C(C)(C)C(F)(F)F)c2Cl)nc2cc(C(=O)NCC(F)(F)F)c(OCC(F)F)cc21] |
| 117 | 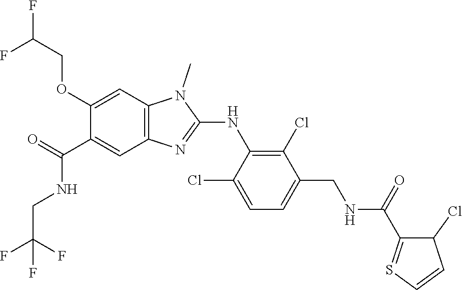 |
| 118 | 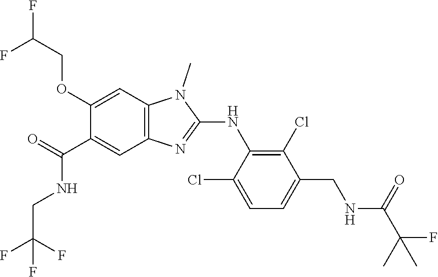 [SMILES: Cn1c(Nc2c(Cl)ccc(CNC(=O)C(C)(C)F)c2Cl)nc2cc(C(=O)NCC(F)(F)F)c(OCC(F)F)cc21] |
| 119 | 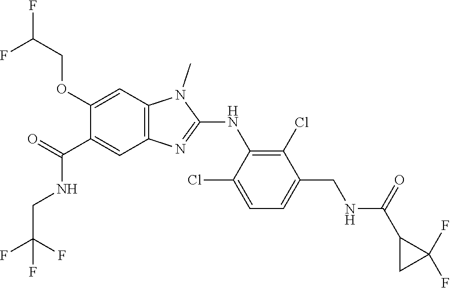 [SMILES: Cn1c(Nc2c(Cl)ccc(CNC(=O)C3CC3(F)F)c2Cl)nc2cc(C(=O)NCC(F)(F)F)c(OCC(F)F)cc21] |
| 120 | 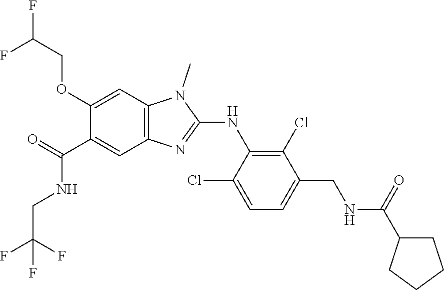 [SMILES: Cn1c(Nc2c(Cl)ccc(CNC(=O)C3CCCC3)c2Cl)nc2cc(C(=O)NCC(F)(F)F)c(OCC(F)F)cc21] |
| 121 | 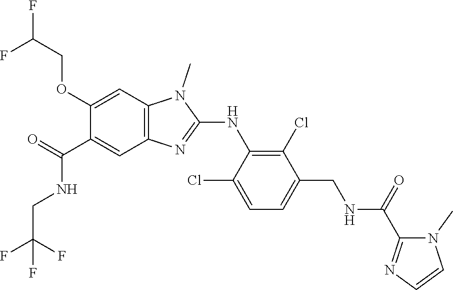 [SMILES: Cn1ccnc1C(=O)NCc1ccc(Cl)c(Nc2nc3cc(C(=O)NCC(F)(F)F)c(OCC(F)F)cc3n2C)c1Cl] |
| 122 | 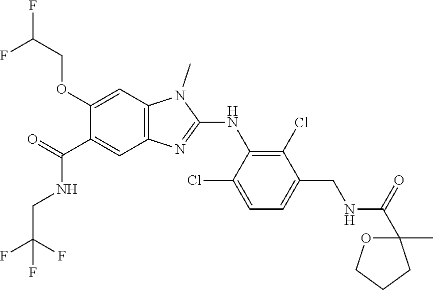 [SMILES: Cn1c(Nc2c(Cl)ccc(CNC(=O)C3(C)CCCO3)c2Cl)nc2cc(C(=O)NCC(F)(F)F)c(OCC(F)F)cc21] |
| 123 | 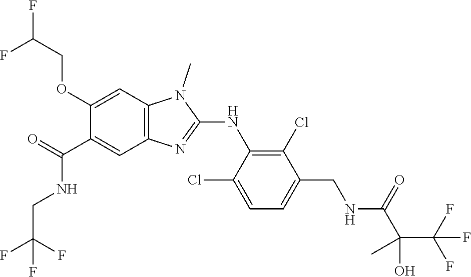 [SMILES: Cn1c(Nc2c(Cl)ccc(CNC(=O)C(C)(O)C(F)(F)F)c2Cl)nc2cc(C(=O)NCC(F)(F)F)c(OCC(F)F)cc21] |
| 124 | 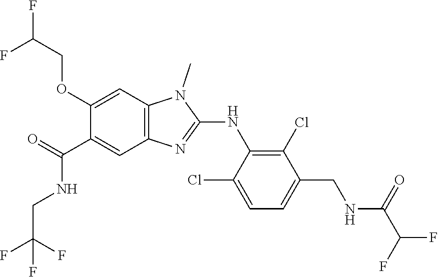 [SMILES: Cn1c(Nc2c(Cl)ccc(CNC(=O)C(F)F)c2Cl)nc2cc(C(=O)NCC(F)(F)F)c(OCC(F)F)cc21] |
| 125 | 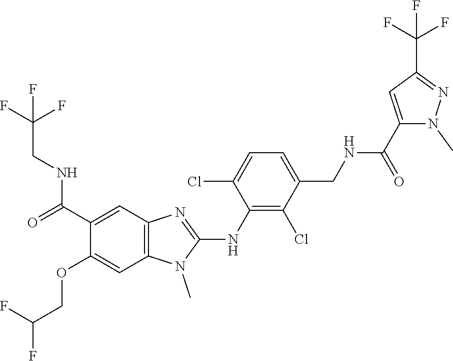 [SMILES: Cn1nc(C(F)(F)F)cc1C(=O)NCc1ccc(Cl)c(Nc2nc3cc(C(=O)NCC(F)(F)F)c(OCC(F)F)cc3n2C)c1Cl] |
| 126 | 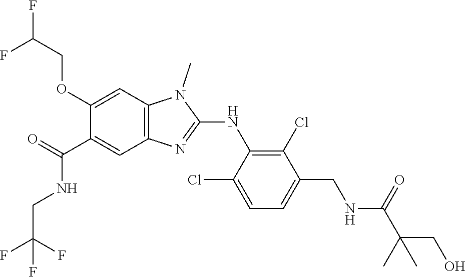 [SMILES: Cn1c(Nc2c(Cl)ccc(CNC(=O)C(C)(C)CO)c2Cl)nc2cc(C(=O)NCC(F)(F)F)c(OCC(F)F)cc21] |
| 127 | 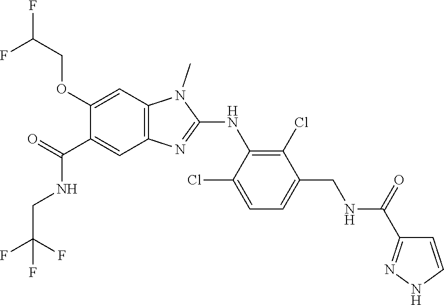 [SMILES: Cn1c(Nc2c(Cl)ccc(CNC(=O)c3cc[nH]n3)c2Cl)nc2cc(C(=O)NCC(F)(F)F)c(OCC(F)F)cc21] |
| 128 | 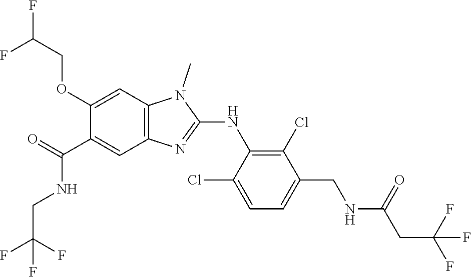 [SMILES: Cn1c(Nc2c(Cl)ccc(CNC(=O)CC(F)(F)F)c2Cl)nc2cc(C(=O)NCC(F)(F)F)c(OCC(F)F)cc21] |
| 129 | 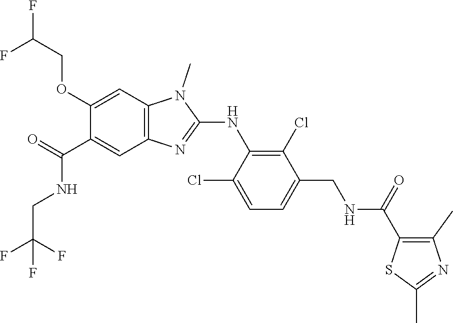 [SMILES: Cc1nc(C)c(C(=O)NCc2ccc(Cl)c(Nc3nc4cc(C(=O)NCC(F)(F)F)c(OCC(F)F)cc4n3C)c2Cl)s1] |
| 130 | 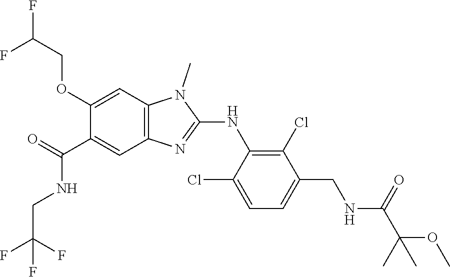 [SMILES: COC(C)(C)C(=O)NCc1ccc(Cl)c(Nc2nc3cc(C(=O)NCC(F)(F)F)c(OCC(F)F)cc3n2C)c1Cl] |
| 131 | 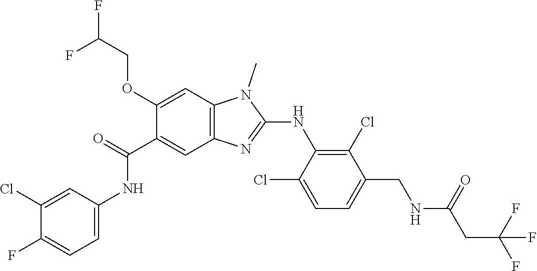 [SMILES: Cn1c(Nc2c(Cl)ccc(CNC(=O)CC(F)(F)F)c2Cl)nc2cc(C(=O)Nc3ccc(F)c(Cl)c3)c(OCC(F)F)cc21] |
| 132 | 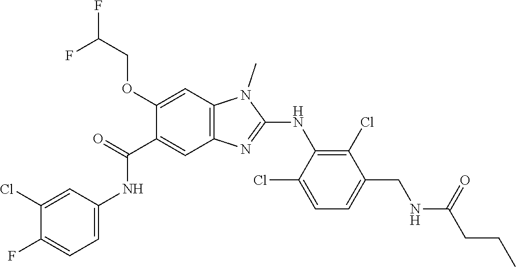 [SMILES: CCCC(=O)NCc1ccc(Cl)c(Nc2nc3cc(C(=O)Nc4ccc(F)c(Cl)c4)c(OCC(F)F)cc3n2C)c1Cl] |
| 133 | 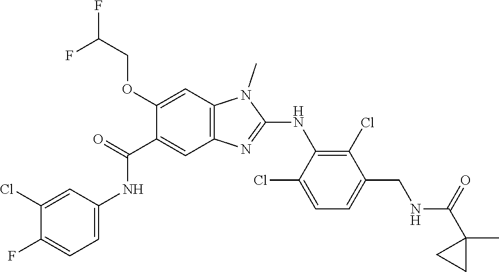 [SMILES: Cn1c(Nc2c(Cl)ccc(CNC(=O)C3(C)CC3)c2Cl)nc2cc(C(=O)Nc3ccc(F)c(Cl)c3)c(OCC(F)F)cc21] |
| 134 | 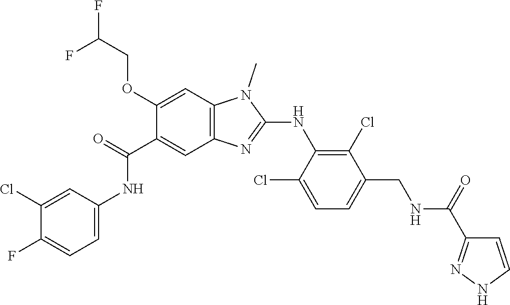 [SMILES: Cn1c(Nc2c(Cl)ccc(CNC(=O)c3cc[nH]n3)c2Cl)nc2cc(C(=O)Nc3ccc(F)c(Cl)c3)c(OCC(F)F)cc21] |
| 135 | 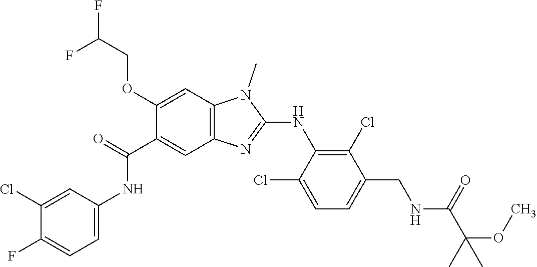 [SMILES: COC(C)(C)C(=O)NCc1ccc(Cl)c(Nc2nc3cc(C(=O)Nc4ccc(F)c(Cl)c4)c(OCC(F)F)cc3n2C)c1Cl] |
| 136 | 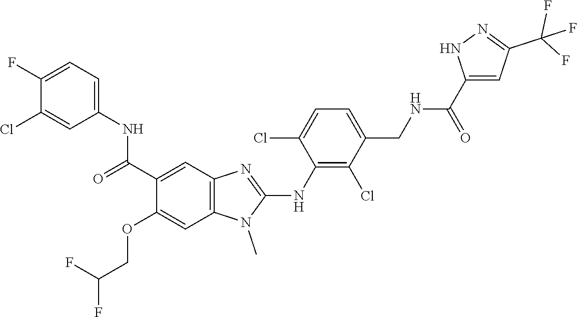 [SMILES: Cn1c(Nc2c(Cl)ccc(CNC(=O)c3cc(C(F)(F)F)n[nH]3)c2Cl)nc2cc(C(=O)Nc3ccc(F)c(Cl)c3)c(OCC(F)F)cc21] |
| 137 | 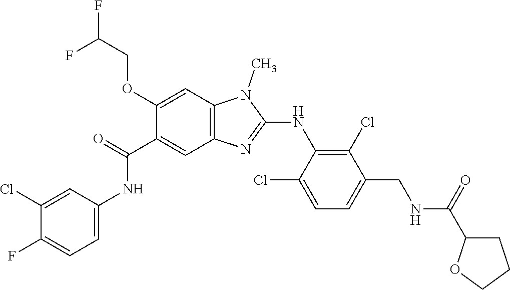 [SMILES: Cn1c(Nc2c(Cl)ccc(CNC(=O)C3CCCO3)c2Cl)nc2cc(C(=O)Nc3ccc(F)c(Cl)c3)c(OCC(F)F)cc21] |
| 138 | 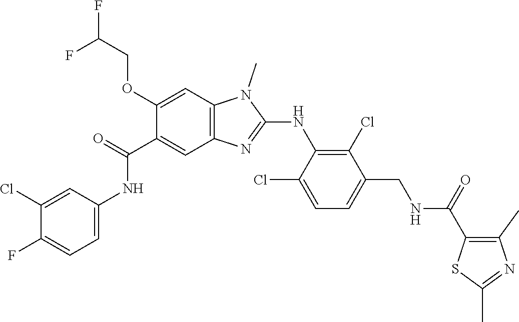 [SMILES: Cc1nc(C)c(C(=O)NCc2ccc(Cl)c(Nc3nc4cc(C(=O)Nc5ccc(F)c(Cl)c5)c(OCC(F)F)cc4n3C)c2Cl)s1] |
| 139 | 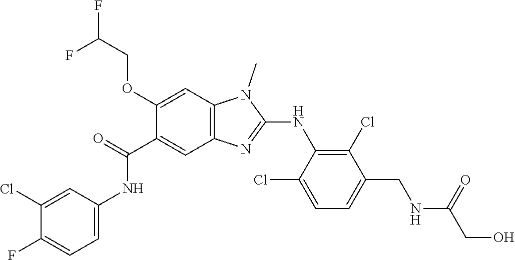 [SMILES: Cn1c(Nc2c(Cl)ccc(CNC(=O)CO)c2Cl)nc2cc(C(=O)Nc3ccc(F)c(Cl)c3)c(OCC(F)F)cc21] |
| 140 | 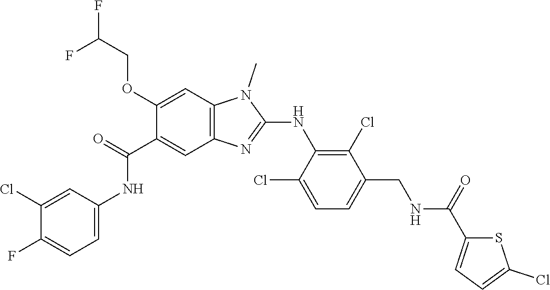 [SMILES: Cn1c(Nc2c(Cl)ccc(CNC(=O)c3ccc(Cl)s3)c2Cl)nc2cc(C(=O)Nc3ccc(F)c(Cl)c3)c(OCC(F)F)cc21] |
| 141 | 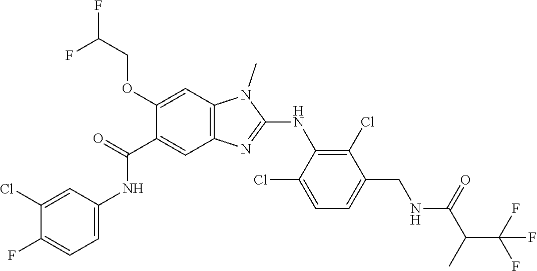 [SMILES: CC(C(=O)NCc1ccc(Cl)c(Nc2nc3cc(C(=O)Nc4ccc(F)c(Cl)c4)c(OCC(F)F)cc3n2C)c1Cl)C(F)(F)F] |
| 142 | 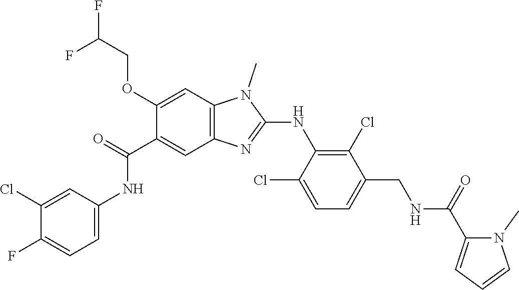 [SMILES: Cn1cccc1C(=O)NCc1ccc(Cl)c(Nc2nc3cc(C(=O)Nc4ccc(F)c(Cl)c4)c(OCC(F)F)cc3n2C)c1Cl] |
| 143 | 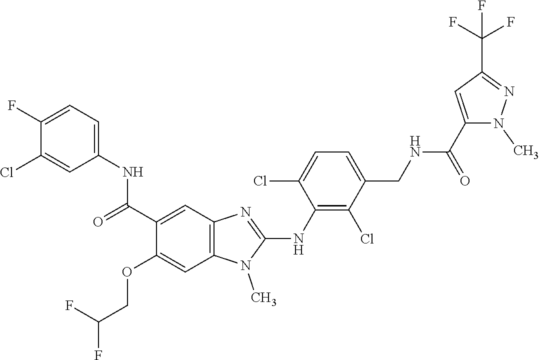 [SMILES: Cn1nc(C(F)(F)F)cc1C(=O)NCc1ccc(Cl)c(Nc2nc3cc(C(=O)Nc4ccc(F)c(Cl)c4)c(OCC(F)F)cc3n2C)c1Cl] |
| 144 | 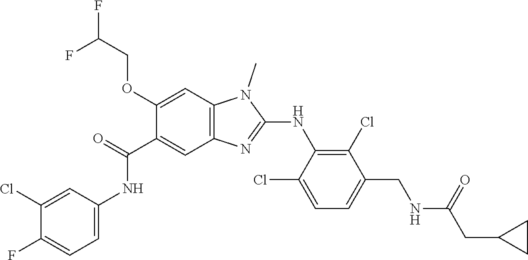 [SMILES: Cn1c(Nc2c(Cl)ccc(CNC(=O)CC3CC3)c2Cl)nc2cc(C(=O)Nc3ccc(F)c(Cl)c3)c(OCC(F)F)cc21] |
| 145 | 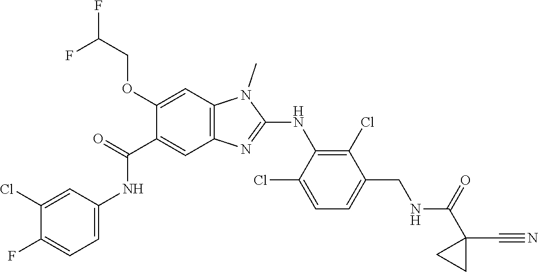 [SMILES: Cn1c(Nc2c(Cl)ccc(CNC(=O)C3(C#N)CC3)c2Cl)nc2cc(C(=O)Nc3ccc(F)c(Cl)c3)c(OCC(F)F)cc21] |
| 146 | 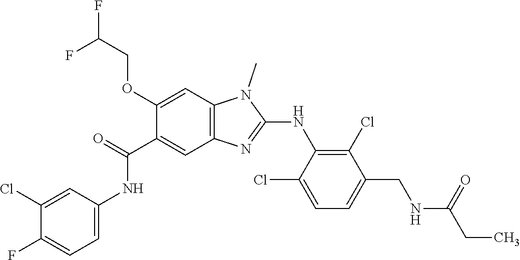 [SMILES: CCC(=O)NCc1ccc(Cl)c(Nc2nc3cc(C(=O)Nc4ccc(F)c(Cl)c4)c(OCC(F)F)cc3n2C)c1Cl] |
| 147 | 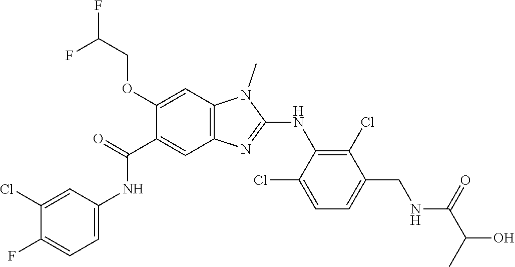 [SMILES: CC(O)C(=O)NCc1ccc(Cl)c(Nc2nc3cc(C(=O)Nc4ccc(F)c(Cl)c4)c(OCC(F)F)cc3n2C)c1Cl] |
| 148 | 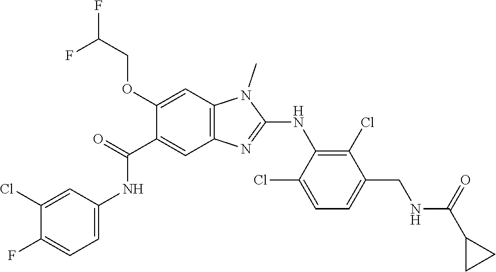 [SMILES: Cn1c(Nc2c(Cl)ccc(CNC(=O)C3CC3)c2Cl)nc2cc(C(=O)Nc3ccc(F)c(Cl)c3)c(OCC(F)F)cc21] |
| 149 | 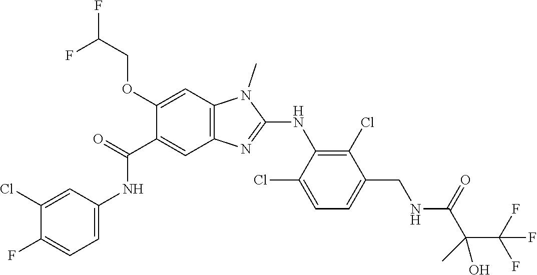 [SMILES: Cn1c(Nc2c(Cl)ccc(CNC(=O)C(C)(O)C(F)(F)F)c2Cl)nc2cc(C(=O)Nc3ccc(F)c(Cl)c3)c(OCC(F)F)cc21] |
| 150 | 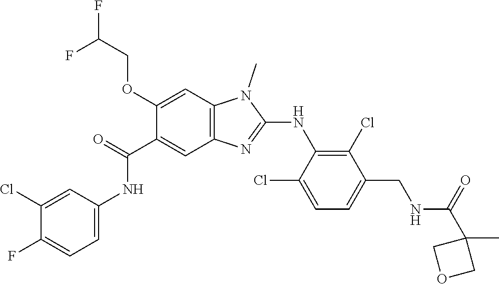 [SMILES: Cn1c(Nc2c(Cl)ccc(CNC(=O)C3(C)COC3)c2Cl)nc2cc(C(=O)Nc3ccc(F)c(Cl)c3)c(OCC(F)F)cc21] |
| 151 | 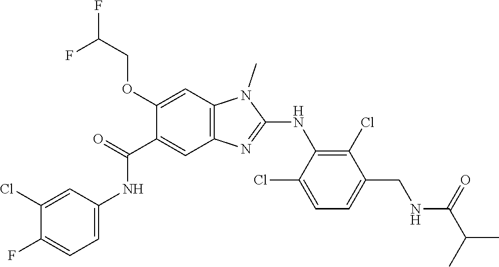 [SMILES: CC(C)C(=O)NCc1ccc(Cl)c(Nc2nc3cc(C(=O)Nc4ccc(F)c(Cl)c4)c(OCC(F)F)cc3n2C)c1Cl] |
| 152 | 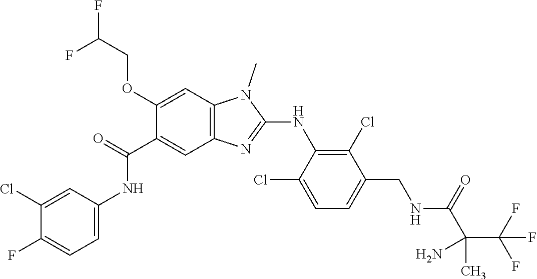 [SMILES: Cn1c(Nc2c(Cl)ccc(CNC(=O)C(C)(N)C(F)(F)F)c2Cl)nc2cc(C(=O)Nc3ccc(F)c(Cl)c3)c(OCC(F)F)cc21] |
| 153 | 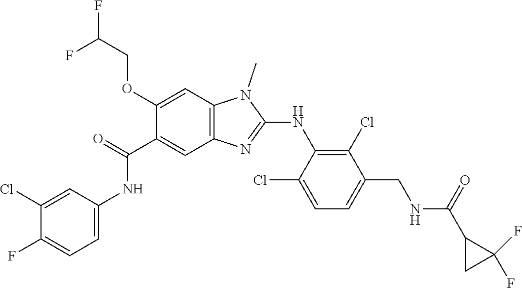 [SMILES: Cn1c(Nc2c(Cl)ccc(CNC(=O)C3CC3(F)F)c2Cl)nc2cc(C(=O)Nc3ccc(F)c(Cl)c3)c(OCC(F)F)cc21] |
| 154 | 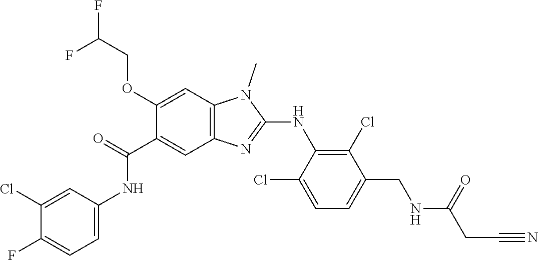 [SMILES: Cn1c(Nc2c(Cl)ccc(CNC(=O)CC#N)c2Cl)nc2cc(C(=O)Nc3ccc(F)c(Cl)c3)c(OCC(F)F)cc21] |
| 155 | 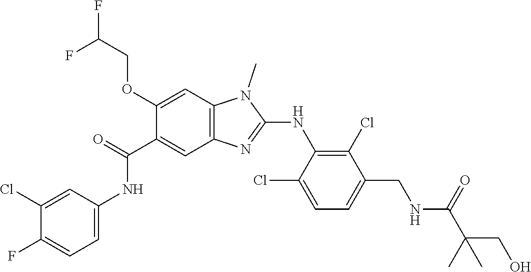 [SMILES: Cn1c(Nc2c(Cl)ccc(CNC(=O)C(C)(C)CO)c2Cl)nc2cc(C(=O)Nc3ccc(F)c(Cl)c3)c(OCC(F)F)cc21] |
| 156 | 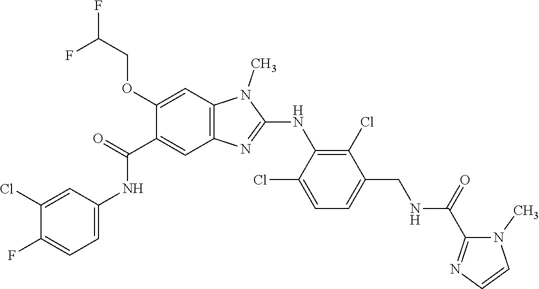 [SMILES: Cn1ccnc1C(=O)NCc1ccc(Cl)c(Nc2nc3cc(C(=O)Nc4ccc(F)c(Cl)c4)c(OCC(F)F)cc3n2C)c1Cl] |
| 157 | 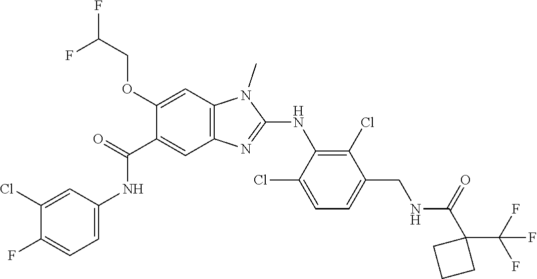 [SMILES: Cn1c(Nc2c(Cl)ccc(CNC(=O)C3(C(F)(F)F)CCC3)c2Cl)nc2cc(C(=O)Nc3ccc(F)c(Cl)c3)c(OCC(F)F)cc21] |
| 158 | 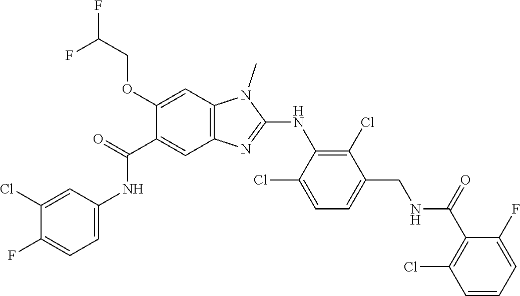 [SMILES: Cn1c(Nc2c(Cl)ccc(CNC(=O)c3c(F)cccc3Cl)c2Cl)nc2cc(C(=O)Nc3ccc(F)c(Cl)c3)c(OCC(F)F)cc21] |
| 159 | 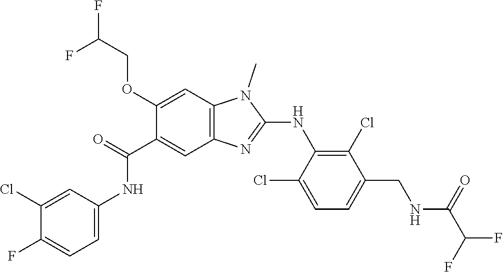 [SMILES: Cn1c(Nc2c(Cl)ccc(CNC(=O)C(F)F)c2Cl)nc2cc(C(=O)Nc3ccc(F)c(Cl)c3)c(OCC(F)F)cc21] |
| 160 | 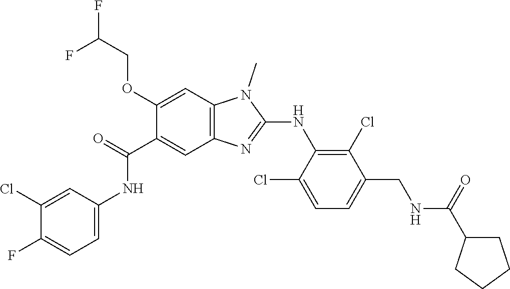 [SMILES: Cn1c(Nc2c(Cl)ccc(CNC(=O)C3CCCC3)c2Cl)nc2cc(C(=O)Nc3ccc(F)c(Cl)c3)c(OCC(F)F)cc21] |
| 161 | 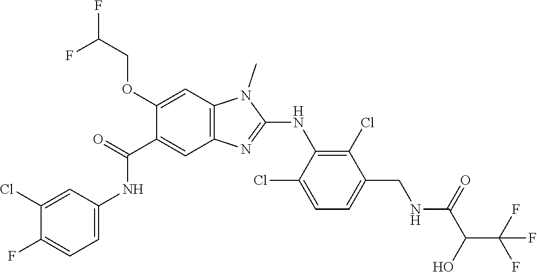 [SMILES: Cn1c(Nc2c(Cl)ccc(CNC(=O)C(O)C(F)(F)F)c2Cl)nc2cc(C(=O)Nc3ccc(F)c(Cl)c3)c(OCC(F)F)cc21] |
| 162 | 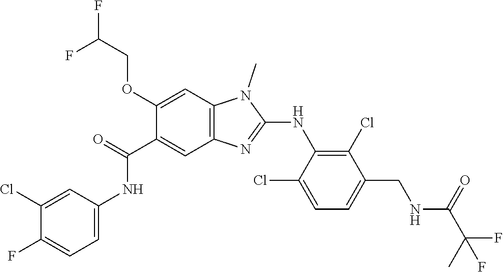 [SMILES: Cn1c(Nc2c(Cl)ccc(CNC(=O)C(C)(F)F)c2Cl)nc2cc(C(=O)Nc3ccc(F)c(Cl)c3)c(OCC(F)F)cc21] |
| 163 | 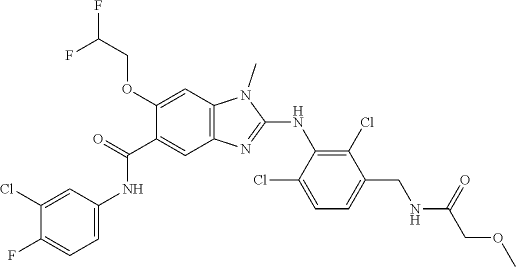 [SMILES: COCC(=O)NCc1ccc(Cl)c(Nc2nc3cc(C(=O)Nc4ccc(F)c(Cl)c4)c(OCC(F)F)cc3n2C)c1Cl] |
| 164 | 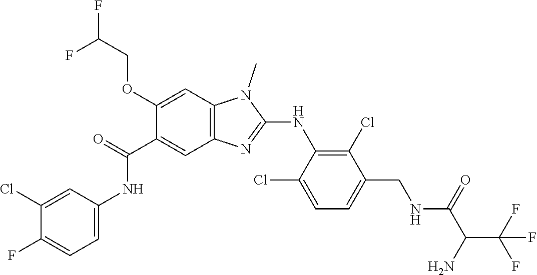 [SMILES: Cn1c(Nc2c(Cl)ccc(CNC(=O)C(N)C(F)(F)F)c2Cl)nc2cc(C(=O)Nc3ccc(F)c(Cl)c3)c(OCC(F)F)cc21] |
| 165 | 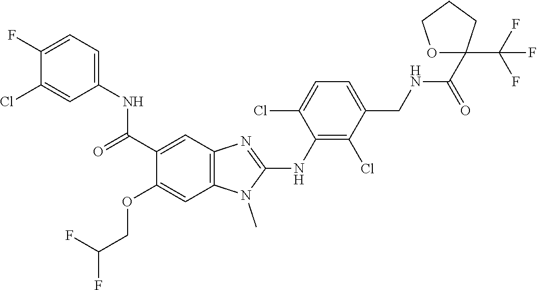 [SMILES: Cn1c(Nc2c(Cl)ccc(CNC(=O)C3(C(F)(F)F)CCCO3)c2Cl)nc2cc(C(=O)Nc3ccc(F)c(Cl)c3)c(OCC(F)F)cc21] |
| 166 | 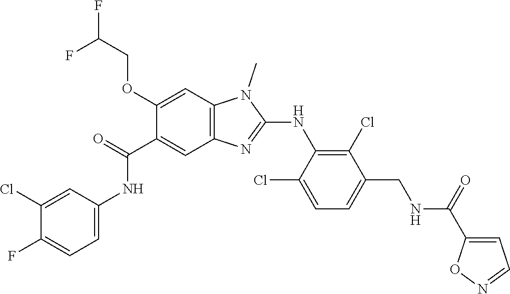 [SMILES: Cn1c(Nc2c(Cl)ccc(CNC(=O)c3ccno3)c2Cl)nc2cc(C(=O)Nc3ccc(F)c(Cl)c3)c(OCC(F)F)cc21] |
| 167 | 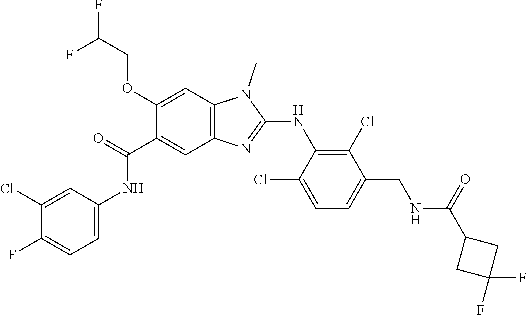 [SMILES: Cn1c(Nc2c(Cl)ccc(CNC(=O)C3CC(F)(F)C3)c2Cl)nc2cc(C(=O)Nc3ccc(F)c(Cl)c3)c(OCC(F)F)cc21] |
| 168 | 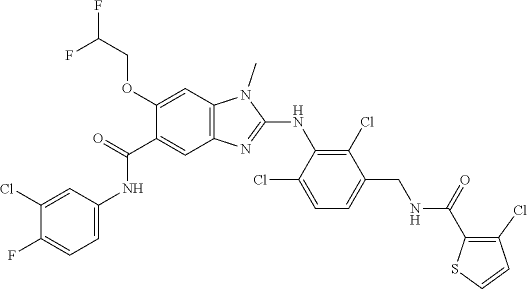 [SMILES: Cn1c(Nc2c(Cl)ccc(CNC(=O)c3sccc3Cl)c2Cl)nc2cc(C(=O)Nc3ccc(F)c(Cl)c3)c(OCC(F)F)cc21] |
| 169 | 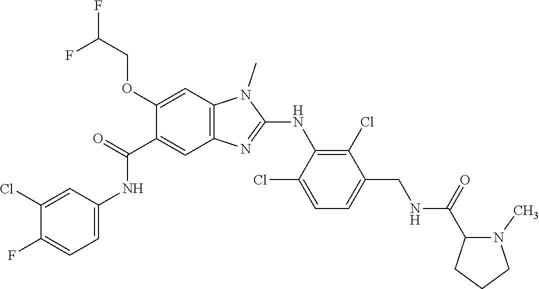 [SMILES: CN1CCCC1C(=O)NCc1ccc(Cl)c(Nc2nc3cc(C(=O)Nc4ccc(F)c(Cl)c4)c(OCC(F)F)cc3n2C)c1Cl] |
| 170 | 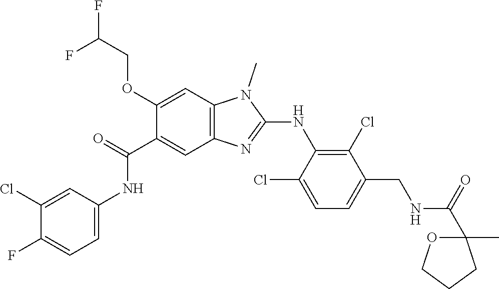 [SMILES: Cn1c(Nc2c(Cl)ccc(CNC(=O)C3(C)CCCO3)c2Cl)nc2cc(C(=O)Nc3ccc(F)c(Cl)c3)c(OCC(F)F)cc21] |
| 171 | 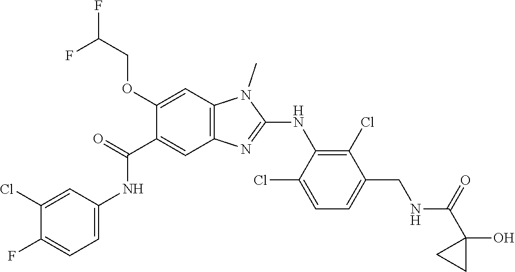 [SMILES: Cn1c(Nc2c(Cl)ccc(CNC(=O)C3(O)CC3)c2Cl)nc2cc(C(=O)Nc3ccc(F)c(Cl)c3)c(OCC(F)F)cc21] |
| 172 | 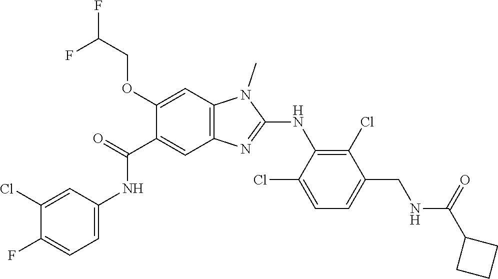 [SMILES: Cn1c(Nc2c(Cl)ccc(CNC(=O)C3CCC3)c2Cl)nc2cc(C(=O)Nc3ccc(F)c(Cl)c3)c(OCC(F)F)cc21] |
| 173 | 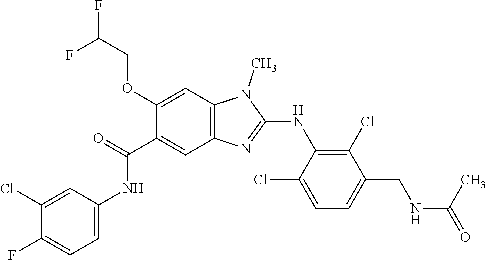 [SMILES: CC(=O)NCc1ccc(Cl)c(Nc2nc3cc(C(=O)Nc4ccc(F)c(Cl)c4)c(OCC(F)F)cc3n2C)c1Cl] |
| 174 | 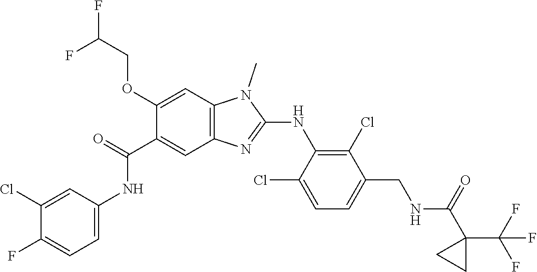 [SMILES: Cn1c(Nc2c(Cl)ccc(CNC(=O)C3(C(F)(F)F)CC3)c2Cl)nc2cc(C(=O)Nc3ccc(F)c(Cl)c3)c(OCC(F)F)cc21] |
| 175 | 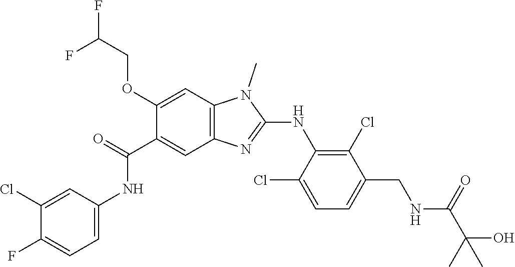 [SMILES: Cn1c(Nc2c(Cl)ccc(CNC(=O)C(C)(C)O)c2Cl)nc2cc(C(=O)Nc3ccc(F)c(Cl)c3)c(OCC(F)F)cc21] |
| 176 | 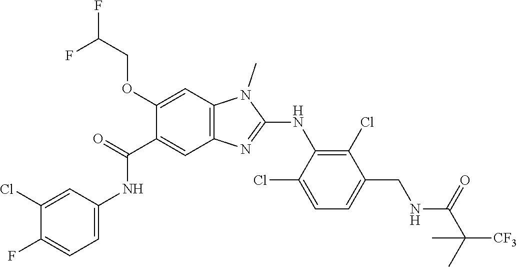 [SMILES: Cn1c(Nc2c(Cl)ccc(CNC(=O)C(C)(C)C(F)(F)F)c2Cl)nc2cc(C(=O)Nc3ccc(F)c(Cl)c3)c(OCC(F)F)cc21] |
| 177 | 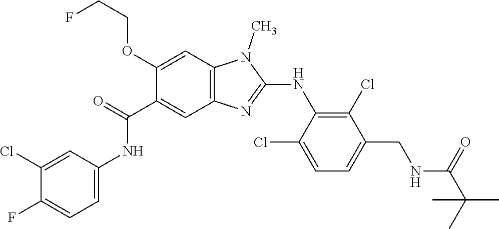 [SMILES: Cn1c(Nc2c(Cl)ccc(CNC(=O)C(C)(C)C)c2Cl)nc2cc(C(=O)Nc3ccc(F)c(Cl)c3)c(OCCF)cc21] |
| 178 | 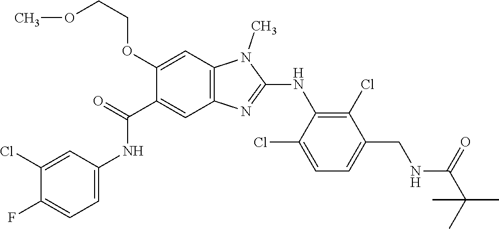 [SMILES: COCCOc1cc2c(cc1C(=O)Nc1ccc(F)c(Cl)c1)nc(Nc1c(Cl)ccc(CNC(=O)C(C)(C)C)c1Cl)n2C] |
| 179 | 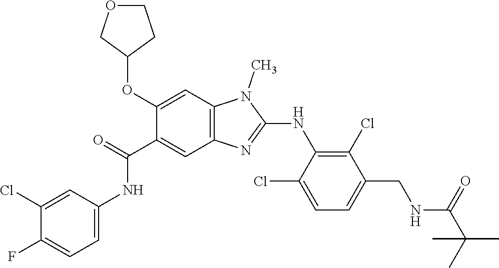 [SMILES: Cn1c(Nc2c(Cl)ccc(CNC(=O)C(C)(C)C)c2Cl)nc2cc(C(=O)Nc3ccc(F)c(Cl)c3)c(OC3CCOC3)cc21] |
| 180 | 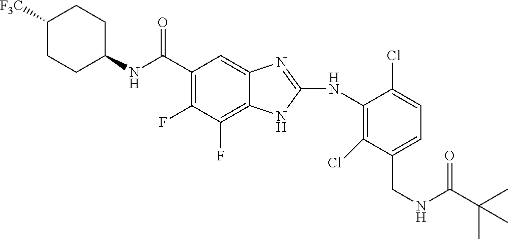 [SMILES: CC(C)(C)C(=O)NCc1ccc(Cl)c(Nc2nc3cc(C(=O)N[C@H]4CC[C@H](C(F)(F)F)CC4)c(F)c(F)c3[nH]2)c1Cl] |
| 181 | 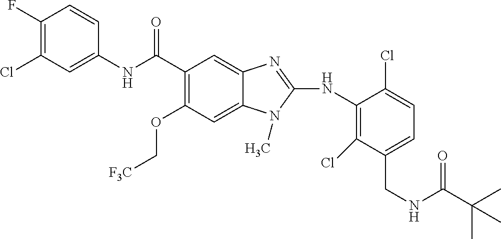 [SMILES: Cn1c(Nc2c(Cl)ccc(CNC(=O)C(C)(C)C)c2Cl)nc2cc(C(=O)Nc3ccc(F)c(Cl)c3)c(OCC(F)(F)F)cc21] |
| 182 | 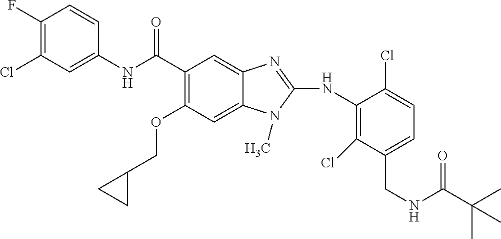 [SMILES: Cn1c(Nc2c(Cl)ccc(CNC(=O)C(C)(C)C)c2Cl)nc2cc(C(=O)Nc3ccc(F)c(Cl)c3)c(OCC3CC3)cc21] |

In yet further embodiments, compounds that may be utilised as the required mPGES-1 inhibitor may include the following compounds as set out in the list forming Group C below:
(R)-1-(5,6-dichloro-1-(quinolin-2-yl)-1H-benzo[d]imidazol-2-yl)-N-(tetrahydrofuran-3-yl)piperidine-4-carboxamide;
(R)-1-(5-chloro-6-methyl-1-(quinolin-2-yl)-1H-benzo[d]imidazol-2-yl)-N-(tetrahydrofurane-3-yl)piperidine-4-carboxamide;
(R)-1-(6-chloro-5-methyl-1-(quinolin-2-yl)-1H-benzo[d]imidazol-2-yl)-N-(tetrahydrofuran-3-yl)piperidine-4-carboxamide;
(R)-1-(5,6-dichloro-1-(6-chloroquinolin-2-yl)-1H-benzo[d]imidazol-2-yl)-N-(tetrahydrofuran-3-yl)piperidino-4-carboxamide;
(R)-1-(1-(6-chloroquinolin-2-yl)-5,6-dimethyl-1H-benzo[d]imidazol-2-yl)-N-(tetrahydrofuran-3-yl)piperidine-4-carboxamide;
(R)-1-(5,6-dichloro-1-(4-methylquinolin-2-yl)-1H-benzo[d]imidazol-2-yl)-N-(tetrahydrofuran-3-yl)piperidine-4-carboxamide;
(R)-1-(5-chloro-1-(6-chloroquinolin-2-yl)-6-methyl-H-benzo[d]imidazol-2-yl)-N-(tetrahydrofuran-3-yl)piperidine-4-carboxamide;
(R)-1-(6-chloro-1-(6-chloroquinolin-2-yl)-5-methyl-1H-benzo[d]imidazol-2-yl)-N-(tetrahydrofuran-3-yl)piperidine-4-carboxamide;
(R)-1-(5,6-dichloro-1-(6-fluoroquinolin-2-yl)-1H-benzo[d]imidazol-2-yl)-N-(tetrahydrofuran-3-yl)piperidine-4-carboxamide;
(R)-1-(5-chloro-1-(6-fluoroquinolin-2-yl)-6-methyl-1H-benzo[d]imidazol-2-yl)-N-(tetrahydrofuran-3-yl)piperidine-4-carboxamide;
(R)-1-(6-chloro-1-(6-fluoroquinolin-2-yl)-5-methyl-1H-benzo[d]imidazol-2-yl)-N-(tetrahydrofuran-3-yl)piperidine-4-carboxamide;
(R)-1-(5,6-dichloro-1-(8-fluoroquinolin-2-yl)-1H-benzo[d]imidazol-2-yl)-N-(tetrahydrofuran-3-yl)piperidine-4-carboxamide; and
(R)-1-(5,6-dichloro-1-(isoquinolin-3-yl)-H-benzo[d]imidazol-2-yl)-N-(tetrahydrofuran-3-yl)piperidine-4-carboxamide;
   or a pharmaceutically acceptable salt thereof.

In yet further embodiments, compounds that may be utilised as the required mPGES-1 inhibitor may include the following compounds as set out in the list forming Group D below;
A-cyclopentyl-1-(1-ethyl-5,6-dimethyl-1H-benzo[d]imidazol-2-yl)piperidine-4-carboxamide;
N-cyclopentyl-1-(5,6-dimethyl-1-propyl-1H-benzo[d]imidazol-2-yl)piperidine-4-carboxamide;
1-(1-butyl-5,6-dimethyl-1H-benzo[d]imidazol-2-yl)-N-cyclopentylpiperidine-4-carboxamide;
N-cyclopentyl-1-(1-heptyl-5,6-dimethyl-H-benzo[d]imidazol-2-yl)piperidine-4-carboxamide;
N-cyclopentyl-1-(5,6-dimethyl-1-octyl-1H-benzo[d]imidazol-2-yl)piperidine-4-carboxamide
N-cyclopentyl-1-(5,6-dimethyl-1-pentyl-1H-benzo[d]imidazol-2-yl)piperidine-4-carboxamide;
N-cyclopentyl-1-(1-hexyl-5,6-dimethyl-1H-benzo[d]imidazol-2-yl)piperidine-4-carboxamide;
N-cyclopentyl-1-(5,6-dimethyl-1-(pyridin-2-ylmethyl)-1H-benzo[d]imidazol-2-yl)piperidine-4-carboxamide;
N-cyclopentyl-1-(1-isopropyl-5,6-dimethyl-1H-benzo[d]imidazol-2-yl)piperidine-4-carboxamide;
N-cyclopentyl-1-(1-(2-(diethylamino)ethyl)-5,6-dichloro-1H-benzo[d]imidazol-2-yl)piperidine-4-carboxamide;
N-cyclopentyl-1-(5,6-dichloro-1-(pyridin-4-ylmethyl)-1H-benzo[d]imidazol-2-yl)piperidine-4-carboxamide;
N-Cyclopentyl-1-(5,6-dichloro-1-(pyridin-3-ylmethyl)-1H-benzo[d]imidazol-2-yl)piperidine-4-carboxamide;
N7-cyclopentyl-1-(5,6-dichloro-1-(pyridin-2-ylmethyl)-1H-benzo[d]imidazol-2-yl)piperidine-4-carboxamide;
1-(1-sec-butyl-5,6-dichloro-1H-benzo[d]imidazol-2-yl)-N-cyclopentylpiperidine-4-carboxamide;
N-cyclopentyl-1-(5,6-dichloro-1-(1-pentan-3-yl)-H-benzo[d]imidazol-2-yl)piperidine-4-carboxamide;
N-cyclopentyl-1-(5,6-dichloro-1-cyclobutyl-1H-benzo[d]imidazol-2-yl)piperidine-4-carboxamide;
N-cyclopentyl-1-(5,6-dichloro-1-(1-phenylethyl)-1H-benzo[d]imidazol-2-yl)piperidine-4-carboxamide;
N-cyclpentyl-1-(5,6-dichloro-1-methyl-1H-benzo[d]imidazol-2-yl)piperidine-4-carboxamide;
N-cyclopentyl-1-(5,6-dichloro-1-ethyl-1-benzo[d]imidazol-2-yl)piperidine-4-carboxamide;
N-cyclopentyl-1-(5,6-dichloro-1-isopropyl-1-benzo[d]imidazol-2-yl)piperidine-4-carboxamide;
N-cyclopentyl-1-(5,6-dichloro-1-(4-fluorobenzyl)-1H-benzo[d]imidazol-2-yl)piperidine-4-carboxamide;
N-cyclopentyl-1-(1-(3,5-difluorobenzyl)-5,6-dimethyl-1H-benzo[d]imidazol-2-yl)piperidine-4-carboxamide;
methyl 4-((2-(4-(cyclo pentylcarbamoyl)piperidin-1-yl)-5,6-dimethyl-1H-benzo[d]imidazol-1-yl)methyl)benzoate;
N-cyclopentyl-1-(5,6-dimethyl-1-((2-methythiazole-4-yl)methyl)-1H-benzo[d]imidazol-2-yl)piperidine-4-carboxamide;
N-cyclopentyl-1-(5,6-dichloro-1-phenyl-1H-benzo[d]imidazol-2-yl)piperidine-4-carboxamide;
N-cyclopropyl-1-(5,6-dichloro-1-isopropyl-H-benzo[d]imidazol-2-yl)piperidine-4-carboxamide;
N-cyclobutyl-1-(5,6-dichloro-1-isopropyl-1H-benzo[d]imidazol-2-yl)piperidine-4-carboxamide;
1-(5,6-dichloro-1-isopropyl-1H-benzo[d]imidazol-2-yl)-N-propylpiperidino-4-carboxamide;
1-(5,6-dichloro-1-isopopyl-1H-benzo[d]imidazol-2-yl)-N-(2-methoxyethyl)piperidine-4-carboxamide;
1-(5,6-dichloro-1-isopropyl-1H-benzo[d]imidazol-2-yl)-N-(isopropoxymethyl)piperidine-4-carboxamide;
1-(5,6-dichloro-1-isopropyl-1H-benzo[d]imidazol-2-yl)-N-(tetrahydrofuran-3-yl)piperidine-4-carboxamide;
N-(cyclohexylmethyl)-1-(5,6-dichloro-1-isopropyl-1H-benzo[d]imidazol-2-yl)piperidine-4-carboxamide;
1-(5,6-dichloro-1-isopropyl-1H-benzo[d]imidazol-2-yl)-N-((tetrahydro-2H-piperidine-4-yl)methyl)piperidine-4-carboxamide;
N-butyl-1-(5,6-dichloro-1-isopropyl-1H-benzo[d]imidazol-2-yl)piperidine-4-carboxamide;
N-(cyclopentylmethyl)-1-(5,6-dichloro-1-isopropyl-1H-benzo[d]imidazol-2-yl)piperidine-4-carboxamide;
N-cyclopentyl-1-(1-((6-fluoropyridin-3-yl)methyl)-5,6-dimethyl-1H-benzo[d]imidazol-2-yl)piperidine-4-carboxamide;
1-(1-(4-cyanobenzyl)-5,6-dimethyl-H-benzo[d]imidazol-2-yl)-N-cyclopentylpiperidine-4-carboxamide;
N-cyclopentyl-1-(1-(cyclopropylmethyl)-5,6-dimethyl-1H-benzo[d]imidazol-2-yl)piperidine-4-carboxamide;
N-cyclopentyl-1-(5,6-dimethyl-1-((tetrahydrofuran-2-yl)methyl)-1H-benzo[d]imidazol-2-yl)piperidine-4-carboxamide;
1-(1-(4-(1H-1,2,4-triazol-1-yl)benzyl)-5,6-dimethyl-1H-benzo[d]imidazol-2-yl)-N-cyclopentylpiperidine-4-carboxamide;
1-(1-cyclobutyl-5,6-dimethyl-H-benzo[d]imidazol-2-yl)-N-cyclopentylpiperidine-4-carboxamide;
1-(1-(4-bromobenzyl)-5,6-dimethyl-1H-benzo[d]imidazol-2-yl)-N-cyclopentylpiperidine-4-carboxamide;
N-cyclopentyl-1-(5,6-dimethyl-1-(4-(methylcarbamoyl)benzyl)-1H-benzo[d]imidazol-2-yl)piperidine-4-carboxamide;
N-cyclopentyl-1-(1-(4-(dimethylcarbamoyl)benzyl)-5,6-dimethyl-1H-benzo[d]imidazol-2-yl)piperidine-4-carboxamide;
1-(1-(4-carbamoylbenzyl)-5,6-dimethyl-1H-benzo[d]imidazol-2-yl)-N-cyclopentylpiperidine-4-carboxamide;
N-cyclopentyl-1-(5,6-dimethyl-1-(4-(methylsulfonylcarbamoyl)benzyl)-1H-benzo[d]imidazol-2-yl)piperidine-4-carboxamide;
1-(1-cyclobutyl-5,6-dimethyl-1H-benzo[d]imidazol-2-yl)-N-(tetrahydrofuran-3-yl)piperidine-4-carboxamide;
1-(1-(4-fluorobenzyl)-5,6-dimethyl-1H-benzo[d]imidazol-2-yl)-N-(tetrahydrofuran-3-yl)piperidine-4-carboxamide;
1-(5-chloro-1-cyclobutyl-6-methyl-1H-benzo[d]imidazol-2-yl)-V-cyclopentyl-piperidine-4-carboxamide;
1-(6-chloro-1-cyclobutyl-5-methyl-1H-benzo[d]imidazol-2-yl)-N-cyclopentylpiperidine-4-carboxamide;
N-cyclopentyl-1-(5,6-dichloro-1-cyclopentyl-1H-benzo[d]imidazol-2-yl)piperidine-4-carboxamide;
1-(1-(4-bromobenzyl)-5,6-dichloro-1H-benzo[d]imidazol-2-yl)-N-cyclopentylpiperidine-4-carboxamide;
N-cyclopentyl-1-(5,6-dichloro-1-((6-fluoropyridin-3-yl)methyl)-1H-benzo[d]imidazol-2-yl)piperidine-4-carboxamide;
N-cyclopentyl-1-(5,6-dichloro-4-(4-cyanobenzyl)-1H-benzo[d]imidazol-2-yl)piperidine-4-carboxamide;
N-cyclopentyl-1-(5,6-dichlor-1-(cyclopropylmethyl)-1H-benzo[d]imidazol-2-yl)piperidine-4-carboxamide;
N-cyclopentyl-1-(5,6-dichloro-1-((tetrahydrofuran-2-yl)methyl)-1H-benzo[d]imidazol-2-yl)piperidine-4-carboxamide;
N-cyclopentyl-1-(5,6-dichloro-1-((tetrahydro-2H-pyran-4-yl)methyl)-1H-benzo[d]imidazol-2-yl)piperidine-4-carboxamide;
N-cyclopentyl-1-(5,6-dichloro-1-(4-(methylcarbamoyl)benzyl)-1H-benzo[d]imidazol-2-yl)piperidine-4-carboxamide;
N-cyclopentyl-1-(5,6-dichloro-1-(4-(dimethylcarbamoyl)benzyl)-1H-benzo[d]imidazol-2-yl)piperidine-4-carboxamide;
1-(1-(4-carbamoylbenzyl)-5,6-dichloro-1H-benzo[d]imidazol-2-yl)-N-cyclopentylpiperidine+4-carboxamide;
N-cyclpentyl-1-(1-cyclopentyl-5,6-dimethyl-1H-benzo[d]imidazol-2-yl)piperidine-4-carboxamide;
1-(1-(4-chlorobenzyl)-5,6-dimethyl-1H-benzo[d]imidazol-2-yl)-N-cyclopentylpiperidine-4-carboxamide;
1-(1-(4-(1H-tetrazol-5-yl)benzyl)-5,6-dichloro-1H-benzo[d]imidazol-2-yl)-N-cyclopentylpiperidine-4-carboxamide;
N-cyclopentyl-5-(5,6-dichloro-1-(4-(methylsulfonylcarbamoyl)benzyl)-1H-benzo[d]imidazol-2-yl)piperidine-4-carboxamide;
N-butyl-1-(5,6-dichloro-1-cyclobutyl-1H-benzo[d]imidazol-2-yl)piperidine-4-carboxamide;
N-butyl-1-(5,6-dichloro-1-pentyl-1H-benzo[d]imidazol-2-yl)piperidine-4-carboxamide;
N-butyl-1-(5,6-dichloro-1-(4-fluorobenzyl)-1H-benzo[d]imidazol-2-yl)piperidine-4-carboxamide;
N-butyl-1-(5,6-dichroic-1-(6-fluoropyridin-3-yl)methyl)-1H-benzo[d]imidazol-2-yl)piperidine-4-carboxamide;
1-(5,6-dichloro-1-cyclobutyl-1H-benzo[d]imidazol-2-yl)-N-(tetrahydrofuran-3-yl)piperidine-4-carboxamide;
1-(5,6-dichloro-1-pentyl-1H-benzo[d]imidazol-2-yl)-N-(tetrahydrofuran-3-yl)piperidine-4-carboxamide;
1-(5,6-dichloro-1-(4-cyanobenzyl)-1N-benzo[d]imidazol-2-yl)-N-(tetrahydrofuran-3-yl)piperidine-4-carboxamide;
1-(5,6-dichloro-1-(cyclopropylmethyl)-1H-benzo[d]imidazol-2-yl)-N-(tetrahydrofuran-3-yl)piperidine-4-carboxamide;
1-(5,6-dichloro-1-((6-fluoropyridin-3-yl)methyl)-1H-benzo[d]imidazol-2-yl)-N-(tetrahydrofuran-3-yl)piperidine-4-carboxamide;
N-cyclopentyl-1-(5,6-dimethyl-1-((tetrahydro-2H-pyran-4-yl)methyl)-1H-benzo[d]imidazol-2-yl)piperidine-4-carboxamide;
1-(5,6-dichloro-1-cyclopentyl-1H-benzo[d]imidazol-2-yl)-N-(tetrahydrofuran-3-yl)piperidine-4-carboxamide;
1-(1-(4-bromobenzyl)-5,6-dichloro-1H-benzo[d]imidazol-2-yl)-N-(tetrahydrofuran-3-yl)piperidine-4-carboxamide;
N-cyclopentyl-1-(5,6-dimethyl-1-phenyl-1H-benzo[d]imidazol-2-yl)piperidine-4-carboxamide;
1-(1-(4-(H-tetrazol-5-yl)benzyl)-5,6-dimethyl-1H-benzo[d]imidazol-2-yl)-N-cyclopentylpiperidine-4-carboxamide,
1-(5,6-dichloro-1-(4-fluorobenzyl)-1H-benzo[d]imidazol-2-yl)-N-tetrahydrofuran-3-yl)piperidine-4-carboxamide;
1-(1-(4-(1H-1,2,4-triazol-1-yl)benzyl)-5,6-dichloro-1H-benzo[d]imidazol-2-yl)-N-cyclopentylpiperidine-4-carboxamide;
N-cyclopentyl-1-(5,6-dichloro-1-(2-methoxyethyl)-1H-benzo[d]imidazol-2-yl)piperidine-4-carboxamide;
N-cyclopentyl-1-(1-(4-fluorophenyl)-5,6-dimethyl-1H-benzo[d]imidazol-2-yl)piperidine-4-carboxamide;
1-(1-(cyclobutylmethyl)-5,6-dimethyl-1H-benzo[d]imidazol-2-yl)-N-cyclopentylpiperidine-4-carboxamide;
N-cyclopentyl-1-(5,6-dimethyl-1-(prop-2-ynyl)-H-benzo[d]imidazol-2-yl)piperidine-4-carboxamide;
N-cyclopentyl-1-(1-(2-ethylbutyl)-5,6-dimethyl-1H-benzo[d]imidazol-2-yl)piperidine-4-carboxamide;
N-cyclpentyl-1-(5,6-dimethyl-1-neopentyl-1H-benzo[d]imidazol-2-yl)piperidine-4-carboxamide;
1-(1-cyclohexyl-5,6-dimethyl-1H-benzo[d]imidazol-2-yl)-N-cyclopentylpiperidine-4-carboxamide;
1-(1-sec-butyl-5,6-dimethyl-1H-benzo[d]imidazol-2-yl)-N-cyclopentyl-piperidine-4-carboxamide;
N-cyclopentyl-1-(5,6-dichloro-1-(2-hydroxyethyl)-1H-benzo[d]imidazol-2-yl)piperidine-4-carboxamide;
N-cyclopentyl-1-(5,6-trimethyl-1H-benzo[d]imidazol-2-yl)piperidine-4-carboxamide;
N-cyclopentyl-1-(1-isobutyl-5,6-dimethyl-1H-benzo[d]imidazol-2-yl)piperidine-4-carboxamide;
1-(1-benzyl-5,6-dimethyl-1H-benzo[d]imidazol-2-yl)-N-cyclopentylpiperidine-4-carboxamide;
N-cyclopentyl-1-(1-(2-methoxyethyl)-5,6-dimethyl-1H-benzo[d]imidazol-2-yl)piperidine-4-carboxamide;
N-cyclopentyl-1-(1-(4-fluorobenzyl)-5,6-dimethyl-1H-benzo[d]imidazol-2-yl)piperidine-4-carboxamide;
1-(5-chloro-1-cyclobutyl-6-fluoro-1H-benzo[d]imidazol-2-yl)-N-cyclopentylpiperidine-4-carboxamide;
1-(6-chloro-1-cyclobutyl-5-fluor-1H-benzo[d]imidazol-2-yl)-N-cyclopentylpiperidine-4-carboxamide;
N-cyclopentyl-1-(5,6-dimethyl-1-(pentan-2-yl)-1H-benzo[d]imidazol-2-yl)piperidine-4-carboxamide;
N-butyl-1-(1-cyclobutyl-5,6-dimethyl-1H-benzo[d]imidazol-2-yl)piperidine-4-carboxamide;
N-butyl-1-(1-isopropyl-5,6-dimethyl-1H-benzo[d]imidazol-2-yl)piperidine-4-carboxamide;
N-butyl-1-(5,6-dimethyl-1-pentyl-1H-benzo[d]imidazol-2-yl)piperidine-4-carboxamide;
N-butyl-1-(1-4(6-fluoropyridin-3-yl)methyl)-5,6-dimethyl-1H-benzo[d]imidazol-2-yl)piperidine-4-carboxamide;
N-butyl-1-(1-(4-fluorobenzyl)-5,6-dimethyl-1H-benzo[d]imidazol-2-yl)piperidine-4-carboxamide;
N-benzyl-1-(1-isopropyl-5,6-dimethyl-1H-benzo[d]imidazol-2-yl)piperidine-4-carboxamide;
N-(4-fluorobenzyl)-1-(1-isopropyl-5,6-dimethyl-1H-benzo[d]imidazol-2-yl)piperidine-4-carboxamide;
N-(3-isopropoxypropyl)-1-(1-isopropyl-5,6-dimethyl-1H-benzo[d]imidazol-2-yl)piperidine-4-carboxamide;
N-(3,3-dimethylbutyl)-1-(1-isopropyl-5,6-dimethyl-1H-benzo[d]imidazol-2-yl)piperidine-4-carboxamide;
N-(4-bromobenzyl)-1-(1-isopropyl-5,6-dimethyl-1H-benzo[d]imidazol-2-yl)piperidine-4-carboxamide;
N-cyclohexyl-1-(1-isopropyl-5,6-dimethyl-1H-benzo[d]imidazol-2-yl)piperidine-4-carboxamide;
N-(4-cyanobenzyl)-1-(1-isopropyl-5,6-dimethyl-1H-benzo[d]imidazol-2-yl)piperidine-4-carboxamide;
(S)-1-(5,6-dimethyl-1-p-tolyl-1H-benzo[d]imidazol-2-yl)-N-(tetrahydrofuran-3-yl)piperidine-4-carboxamide;
4-((2-(4-(cyclopentylcarbamoyl)piperidin-1-yl)-5,6-dimethyl-1H-benzo[d]imidazol-1-yl)methyl)benzoic acid
1-(1-isopopyl-5,6-dimethyl-1H-benzo[d]imidazol-2-yl)-N-(thiazol-2-ylmethyl)piperidine-4-carboxamide;
1-(5,6-dichloro-1-isopopyl-1H-benzo[d]imidazol-2-yl)-N-(thiazol-2-ylmethyl)piperidine-4-carboxamide;
N-(4-(1H-tetrazol-5-yl)benzyl)-1-(1-isopropyl-5,6-dimethyl-1H-benzo[d]imidazol-2-yl)piperidine-4-carboxamide;
N-cyclopentyl-1-(5,6-dimethyl-1-(pentan-3-yl)-1H-benzo[d]imidazol-2-yl)piperidine-4-carboxamide;
1-(5,6-dimethyl-1-phenyl-1H-benzo[d]imidazol-2-yl)-N-(tetrahydrofuran-3-yl)piperidine-4-carboxamide;
N-cyclopentyl-1-(5,6-dichloro-1-(4-fluorophenyl)-1H-benzo[d]imidazol-2-yl)piperidine-4-carboxamide;
1-(1-(4-bromophenyl)-5,6-dichloro-1H-benzo[d]imidazol-2-yl)-N-(tetrahydrofuran-3-yl)piperidine-4-carboxamide;
1-(1-(4-fluorophenyl)-5,6-dimethyl-1H-benzo[d]imidazol-2-yl)-N-(tetrahydrofuran-3-yl)piperidine-4-carboxamide;
1-(1-(3-fluorophenyl)-5,6-dimethyl-1H-benzo[d]imidazol-2-yl)-N-(tetrahydrofuran-3-yl)piperidine-4-carboxamide;
1-(1-(3,5-difluorophenyl)-5,6-dimethyl-1H-benzo[d]imidazol-2-yl)-N-(tetrahydrofuran-3-yl)piperidine-4-carboxamide;
1-(1-(4-chlorophenyl)-5,6-dimethyl-1H-benzo[d]imidazol-2-yl)-N-(tetrahydrofuran-3-yl)piperidine-4-carboxamide;
1-(5,6-dimethyl-1-(4-(trifluoromethyl)phenyl)-1H-benzo[d]imidazol-2-yl)-N-(tetrahydrofuran-3-yl)piperidine-4-carboxamide;
1-(1-(3,5-dimethylphenyl)-5,6-dimethyl-1H-benzo[d]imidazol-2-yl)-N-(tetrahydrofuran-3-yl)piperidine-4-carboxamide;
1-(5,6-dimethyl-1-(naphthalen-2-yl)-1H-benzo[d]imidazol-2-yl)-N-(tetrahydrofuran-3-yl)piperidine-4-carboxamide;
1-(1-(benzo[d][1,3]dioxol-5-yl)-5,6-dimethyl-1H-benzo[d]imidazol-2-yl)-N-(tetrahydrofuran-3-yl)piperidine-4-carboxamide;
1-(5,6-dichloro-1-(3-methoxyphenyl)-1H-benzo[d]imidazol-2-yl)-N-(tetrahydrofuran-3-yl)piperidine-4-carboxamide;
1-(1-(benzo[d][1,3]dioxol-S-yl)-5,6-dichloro-1H-benzo[d]imidazol-2-yl)-N-(tetrahydrofuran-3-yl)pyridine-4-carboxamide;
1-(5,6-dichloro-1-(4-cyanophenyl)-1H-benzo[d]imidazol-2-yl)-N-(tetrahydrofuran-3-yl)piperidine-4-carboxamide;
1-cyclopentyl-1-(5,6-dimethyl-1-m-tolyl-1H-benzo[d]imidazol-2-yl)piperidine-4-carboxamide;
N-cyclpentyl-1-(1-(3,4-dimethylphenyl)-5,6-dimethyl-1H-benzo[d]imidazol-2-yl)piperidine-4-carboxamide;
N-cyclpentyl-1-(1-(4-fluoro-3-methylphenyl)-5,6-dimethyl-1H-benzo[d]imidazol-2-yl)piperidine-4-carboxamide;
N-cyclpentyl-1-(1-(4-ethylphenyl)-5,6-dimethyl-1H-benzo[d]imidazol-2-yl)piperidine-4-carboxamide;
N-cyclopentyl-1-(1-(4-isopropylphenyl)-5,6-dimethyl-1H-benzo[d]imidazol-2-yl)piperidine-4-carboxamide;
N-cyclopentyl-1-(5,6-dimethyl-1-(quinolin-6-yl)-1H-benzo[d]imidazol-2-yl)piperidine-4-carboxamide;
N-cyclopentyl-1-(5,6-dimethyl-1-(quinoxalin-6-yl)-1H-benzo[d]imidazol-2-yl)piperidine-4-carboxamide;
1-(5,6-dichloro-1-(4-(methylcarbamoyl)phenyl)-1H-benzo[d]imidazol-2-yl)-N-(tetrahydrofuran-3-yl)piperidine-4-carboxamide;
(R)-1-(5,6-dichloro-1-(3-methoxyphenyl)-1H-benzo[d]imidazol-2-yl)-N-(tetrahydrofuran-3-yl)piperidine-4-carboxamide;
N-cyclopentyl-1-(5,6-dichloro-1-(pyridin-3-yl)-1H-benzo[d]imidazol-2-yl)piperidine-4-carboxamide;
N-cyclopentyl-1-(5,6-dichlor-1-(pyridin-3-yl)-1H-benzo[d]imidazol-2-yl)piperadine-4-carboxamide;
N-cyclpentyl-1-(5,6-dichloro-1-(6-methoxypyridin-2-yl)-1H-benzo[d]imidazol-2-yl)piperidine-4-carboxamide;
N-cyclpentyl-1-(5,6-dichloro-1-(4-cyanophenyl)-H-benzo[d]imidazol-2-yl)piperidine-4-carboxamide;
(R)-1-(5,6-dichloro-1-(4-fluorophenyl)-H-benzo[d]imidazol-2-yl)-N-(tetrahydrofuran-3-yl)piperidine-4-carboxamide;
(R)-1-(5,6-dichloro-1-(3-fluorophenyl)-1H-benzo[d]imidazol-2-yl)-N-(tetrahydrofuran-3-yl)piperidine-4-carboxamide;
(R)-1-(5,6-dichloro-1-(4-methoxyphenyl)-1H-benzo[d]imidazol-2-yl)-N-(tetrahydrofuran-3-yl)piperidine-4-carboxamide;
(R)-1-(1-(4-tert-butylphenyl)-5,6-dichloro-1H-benzo[d]imidazol-2-yl)-N-tetrahydrofuran-3-yl)piperidine-4-carboxamide;
(R)-1-(5,6-dichloro-1-(4-(trifluoromethoxy)phenyl)-1H-benzo[d]imidazol-2-yl)-N-(tetrahydrofuran-3-yl)piperidine-4-carboxamide;
1-(5,6-dichloro-1-phenyl-1H-benzo[d]imidazol-2-yl)-N-(tetrahydrofuran-3-yl)piperidine-4-carboxamide;
1-(5,6-dichloro-1-(pyridin-2-yl)-1H-benzo[d]imidazol-2-yl)-N-(tetrahydrofuran-3-yl)piperidine-4-carboxamide;
1-(5,6-dichloro-1-(pyridin-3-yl)-1H-benzo[d]imidazol-2-yl)-N-(tetrahydrofuran-3-yl)piperidine-4-carboxamide;
1-(5,6-dichloro-1-p-tolyl-1H-benzo[d]imidazol-2-yl)-N-(tetrahydrofuran-3-yl)piperidine-4-carboxamide;
(R)-1-(1-(4-chloro-3-methoxyphenyl)-5,6-dimethyl-1H-benzo[d]imidazol-2-yl)-N-(tetrahydrofuran-3-yl)piperidine-4-carboxamide;
(R)-1-(5,6-dichloro-1-(4-chloro-3-methoxyphenyl)-1H-benzo[d]imidazol-2-yl)-N-(tetrahydrofuran-3-yl)piperidine-4-carboxamide;
(R)-1-(1-(2,3-dihydrobenzofuran-5-yl)-5,6-dimethyl-1H-benzo[d]imidazol-2-yl)-N-(tetrahydrofuran-3-yl)piperidine-4-carboxamide;
(R)-1-(5,6-dichloro-1-(2,3-dihydrobenzofuran-5-yl)-1H-benzo[d]imidazol-2-yl)-N-(tetrahydrofuran-3-yl)piperidine-4-carboxamide;
(R)-1-(1-(3,5-diethylphenyl)-5,6-dimethyl-1H-benzo[d]imidazol-2-yl)-N-(tetrahydrofuran-3-yl)piperidine-4-carboxamide;
1-(5,6-dichloro-1-(cyclobutylmethyl)-1H-benzo[d]imidazol-2-yl)-N-(tetrahydrofuran-3-yl)piperidine-4-carboxamide;
(R)-1-(1-(4-fluoro-3-methylphenyl)-5,6-dimethyl-1H-benzo[d]imidazol-2-yl)-N-(tetrahydrofuran-3-yl)piperidine-4-carboxamide;
(R)-1-(1-(4-ethylphenyl)-5,6-dimethyl-1H-benzo[d]imidazol-2-yl)-N-(tetrahydrofuran-3-yl)piperidine-4-carboxamide;
(R)-1-(1-(3,4-dimethylphenyl)-5,6-dimethyl-H-benzo[d]imidazol-2-yl)-N-(tetrahydrofuran-3-yl)piperidine-4-carboxamide;
(R)-1-(5,6-dimethyl-1-(quinolin-6-yl)-1H-benzo[d]imidazol-2-yl)-N-(tetrahydrofuran-3-yl)piperidine-4-carboxamide;
(R)-1-(1-(4-isopropylphenyl)-5,6-dimethyl-1H-benzo[d]imidazol-2-yl)-N-(tetrahydrofuran-3-yl)piperidine-4-carboxamide;
(R)-1-(1-(4-tert-butylphenyl)-5,6-dimethyl-1H-benzo[d]imidazol-2-yl)-N-(tetrahydrofuran-3-yl)piperidine-4-carboxamide;
(R)-1-(1-(3-chlorophenyl)-5,6-dimethyl-1H-benzo[d]imidazol-2-yl)-N-(tetrahydrofuran-3-yl)piperidine-4-carboxamide;
1-(6-chloro-5-methyl-1-phenyl-1H-benzo[d]imidazol-2-yl)-N-cyclopentylpiperidine-4-carboxamide;
(R)-1-(5-chloro-6-methyl-1-p-tolyl-1H-benzo[d]imidazol-2-yl)-N-(tetrahydrofuran-3-yl)piperidine-4-carboxamide;
(R)-1-(6-chloro-5-methyl-1-p-tolyl-1H-benzo[d]imidazol-2-yl)-N-(tetrahydrofuran-3-yl)piperidine-4-carboxamide;
(R)-1-(1-(4-carbamoylphenyl)-5-chloro-6-methyl-1H-benzo[d]imidazol-2-yl)-N-(tetrahydro furan-3-yl)piperidine-4-carboxamide;
1-(1-(4-carbamoylphenyl)-5-chloro-6-methyl-H-benzo[d]imidazol-2-yl)-cyclopentylpiperidine-4-carboxamide;
1-(4-carboxylphenyl)-6-chloro-1-methyl-1H-benzo[d]imidazol-2-yl)-N-cyclopentylpiperidine-4-carboxamide;
(R)-1-(1-(4-carbamoylphenyl)-6-chloro-5-methyl-1H-benzo[d]imidazol-2-yl)-N-(tetrahydrofuran-3-yl)piperidine-4-carboxamide;
(R)-1-(5-chloro-6-methyl-1-(4-trifluromethyl)phenyl)-1H-benzo[d]-imidazol-2-yl)-N-(tetrahydrofuran-3-yl)piperidine-4-carboxamide;
1-(5-chloro-6-methyl-1-(4-trifluormethyl)phenyl)-H-benzo[d]imidazol-2-yl)-N-cyclopentylpiperidine-4-carboxamide;
1-(5-chloro-6-methyl-1-(4-(trifluoromethyl)phenyl)-1H-benzo[d]imidazol-2-yl)-N-(tetrahydrofuran-3-yl)piperidine-4-carboxamide;
(R)-1-(6-chloro-5-methyl-1-(4-(trifluoromethyl)phenyl)-1H-benzo[d]-imidazol-2-yl)-N-(tetrahydrofuran-3-yl)piperidine-4-carboxamide;
1-(6-chloro-5-methyl-1-(4-(trifluoromethyl)phenyl)-1H-benzo[d]imidazol-2-yl)-N-(tetrahydrofuran-3-yl)piperidine-4-carbutamide;
1-(6-chloro-5-methyl-1-(4-trifluoromethyl)phenyl)-1H-benzo[d]imidazol-2-yl)-N-cyclopentylpiperidine-4-carboxamide;
(R)-1-(6-chloro-5-methyl-1-phenyl-1H-benzo[d]imidazol-2-yl)-N-(tetrahydrofuran-3-yl)piperidine-4-carboxamide;
(S)-1-(6-chloro-5-methyl-1-phenyl-1H-benzo[d]imidazol-2-yl)-N-(tetrahydrofuran-3-yl)piperidine-4-carboxamide;
N-butyl-1-(5-chloro-6-methyl-1-phenyl-1H-benzo[d]imidazol-2-yl)piperidine-4-carboxamide; N-butyl-1-(6-chloro-5-methyl-1-phenyl-1H-benzo[d]imidazol-2-yl)piperidine-4-carboxamide;
1-(5-chloro-6-methyl-1-phenyl-1H-benzo[d]imidazol-2-yl)-N-tetrahydrofuran-3-yl)piperidine-4-carboxamide;
1-(6-chloro-5-methyl-1-phenyl-1H-benzo[d]imidazol-2-yl)-N-(tetrahydrofuran-3-yl)piperidine-4-carboxamide;
1-(5-chloro-1-cyclobutyl-6-methyl-1H-benzo[d]imidazol-2-yl)-N-(tetrahydrofuran-3-yl)piperidine-4-carboxamide;
1-(6-chloro-1-cyclobutyl-5-methyl-1H-benzo[d]imidazol-2-yl)-N-(tetrahydrofuran-3-yl)piperidine-4-carboxamide;
1-(5-chloro-1-(cyclobutylmethyl)-6-methyl-1H-benzo[d]imidazol-2-yl)-N-cyclopentylpiperidine-4-carboxamide;
1-(6-chloro-1-(cyclobutylmethyl)-5-methyl-1H-benzo[d]imidazol-2-yl-N-cyclopentylpiperidine-4-carboxamide;
1-(5-chloro-6-methyl-1-pentyl-1H-benzo[d]imidazol-2-yl)-N-cyclopentylpiperidine-4-carboxamide;
1-(6-chloro-5-methyl-1-pentyl-1H-benzo[d]imidazol-2-yl)-N-cyclopentylpiperidine-4-carboxamide;
1-(5-chloro-1-(4-fluorophenyl)-6-methyl-1H-benzo[d]imidazol-2-yl)-N-cyclopentylpiperidine-4-carboxamide;
1-(6-chloro-1-(4-fluorophenyl)-5-methyl-1H-benzo[d]imidazol-2-yl)-N-cyclopentylpiperidine-4-carboxamide;
1-(5-chloro-6-methyl-1-phenyl-1H-benzo[d]imidazol-2-yl)-N-cyclopentylpiperidine-4-carboxamide;
1-(6-chloro-5-methyl-1-phenyl-1H-benzo[d]imidazol-2-yl)-N-cyclopentylpiperidine-4-carboxamide;
N-butyl-1-(5-chloro-1-cyclobutyl-6-methyl-1H-benzo[d]imidazol-2-yl)piperidine-4-carboxamide;
N-butyl-1-(6-chloro-1-cyclobutyl-5-methyl-1H-benzo[d]imidazol-2-yl)piperidine-4-carboxamide;
(R)-1-(5,6-dimethyl-1-(3-(trifluoromethyl)phenyl)-1H-benzo[d]imidazol-2-yl)-N-(tetrahydrofuran-3-yl)piperidine-4-carboxamide;
(R)-1-(1-(2,2-difluorobenzo[d][1,3]dioxol-5-yl)-5,6-dimethyl-1H-benzo[d]imidazol-2-yl)-N-(tetrahydrofuran-3-yl)piperidine-4-carboxamide;
(R)-1-(5,6-dimethyl-1-(5-(trifluoromethyl)pyridin-2-yl)-1H-benzo[d]imidazol-2-yl)-N-(tetrahydrofuran-3-yl)piperidine-4-carboxamide;
(R)-1-(1-(3-ethylphenyl)-5,6-dimethyl-1H-benzo[d]imidazol-2-yl)-N-(tetrahydrofuran-3-yl)piperidine-4-carboxamide;
(R)-1-(1-(3-isopropylphenyl)-5,6-dimethyl-1H-benzo[d]imidazol-2-yl)-N-(tetrahydrofuran-3-yl)piperidine-4-carboxamide;
(R)-1-(6-chloro-1-(4-isopropylphenyl)-5-methyl-1H-benzo[d]imidazol-2-yl)-N-(tetrahydrofuran-3-yl)piperidine-4-carboxamide;
(R)-1-(5,6-dichloro-1-(3-chlorophenyl)-1H-benzo[d]imidazol-2-yl)-N-(tetrahydrofuran-3-yl)piperidine-4-carboxamide;
(R)-1-(5,6-dichloro-4-(4-fluoro-3-methylphenyl)-1H-benzo[d]imidazol-2-yl)-N-(tetrahydrofuran-3-yl)piperidine-4-carboxamide;
(R)-1-(5,6-dichlor-1-(3,5-difluorophenyl)-H-benzo[d]imidazol-2-yl)-N-(tetrahydrofuran-3-yl)piperidine-4-carboxamide;
(R)-1-(5,6-dichloro-1-(3-cyanophenyl)-1H-benzo[d]imidazol-2-yl)-N-(tetrahydrofuran-3-yl)piperidine-4-carboxamide;
(R)-1-(5,6-dichloro-1-(pyridin-2-yl)-1H-benzo[d]imidazol-2-yl)-N-(tetrahydrofuran-3-yl)piperidine-4-carboxamide;
(R)-1-(5,6-dichloro-1-(5-methylpyridin-2-yl)-1H-benzo[d]imidazol-2-yl)-N-(tetrahydrofuran-3-yl)piperidine-4-carboxamide;
(R)-1-(5,6-dichloro-1-(6-methoxypyridin-2-yl)-1H-benzo[d]imidazol-2-yl)-N-tetrahydrofuran-3-yl)piperidine-4-carboxamide;
(R)-1-(5,6-dichloro-1-(3-isopropylphenyl)-1H-benzo[d]imidazol-2-yl)-N-(tetrahydrofuran-3-yl)piperidine-4-carboxamide;
(R)-1-(5,6-dichloro-1-(4-methylpyridin-2-yl)-H-benzo[d]imidazol-2-yl)-N-(tetrahydrofuran-3-yl)piperidine-4-carboxamide;
(R)-1-(5,6-dichloro-1-(6-methylpyridin-2-yl)-1H-benzo[d]imidazol-2-yl)-N-(tetrahydrofuran-3-yl)piperidine-4-carboxamide;
(R)-1-(5,6-dichloro-1-(3-(difluoromethoxy)phenyl)-1H-benzo[d]imidazol-2-yl)-N-(tetrahydrofuran-3-yl)piperidine-4-carboxamide;
(R)-1-(5,6-dichloro-1-(3-(trifluoromethoxy)phenyl)-1H-benzo[d]imidazol-2-yl)-N-(tetrahydrofuran-3-yl)piperidine-4-carboxamide;
(R)-1-(5,6-dichloro-1-(4-isobutylphenyl)-1H-benzo[d]imidazol-2-yl)-N-(tetrahydrofuran-3-yl)piperidine-4-carboxamide;
(R)-1-(5,6-dichloro-1-(3,4-dimethylphenyl)-1H-benzo[d]imidazol-2-yl)-N-(tetrahydrofuran-3-yl)piperidine-4-carboxamide;
(R)-1-(5,6-dichloro-1-(4-chlorphenyl)-1H-benzo[d]imidazol-2-yl)-N-(tetrahydrofuran-3-yl)piperidine-4-carboxamide;
(R)-1-(5,6-dichloro-1-(5-(trifluoromethyl)pyridin-2-yl)-1H-benzo[d]imidazol-2-yl)-N-(tetrahydrofuran-3-yl)piperidine-4-carboxamide;
(R)-1-(5,6-dichloro-1-(5-fluoropyridin-2-yl)-1H-benzo[d]imidazol-2-yl)-N-(tetrahydrofuran-3-yl)piperidine-4-carboxamide;
(R)-1-(1-(4-isobutylphenyl)-5,6-dimethyl-H-benzo[d]imidazol-2-yl)-N-(tetrahydrofuran-3-yl)piperidine-4-carboxamide;
(R)-1-(6-chloro-1-(3,5-dimethylphenyl)-5-methyl-1H-benzo[d]imidazol-2-yl)-N-(tetrahydrofuran-3-yl)piperidine-4-carboxamide;
(R)-1-(1-(4-chloro-3-fluorophenyl)-5,6-dimethyl-1H-benzo[d]imidazol-2-yl)-N-(tetrahydrofuran-3-yl)piperidine-4-carboxamide
(R)-1-(5,6-dichloro-1-(4-chloro-3-fluorophenyl)-1H-benzo[d]imidazol-2-yl(tetrahydrofuran-3-yl)piperidine-4-carboxamide;
(R)-1-(5,6-dichlor-1-(3,5-diethylphenyl)-H-benzo[d]imidazol-2-yl)-N-(tetrahydrofuran-3-yl)piperidine-4-carboxamide;
(R)-1-(1-(3-tert-butylphenyl)-5,6-dimethyl-1H-benzo[d]imidazol-2-yl)-N-(tetrahydrofuran-3-yl)piperidine-4-carboxamide;
(R)-1-(1-(3-tert-butylphenyl)-5,6-dichloro-1H-benzo[d]imidazol-2-yl)-N-(tetrahydrofuran-3-yl)piperidine-4-carboxamide;
(R)-1-(5,6-dichloro-1-phenyl-1H-benzo[d]imidazol-2-yl)-N-(tetrahydrofuran-3-yl)piperidine-4-carboxamide;
(R)-1-(5,6-dichloro-1-(4-ethylphenyl)-1H-benzo[d]imidazol-2-yl)-N-(tetrahydrofuran-3-yl)piperidine-4-carboxamide;
(R)-1-(5,6-dichloro-1-m-tolyl-1H-benzo[d]imidazol-2-yl)-N-(tetrahydrofuran-3-yl)piperidine-4-carboxamide;
(R)-1-(5,6-dichloro-1-(3,5-dimethylphenyl)-H-benzo[d]imidazol-2-yl)-N-(tetrahydrofuran-3-yl)piperidine-4-carboxamide;
(R)-1-(5,6-dichloro-1-(3-ethylphenyl)-1H-benzo[d]imidazol-2-yl)-N-(tetrahydrofuran-3-yl)piperidine-4-carboxamide;
(R)-1-(5,6-dichloro-1-(6-(trifluoromethyl)pyridin-2-yl)-1 I-benzo[d]imidazol-2-yl)-N-(tetrahydrofuran-3-yl)piperidine-4-carboxamide;
(R)-1-(5,6-dichloro-1-(3-ethoxyphenyl)-1H-benzo[d]imidazol-2-yl)-N-(tetrahydrofuran-3-yl)piperidine-4-carboxamide;
(R)-1-(5,6-dichloro-1-(3-isopropoxyphenyl)-JH-benzo[d]imidazol-2-yl)-N-(tetrahydrofuran-3-yl)piperidine-4-carboxamide;
(R)-1-(5,6-dichloro-1-(3-propoxyphenyl)-1H-benzo[d]imidazol-2-yl)-N-(tetrahydrofuran-3-yl)piperidine-4-carboxamide;
(R)-1-(1-(4-fluoro-3,5-dimethylphenyl)-5,6-dimethyl-1H-benzo[d]imidazol-2-yl)-N-(tetrahydrofuran-3-yl)piperidine-4-carboxamide;
(R)-1-(5,6-dichloro-1-(4-chloro-3-methylphenyl)-1H-benzo[d]imidazol-2-yl)-N-(tetrahydrofuran-3-yl)piperidine-4-carboxamide;
(R)-1-(5,6-dichloro-1-(3-chloro-4-methylphenyl)-1H-benzo[d]imidazol-2-yl)-N-(tetrahydrofuran-3-yl)piperidine-4-carboxamide;
(R)-1-(5,6-dimethyl-1-phenyl-1H-benzo[d]imidazol-2-yl)-N-(tetrahydrofuran-3-yl)piperidine-4-carboxamide;
(R)-1-(5,6-dimethyl-1-p-tolyl-1H-benzo[d]imidazol-2-yl)-N-(tetrahydrofuran-3-yl)piperidine-4-carboxamide;
(R)-1-(5,6-dimethyl-1-m-tolyl-1H-benzo[d]imidazol-2-yl)-N-(tetrahydrofuran-3-yl)piperidine-4-carboxamide;
(R)-1-(1-(3-cyanophenyl)-5,6-dimethyl-1H-benzo[d]imidazol-2-yl)-N-(tetrahydrofuran-3-yl)piperidine-4-carboxamide;
(R)-1-(5,6-dimethyl-1-(naphthalen-2-yl)-1H-benzo[d]imidazol-2-yl)-N-(tetrahydrofuran-3-yl)piperidine-4-carboxamide;
(R)-1-(1-(4-chloro-3-methylphenyl)-5,6-dimethyl-1H-benzo[d]imidazol-2-yl)-N-(tetrahydrofuran-3-yl)piperidine-4-carboxamide;
(R)-1-(1-(3-chloro-4-methylphenyl)-5,6-dimethyl-1H-benzo[d]imidazol-2-yl)-N-(tetrahydrofuran-3-yl)piperidine-4-carboxamide;
(R)-1-(1-(benzo[d]thiazol-6-yl)-5,6-dimethyl-1H-benzo[d]imidazol-2-yl)-N-(tetrahydrofuran-3-yl)piperidine-4-carboxamide;
(R)-1-(1-(benzo[d]thiazol-5-yl)-5,6-dimethyl-1H-benzo[d]imidazol-2-yl)-N-(tetrahydrofuran-3-yl)piperidine-4-carboxamide;
(R)-1-(5-chloro-1-(3,4-dimethylphenyl)-6-methyl-1H-benzo[d]imidazol-2-yl)-N-(tetrahydrofuran-3-yl)piperidine-4-carboxamide;
(R)-1-(6-chloro-1-(3,4-dimethylphenyl)-5-methyl-1H-benzo[d]imidazol-2-yl)-N-(tetrahydrofuran-3-yl)piperidine-4-carboxamide;
(R)-1-(5-chloro-1-(4-isopropylphenyl)-6-methyl-1H-benzo[d]imidazol-2-yl)-N-(tetrahydrofuran-3-yl)piperidine-4-carboxamide;
(R)-1-(6-chloro-1-(4-isopropylphenyl)-5-methyl-1H-benzo[d]imidazol-2-yl)-N-(tetrahydrofuran-3-yl)piperidine-1-carboxamide;
(S)-1-(5-chloro-6-methyl-1-p-tolyl-1H-benzo[d]imidazol-2-yl)-N-(tetrahydrofuran-3-yl)piperidine-4-carboxamide;
(S)-1-(6-chloro-5-methyl-1-p-tolyl-1H-benzo[d]imidazol-2-yl)-N-(tetrahydrofuran-3-yl)piperidine-4-carboxamide;
(S)-1-(6-chloro-1-(3,5-dimethylphenyl)-5-methyl-1H-benzo[d]imidazol-2-yl)-N-(tetrahydrofuran-3-yl)piperidin-4-carboxamide;
(R)-1-(5,6-dichloro-1-p-tolyl-1H-benzo[d]imidazol-2-yl)-N-(tetrahydrofuran-3-yl)piperidine-4-carboxamide;
1-(1-cyclobutyl-5,6-dimethyl-1H-benzo[d]imidazol-2-yl)-N-((1S,2S)-2-hydroxycyclopentyl)piperidine-4-carboxamide;
1-(1-cyclobutyl-5,6-dimethyl-1H-benzo[d]imidazol-2-yl)-N-((1R,2R)-2-hydroxycyclopentyl)piperidine-4-carboxamide;
(R)-1-(1-(benzofuran-5-yl)-5,6-dimethyl-1H-benzo[d]imidazol-2-yl)-N-4-tetrahydrofuran-3-yl)piperidine-4-carboxamide;
(R)-1-(5,6-dichloro-1-(4-fluoro-3,5-dimethylphenyl)-1H-benzo[d]imidazol-2-yl)-N-(tetrahydrofuran-3-yl)piperidine-4-carboxamide;
(R)-1-(1-(1-(3-fluoro-5-methylphenyl)-5,6-dimethyl-1H-benzo[d]imidazol-2-yl)-N-(tetrahydrofuran-3-yl)piperidine-4-carboxamide;
(R)-1-(5,6-dichloro-1-(3-fluoro-5-methylphenyl)-1H-benzo[d]imidazol-2-yl)-N-(tetrahydrofuran-3-yl)piperidine-4-carboxamide;
(R)-1-(5,6-dichloro-1-(4-cyclopropylphenyl)-H-benzo[d]imidazol-2-yl)-N-(tetrahydropurin-3-yl)piperidine-4-carboxamide;
(R)-1-(5,6-dichloro-1-(3-cyclopropylphenyl)-1H-benzo[d]imidazol-2-yl)-N-(tetrahydrofuran-3-yl)piperidine-4-carboxamide;
(R)-1-(1-(benzofuran-6-yl)-5,6-dimethyl-1H-benzo[d]imidazol-2-yl)-N-(tetrahydrofuran-3-yl)piperidine-4-carboxamide;
(R)-1-(5-chloro-1-(4-ethylphenyl)-6-methyl-1H-benzo[d]imidazol-2-yl)-N-(tetrahydrofuran-3-yl)piperidin-4-carboxamide;
(R)-1-(6-chloro-(4-ethylphenyl)-5-methyl-1H-benzo[d]imidazol-2-yl)-N-(tetrahydrofuran-3-yl)piperidine-4-carboxamide;
(R)-1-(5-chloro-1-(4-ethylphenyl)-6-methyl-1H-benzo[d]imidazol-2-yl)-N-(tetrahydrofuran-3-yl)piperidine-4-carboxamide;
(R)-1-(1-(4-tert-butylphenyl)-6-chloro-5-methyl-1H-benzo[d]imidazol-2-yl)-N-(tetrahydrofuran-3-yl)piperidine-4-carboxamide;
(R)-1-(1-(4-cyclopropylphenyl)-5,6-dimethyl-1-benzo[d]imidazol-2-yl)-N-(tetrahydrofuran-3-yl)piperidine-4-carboxamide;
(R)-1-(1-(3-cyclopropylphenyl)-5,6-dimethyl-1H-benzo[d]imidazol-2-yl)-N-(tetrahydrofuran-3-yl)piperidine-4-carboxamide;
(R)-1-(5,6-dichloro-1-(3-(cyclopentyloxy)phenyl)-1H-benzo[d]imidazol-2-yl)-N-(tetrahydrofuran-3-yl)piperidine-4-carboxamide;
(R)-1-(5,6-dichloro-1-(3-cyclobutoxyphenyl)-1H-benzo[d]imidazol-2-yl)-N-(tetrahydrofuran-3-yl)piperidine-4-carboxamide;
1-(5-chloro-6-fluoro-1-methyl-1H-benzo[d]imidazol-2-yl)-N-cyclopentylpiperidine-4-carboxamide;
1-(6-chloro-5-fluoro-1-methyl-1H-benzo[d]imidazol-2-yl)-N-cyclopentylpiperidine-4-carboxamide;
1-(5-chloro-1-ethyl-6-fluoro-1H-benzo[d]imidazol-2-yl)-N-cyclopentylpiperidine-4-carboxamide;
1-(6-chloro-1-ethyl-5-fluoro-H-benzo[d]imidazol-2-yl)-N-cyclopentylpiperidine-4-carboxamide
1-(5-chloro-6-fluoro-1-isopropyl-1H-benzo[d]imidazol-2-yl)-N-cyclopentylpiperidine-4-carboxamide;
1-(6-chloro-5-fluoro-1-isopropyl-1H-benzo[d]imidazol-2-yl)-N-cyclopentylpiperidine-4-carboxamide;
1-(1-butyl-5-chloro-6-fluoro-1H-benzo[d]imidazol-2-yl)-N-cyclopentylpiperidine-4-carboxamide;
1-(1-butyl-6-choro-5-fluoro-1H-benzo[d]imidazol-2-yl)-N-cyclopentylpiperidine-4-carboxamide,
1-(5,6-dimethyl-1-p-tolyl-1H-benzo[d]imidazol-2-yl)-N-(tetrahydrofuran-3-yl)piperidine-4-carboxamide;
(R)-1-(6-chloro-1-(4-fluorophenyl)-5-methyl-1H-benzo[d]imidazol-2-yl)-N-(tetrahydrofuran-3-yl)piperidine-4-carboxamide;
(R)-1-(5-chloro-1-(4-fluorophenyl)-6-methyl-1H-benzo[d]imidazol-2-yl)-N-(tetrahydrofuran-3-yl)piperidine-4-carboxamide;
(R)-1-(6-choro-5-methyl-1-m-tolyl-1H-benzo[d]imidazol-2-yl)-N-(tetrahydrofuran-3-yl)piperidine-4-carboxamide;
(R)-1-(5-chloro-6-methyl-1-m-tolyl-H-benzo[d]imidazol-2-yl)-N-(tetrahydrofuran-3-yl)piperidine-4-carboxamide;
(R)-1-(5-chloro-6-methyl-1-(5-(trifluormethyl)pyridin-2-yl)-1H-benzo[d]imidazol-2-yl)-N-(tetrahydrofuran-3-yl)piperidine-4-carboxamide;
(R)-1-(6-chloro-5-methyl-1-(5-(trifluoromethyl)pyridin-2-yl)-1H-benzo[d]imidazol-2-yl)-N-(tetrahydrofuran-3-yl)piperidine-4-carboxamide;
1-(1-cyclobutyl-5,6-dimethyl-1H-benzo[d]imidazol-2-yl)-N-((2S)-2-methoxycyclopentyl)piperidine-4-carboxamide;
1-(1-cyclobutyl-5,6-dimethyl-1H-benzo[d]imidazol-2-yl)-N-((2S)-2-ethoxycyclopentyl)piperidine-4-carboxamide;
1-(1-cyclobutyl-5,6-dimethyl-1H-benzo[d]imidazol-2-yl)-N-((2S)-2-propoxycyclopentyl)piperidine-4-carboxamide;
1-(5,6-dichlor-1-(4-(dimethylcarbamoyl)phenyl)-1H-benzo[d]imidazol-2-yl)-N-(tetrahydrofuran-3-yl)piperidine-4-carboxamide;
1-(5,6-dichloro-1-cyclobutyl-1H-benzo[d]imidazol-2-yl)-N-((1S,2S)-2-hydroxycyclopentyl)piperidine-4-carboxamide;
1-(5,6-dichloro-1-cyclobutyl-1H-benzo[d]imidazol-2-yl)-N-((1S,2S)-2-methoxycyclopentyl)piperidine-4-carboxamide;
1-(5,6-dichloro-1-cyclobutyl-1H-benzo[d]imidazol-2-yl)-N-((1S,2S)-2-ethoxycyclopentyl)piperidine-4-carboxamide;
1-(1-cyclobutyl-5,6-dimethyl-H-benzo[d]imidazol-2-yl)-N-(furan-2-ylmethyl)piperidine-4-carboxamide;
1-(1-cyclobutyl-5,6-dimethyl-1H-benzo[d]imidazol-2-yl)-N-(thiophen-2-ylmethyl)piperidine-4-carboxamide;
(R)-1-(5-bromopyridin-2-yl)-5,6-dimethyl-1H-benzo[d]imidazol-2-yl)-N-(tetrahydrofuran-3-yl)piperidine-4-carboxamide;
(R)-1-(1-(5-ethylpyridin-2-yl)-5,6-dimethyl-1H-benzo[d]imidazol-2-yl)-N-(tetrahydrofuran-3-yl)piperidine-4-carboxamide;
(R)-1-(1-(3-chloro-5-fluorophenyl)-5,6-dimethyl-1H-benzo[d]imidazol-2-yl)-N-(tetrahydrofuran-3-yl)piperidine-4-carboxamide;
(R)-1-(5,6-dichloro-1-(3-chloro-5-fluorophenyl)-1H-benzo[d]imidazol-2-yl)-N-(tetrahydrofuran-3-yl)piperidine-4-carboxamide;
(R)-1-(5,6-dichloro-1-(3-(trifluoromethyl)phenyl)-H-benzo[d]imidazol-2-yl)-N-(tetrahydrofuran-3-yl)piperidine-4-carboxamide;
(R)-1-(5,6-dichloro-1-(2,2-difluorobenzo[d][1,3]dioxol-5-yl)-H-benzo[d]imidazol-2-yl)-N-(tetrahydrofuran-3-yl)piperidine-4-carboxamide;
(S)-1-(1-(3,5-dimethylphenyl)-5,6-dimethyl-1H-benzo[d]imidazol-2-yl)-N-(tetrahydrofuran-3-yl)piperidine-4-carboxamide;
(R)-1-(5,6-dichloro-1-(4-(trifluoromethyl)pyridin-2-yl)-1H-benzo[d]imidazol-2-yl)-N-(tetrahydrofuran-3-yl)piperidine-4-carboxamide;
(R)-1-(5,6-dichloro-1-(5-chloropyridin-2-yl)-H-benzo[d]imidazol-2-yl)-N-(tetrahydrofuran-3-yl)piperidine-4-carboxamide;
(R)-1-(5,6-dichloro-1-(5-fluoro-6-methylpyridin-2-yl)-1H-benzo[d]imidazol-2-yl)-N-(tetrahydrofuran-3-yl)piperidine-4-carboxamide;
(R)-1-(1-(3-fluorophenyl)-5,6-dimethyl-1H-benzo[d]imidazol-2-yl)-N-(tetrahydrofuran-3-yl)piperidine-4-carboxamide;
(R)-1-(1-(3,5-dimethylphenyl)-5,6-dimethyl-1H-benzo[d]imidazol-2-yl)-N-(tetrahydrofuran-3-yl)piperidine-4-carboxamide;
(R)-1-(1-(4-fluorophenyl)-5,6-dimethyl-1H-benzo[d]imidazol-2-yl)-N-(tetrahydrofuran-3-yl)piperidine-4-carboxamide;
(R)-1-(1-(3,5-difluo phenyl)-5,6-dimethyl-H-benzo[d]imidazol-2-yl)-N-(tetrahydrofuran-3-yl)piperidine-4-carboxamide;
(R)-1-(1-(4-chlorophenyl)-5,6-dimethyl-1H-benzo[d]imidazol-2-yl)-N-(tetrahydrofuran-3-yl)piperidine-4-carboxamide;
(R)-1-(5,6-dimethyl-1-(4-(trifluoromethyl)phenyl)-1H-benzo[d]imidazol-2-yl)-N-(tetrahydrofuran-3-yl)piperidine-4-carboxamide;
(R)-1-(5,6-dimethyl-1-p-tolyl-1H-benzo[d]imidazol-2-yl)-N-(tetrahydrofuran-3-yl)piperidine-4-carboxamide;
(R)-1-(1-(5-chloropyridin-2-yl)-5,6-dimethyl-1H-benzo[d]imidazol-2-yl)-N-(tetrahydrofuran-3-yl)piperidine-4-carboxamide;
(R)-1-(5,6-dimethyl-1-(4-(trifluoromethyl)pyridin-2-yl)-1H-benzo[d]imidazol-2-yl)-N-(tetrahydrofuran-3-yl)piperidine-4-carboxamide;
(R)-1-(1-(5-fluoro-6-methylpyridin-2-yl)-5,6-dimethyl-1H-benzo[d]imidazol-2-yl)-N-(tetrahydrofuran-3-yl)piperidine-4-carboxamide;
(R)-1-(5,6-dichloro-1-(4-ethoxyphenyl)-1H-benzo[d]imidazol-2-yl)-N-(tetrahydrofuran-3-yl)piperidine-4-carboxamide;
(R)-1-(5,6-dichloro-1-(4-methoxy-3-methylphenyl)-1H-benzo[d]imidazol-2-yl)-N-(tetrahydrofuran-3-yl)piperidine-4-carboxamide; and
(R)-1-(1-cyclobutyl-5,6-dimethyl-1H-benzo[d]imidazol-2-yl)-N-(tetrahydrofuran-3-yl)piperidine-4-carboxamide;

or a pharmaceutically acceptable salt thereof.

In particular embodiments, compounds that may be utilised as the required mPGES-1 Inhibitor may include the following compounds forming Group E below, or a pharmaceutically acceptable salt thereof:

LY-3023703 (Eli Lilly and Company development compound);

AZ-13330908 (AstraZeneca development compound); GRC-27864 (Glenmark Pharmaceuticals development compound, as depicted below)

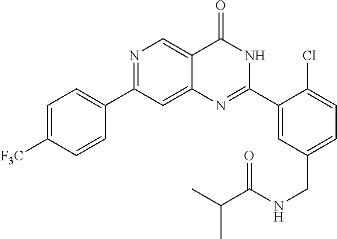 [SMILES: CC(C)C(=O)NCc1ccc(Cl)c(-c2nc3cc(-c4ccc(C(F)(F)F)cc4)ncc3c(=O)[nH]2)c1]

As described herein, the present invention relates to the use of compounds that are mPGES-1 inhibitors in the treatment of diseases and disorders characterised by vasoconstriction.

The skilled person will understand that references to diseases and disorders characterised by vasoconstriction will include references to diseases and disorders that have vasoconstriction as a significant component (either as a causative factor in the development of the condition or as a symptom of other causative factors). Such diseases and disorders will be readily identified by those skilled in the art.

In particular, the skilled person will understand that diseases and disorders characterised by vasoconstriction may result from (i.e. be a symptom of) the effect of an underlying causative factor, which may itself be an underlying disease or disorder, such as inflammation. Alternatively, such diseases and disorders may be idiopathic.

In a particular embodiment, the disease or disorder characterised by vasoconstriction may be associated with (e.g. result from) inflammation.

In alternative embodiments, the disease or disorder may be described as being inflammation characterised by (i.e. associated with, e.g. resulting from) vasoconstriction.

Particular diseases and disorders that may result in (and thus be characterised by) vasoconstriction (in particular, vasoconstriction associated with inflammation) include autoimmune disorders, such as scleroderma.

Thus, in a particular embodiment, the disease or disorder (i.e. the disease or disorder that may be characterised by vasoconstriction) is scleroderma (e.g. which may be referred to as vasoconstriction associated with scleroderma).

In particular, the treatment or prophylaxis using a compound that is an mPGES-1 inhibitor as defined herein may be referred to as the treatment or prophylaxis of scleroderma, such as vasoconstriction resulting from (i.e. caused by or associated with) scleroderma.

Alternatively, the treatment or prophylaxis using a compound that is an mPGES-1 inhibitor as defined herein may be referred to as the treatment or prophylaxis of inflammation associated with (e.g. resulting from) scleroderma, such as vasoconstriction resulting from (i.e. caused by or associated with) inflammation associated with (e.g. resulting from) scleroderma.

In cases where the vasoconstriction is an effect of an underlying disease or disorder, references to treatment of the disease or disorder may refer in particular to treatment of the vasoconstriction per se (although other factors relating to the disease or disorder may also be treated concomitantly, such as associated inflammation). In such instances, references to the treatment of vasoconstriction may indicate that the level of constriction (i.e. the narrowing) of the blood vessels is reduced (e.g. by a measurable and therapeutically significant amount) such that the width of blood vessel is increased when compared to the untreated (i.e. constricted) state.

Thus, in particular embodiments, the treatment (i.e. the treatment of the disease or disorder characterised by vasoconstriction) comprises providing a reduction in the degree of vasoconstriction.

Further, in cases where the vasoconstriction is an effect of an underlying disease or disorder, references to treatment of the disease or disorder may refer in particular to treatment of the particular disease or disorder (e.g. by treatment of associated inflammation) in a manner that results in a lower degree of vasoconstriction than would be observed when such conditions are treated using alternative methods (e.g. in the case of the treatment of associated inflammation, a reduction in vasoconstriction compared to the degree of vasoconstriction observed when such conditions are treated using COX inhibitors, such as COX-2 inhibitors, as known to those skilled in the art).

Thus, in particular embodiments, the treatment (i.e. the treatment of the disease or disorder characterised by vasoconstriction) comprises treatment of inflammation (e.g. the treatment of inflammation associated with the relevant disease or disorder) with a lower degree of vasoconstriction (such as at least a 10% lower degree, e.g. at least a 20%, 30%, 40% or 50% lower degree) than that observed in treatment with a COX-2 inhibitor.

In more particular embodiments, the treatment (i.e. the treatment of the disease or disorder characterised by vasoconstriction) comprises treatment of inflammation (i.e. the treatment of inflammation associated with the relevant disease or disorder) such that there is the substantial absence of an increase in vasoconstriction (e.g. with a measurable degree of constriction of the relevant blood vessels that is not greater than 20% of their normal width, such as a degree of constriction of blood vessels that is not greater than 10% (e.g. not greater than 5%) of their normal width).

The skilled person will also understand that inflammatory conditions such as scleroderma may also lead to secondary diseases and disorders, the symptoms of which may be treated through treatment of the underlying condition.

Thus, in particular embodiments, the treatment or prophylaxis as described herein may be treatment or prophylaxis of a disease or disorder associated with scleroderma.

Particular diseases and disorders characterised by vasoconstriction (for example, vasoconstriction associated with inflammation, such as vasoconstriction associated with scleroderma) that may be mentioned include:

cardiovascular disorders, such as Raynaud's phenomenon (also known as secondary Raynaud's, such as wherein systemic lupus erythematosus (SLE) or, particularly, scleroderma is the primary disease), Raynaud's disease (also known as primary Raynaud's), healed pitting ulcers on the fingertips, digital ulcers, skin and mucousal telangiectasis, palpitations, irregular heart rate and fainting due to conduction abnormalities and congestive heart failure;

digestive disorders, such as gastroesophageal reflux disease (GERD/GORD), bloating, indigestion, loss of appetite, diarrhoea alternating with constipation, sicca syndrome and its complications, loosening of teeth and hoarseness (due to acid reflux);

pulmonary disorders, such as pulmonary artery hypertension (PAH), progressive worsening of shortness of breath, chest pain (particularly when due to pulmonary artery hypertension) and dry, persistent cough (e.g. due to interstitial lung disease);

musculoskeletal disorders, such as joint, muscle aches, loss of joint range of motion, carpal tunnel syndrome and muscle weakness;

genitourinary disorders, such as erectile dysfunction, dyspareunia, scleroderma renal crises and kidney failure; and

other disorders such as facial pain due to trigeminal neuralgia, hand paraesthesias, headache, stroke, fatigue, calcinosis and weight loss.

Particular diseases and disorders characterised by vasoconstriction that may be mentioned include Raynaud's phenomenon (e.g. secondary Raynaud's associated with (e.g. resulting from) SLE or, particularly, scleroderma), conditions associated with Raynaud's phenomenon (such as digital ulcers), and pulmonary arterial hypertension (PAH).

Thus, in a particular embodiment, the disease or disorder is Raynaud's phenomenon (e.g. Raynaud's phenomenon associated with (e.g. resulting from) an inflammatory condition, such as SLE or, particularly, scleroderma).

In a more particular embodiment, the disease or disorder is Raynaud's phenomenon associated with scleroderma.

In an alternative embodiment, the treatment or prophylaxis is of inflammation associated with (or resulting in) Raynaud's phenomenon, such as that resulting from scleroderma.

In a particular embodiment, the disease or disorder is digital ulcers, such as digital ulcers associated with scleroderma and/or Raynaud's phenomenon.

In a further embodiment, the disease or disorder is pulmonary arterial hypertension (PAH), such as PAH associated with (e.g. resulting from) an inflammatory condition, such as SLE or, particularly, scleroderma.

In an alternative embodiment, the treatment or prophylaxis is of inflammation associated with (or resulting in) PAH, such as that resulting from scleroderma.

For the avoidance of doubt, the disease or disorder may be pulmonary arterial hypertension (PAH) that is not associated with scleroderma, such as idiopathic PAH.

For the avoidance of doubt, in particular embodiments there is provided the treatment or prophylaxis of a disease or disorder selected from Raynaud's phenomenon, such as that resulting from scleroderma, and pulmonary arterial hypertension (PAH).

For the avoidance of doubt, in particular embodiments the disease or disorder may be Raynaud's phenomenon or pulmonary arterial hypertension, each associated with scleroderma and/or SLE.

In further embodiments, the vasoconstriction may be associated with (e.g. be a symptom of) vascular complications associated with diabetes mellitus. In particular, the vasoconstriction may result from vascular dysfunction due to increased oxidative stress, impaired NO formation, dyslipidemia and inflammation, which all together cause vasoconstriction (thus reducing blood circulation).

As used herein, references to diabetes mellitus will include references to type 2 and type 2 diabetes. In particular, they will refer to type 1 diabetes.

Thus, in a particular embodiment, there is provided the treatment or prophylaxis of vasoconstriction associated with (e.g. resulting from) diabetes mellitus (e.g. type 1 diabetes).

In particular, vasoconstriction associated with (e.g. resulting from) diabetes mellitus (e.g. type 1 diabetes) may lead to conditions such as poor wound healing, diabetic neuropathy (e.g. peripheral diabetic neuropathy), diabetic retinopathy and diabetic ulcers. Moreover, vasoconstriction associated with (e.g. resulting from) diabetes mellitus (e.g. type 1 diabetes) may increase the risk of stroke (i.e. thrombotic stroke), pulmonary embolism and myocardial infarction, the treatment and, in particular, prophylaxis of which may be achieved as part of the present invention.

In a more particular embodiment, there is provided the treatment or prophylaxis of diabetic neuropathy (e.g. peripheral diabetic neuropathy), diabetic retinopathy or diabetic ulcers.

In a yet more particular embodiment, there is provided the treatment or prophylaxis of diabetic ulcers.

The skilled person will appreciate that the treatment or prophylaxis of a disease or disorder characterised by vasoconstriction may be achieved in combination with the treatment or prophylaxis of a further (associated or underlying) disease or disorder, such as inflammation. In other words, the disease or disorder characterised by vasoconstriction may also have an inflammatory component, the treatment or prophylaxis of which may also be achieved as part of the present invention.

As described herein, the present invention may be particularly suited to the treatment or prophylaxis of conditions, such as inflammatory conditions, characterised by vasoconstriction. For example, the present invention may be particularly suited to the prophylaxis of such diseases and disorders.

The skilled person will understand that prophylaxis may be performed in a patient not suffering from the relevant disorder (i.e. such that the relevant disorder does not development). In particular, prophylaxis may be performed in a patient not suffering from the relevant disorder (i.e. such that the relevant disorder does not development) but at risk of developing (e.g. having previously had) the relevant disorder.

The skilled person will be able to identify patients (who may, in such instances, be otherwise healthy) for whom prophylaxis of a disease or disorder will be required, such as those at risk of developing the relevant disorder (e.g. patients who are have previously been treated for that disorder but do not at that time have the disorder).

The skilled person will be able to perform the determination of whether a patient has the relevant disorder or is at risk of developing the relevant disorder using techniques that are routine in that art. For example, patients not having the relevant disorder may be patients who are not experiencing associated symptoms (such as pain) at the time that prophylaxis is performed.

Thus, in a particular embodiment, the treatment or prophylaxis (e.g. the prophylaxis) as described herein is performed in a patient who is not experiencing pain (e.g. pain associated with the relevant condition as defined herein).

For example, the treatment or prophylaxis (e.g. the prophylaxis) of Raynaud's phenomenon as described herein (e.g. Raynaud's phenomenon associated with inflammation, such as Raynaud's phenomenon associated with SLE or, particularly, scleroderma) and conditions associated with Raynaud's phenomenon (such as digital ulcers) may be performed in a patient who is not experiencing pain (e.g. pain associated with the relevant Raynaud's phenomenon).

In a particular embodiment, there is provided the prophylaxis of Raynaud's phenomenon, such as that resulting from scleroderma, and conditions associated with Raynaud's phenomenon (such as digital ulcers), or pulmonary arterial hypertension (PAH).

In a more particular embodiment, there is provided the prophylaxis of Raynaud's phenomenon, such as that resulting from scleroderma.

In a further embodiment, there is provided the prophylaxis of digital ulcers, such as digital ulcers associated with scleroderma and/or Raynaud's phenomenon.

In a more particular embodiment, there is provided the prophylaxis of Raynaud's phenomenon, such as that resulting from scleroderma, in a patient who is not experiencing pain associated with the Raynaud's phenomenon.

In a further embodiment, there is provided the prophylaxis of diabetic neuropathy, diabetic retinopathy or diabetic ulcers (e.g. diabetic ulcers).

The skilled person will understand that prophylaxis of a particular disease or disorder may be performed in combination with the treatment of another disease or disorder, with the disease being treated being either unrelated to or an underlying (e.g. causative) factor of the disease or disorder the prophylaxis of which is provided. For example, the prophylaxis of digital ulcers may be achieve in combination with the treatment of Raynaud's phenomenon.

Further, the skilled person will understand that the present invention may be particularly suited to the treatment of chronic conditions, such as chronic manifestations of those diseases and disorders mentioned herein.

Thus, in particular embodiments there is provided the treatment of chronic scleroderma, chronic Reynaud's phenomenon (e.g. Reynaud's phenomenon associated with chronic scleroderma), chronic pulmonary arterial hypertension and chronic vascular complications associated with diabetes mellitus (e.g. chronic diabetic retinopathy, chronic diabetic neuropathy and chronic diabetic ulcers, such as chronic diabetic ulcers).

As used herein, the skilled person will understand that references to chronic conditions will refer to conditions that persist for an extended period of time (e.g. for at least three months, such as at least at least six months).

For the avoidance of doubt, the skilled person will understand that both the treatment of chronic conditions and the prophylaxis of conditions may be achieved through repeated administration of the mPGES-1 inhibitor, such as may be achieved by daily (e.g. once in a 24 hour period) administration of a suitable dose and/or administration in a form that allows for extended release of the active ingredient (e.g. release over a period of at least 12 hours, such as at least 24 hours) from a suitable dosage form as known to those skilled in the art.

The skilled person will understand that treatment in accordance with the present invention may further comprise (i.e. be combined with) further treatment(s) for the same condition(s). In particular, such treatment may be combined with one or more other treatment for the relevant disease or disorder (such as one or more other treatment for scleroderma, Raynaud's phenomenon, pulmonary arterial hypertension and/or vascular complications associated with diabetes mellitus, such as diabetic ulcers), as known to those skilled in the art.

For example, the skilled person will understand that:

Raynaud's phenomenon may also be treated with vasodilators such as calcium channel blockers, alpha blockers, serotonin receptor antagonists, angiotensin II receptor inhibitors, statins, locally acting nitrates or prostacyclins (such as iloprost), as known to those skilled in the art; and

pulmonary arterial hypertension may also be treated with endothelin receptor antagonists, phosphodiesterase 5 inhibitors (which agents may also be useful in the treatment of erectile dysfunction) and prostanoids.

The skilled person will understand that compounds that are mPGES-1 inhibitors may be administered in the form of pharmaceutical formulations.

In a second aspect of the invention, there is provided a pharmaceutical formulation comprising a compound that is an mPGES-1 inhibitor, or a prodrug thereof, and optionally one or more pharmaceutically acceptable excipient for use in the treatment or prophylaxis of a disease or disorder characterised by vasoconstriction.

In an alternative second aspect of the invention, there is provided a method of treating or preventing a disease or disorder characterised by vasoconstriction comprising administering to a patient in need thereof an effective amount of a pharmaceutical formulation comprising a mPGES-1 inhibitor, or a prodrug thereof, and optionally one or more pharmaceutically acceptable excipient.

The skilled person will understand that compounds that are mPGES-1 inhibitors, and pharmaceutical formulations comprising the same, will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form.

In particular, compounds that are mPGES-1 inhibitors may be administered by way of known pharmaceutical formulations (i.e. compositions suitable for use in medicine), including tablets, capsules or elixirs for oral administration, suppositories for rectal administration, sterile solutions or suspensions for parenteral or intramuscular administration, and the like.

As used herein, the skilled person will understand that references to a pharmaceutically acceptable excipient will include references to pharmaceutically acceptable adjuvants, diluents and/or carriers, as known to those skilled in the art.

For the avoidance of doubt, it is contemplated that pharmaceutical formulations as described herein may be administered in the form of tablets or capsules, e.g. time-release capsules that are taken orally, or may be in a liquid form, and may be taken orally or by injection. Such pharmaceutical formulations may also be in the form of suppositories, or, creams, gels, and foams e.g. that can be applied to the skin. In addition, they may be in the form of an inhalant that is applied nasally or via the lungs.

The skilled person will understand that mPGES-1 inhibitors as described herein, and pharmaceutical formulations comprising the same, may act systemically and/or locally (i.e. at a particular site).

Thus, pharmaceutical formulations as described herein may be administered orally, intravenously, subcutaneously, buccally, rectally, dermally, nasally, tracheally, bronchially, by any other parenteral route or via inhalation, in a pharmaceutically acceptable dosage form. Alternatively, particularly the mPGES-1 inhibitor is intended to act locally, compounds of the invention may be administered topically (in which case the pharmaceutical formulation may be a formulation for topical administration).

Depending on, for example, the potency and physical characteristics of the active ingredient (i.e. the mPGES-1 inhibitor), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

As described herein, compounds that are mPGES-1 inhibitors may also be combined with one or more other (i.e. different) therapeutic agents that are useful in the treatment of the relevant condition.

This, in a particular embodiment, the pharmaceutical formulation further comprises one or more additional therapeutic agent.

In a particular embodiment, the pharmaceutical formulation further comprises one or more additional therapeutic agent for the treatment or prophylaxis of a disease or disorder characterised by vasoconstriction (as described herein).

For example, the pharmaceutical formulation may further comprise one or more additional therapeutic agent for the treatment or prophylaxis of Raynaud's phenomenon and/or pulmonary arterial hypertension.

The skilled person will understand that pharmaceutical formulations comprising additional therapeutic agents may be presented in the form of a single formulation (i.e. a formulation comprising all of the relevant therapeutic agents) or as combination products that provide for the administration of the mPGES-1 inhibitor in conjunction with the one or more additional therapeutic agent as separate formulations (i.e. distinct formulations wherein at least one of those formulations comprises the mPGES-1 inhibitor and at least one comprises the other (additional) therapeutic agent.

As described herein, the present invention is based on the unexpected discovery that mPGES-1 inhibitors, which are known to be useful in the treatment of inflammation, will lack an inhibitory effect on the production of NO, which is known to cause vasorelaxation, and so will be particular use in the treatment of diseases and disorders characterised by vasoconstriction. This is in contrast to treatment of conditions characterised by vasoconstriction (in particular, conditions characterised by vasoconstriction having an inflammatory component) using COX inhibitors (e.g. COX-2 inhibitors), which have been found to inhibit production of NO and therefore are thought to exacerbate vasoconstriction.

Without wishing to be bound by theory, it is thought that inhibition of COX-2 leads to increased production of asymmetric dimethylarginine (ADMA), which is a naturally occurring inhibitor of eNOS. Inhibition of eNOS leads to lower levels of NO, which in turns leads to vasoconstriction. In contrast, it has now been found that mPGES-1 inhibitors surprisingly do not cause production of ADMA, thus allowing for increased activity of eNOS resulting in increased levels of NO, which in turn results in lower levels of vasoconstriction by relaxation of vessels.

### FIGURES

FIGS. 1 to 6 show results obtained from the experiments described in Examples 1 and 2 herein below.

FIG. 1 shows the effects of COX-2 inhibition using parecoxib and deletion of mPGES-1 on plasma levels of ADMA in mice (* shows p<0.05 by one-way ANOVA with Dunnett's post-hoc test).

FIG. 2 shows the effects of COX-2 inhibition using parecoxib and deletion of mPGES-1 on the expression of genes responsible for synthesis of ADMA (Prmt1) in mice (* shows p<0.05 by one sample t-test).

FIG. 3 shows the effects of COX-2 inhibition using parecoxib and deletion of mPGES-1 on the expression of genes responsible for degradation of ADMA (Agxt2) in mice (* shows p<0.05 by one sample t-test).

FIG. 4 shows that, in mPGES-1 deficient mice, deletion of mPGES-1 significantly improved the eNOS driven dilator response to acetylcholine in aorta (* shows p<0.05 by two-way ANOVA).

FIG. 5 shows that the effect observed in FIG. 4 was not mediated by an increased sensitivity of the vessels to NO, as responses to the exogenous NO donor, sodium nitroprusside, were not altered (* shows p<0.05 by two-way ANOVA).

FIG. 6 shows that the effect observed in FIG. 4 was not mediated by changes in contractility since responses to U46619 (a thromboxane mimetic) were not different between wild-type and mPGES-1 deficient mice (* shows p<0.05 by two-way ANOVA).

### EXAMPLES

The present invention will now be illustrated by way of the following examples.

General Experimental Methods

Animals

Experiments were performed on male and female mPGES-1^{-/-} and their wild-type mPGES-1^{+/+} littermate mice at 6-8 weeks of age. Mice were generated on a DBA/1J background and had a deletion in the Ptges gene by breeding heterozygous littermates and experimental animals and controls identified by genomic PCR as previously described (see Trebino, C. E. et al., Proc Natl Acad Sci USA., 100, (2003) 9044-9049). All mice were housed with a 12 h light/dark cycle in a climate-controlled environment, and were fed with standard rodent chow with water ad libitum. All mice experiments were conducted in line with EU directive 2010/63/EU and according to guidelines from the Swedish Veterinary board the Guide for the Care and Use of Laboratory Animals published by the US National Institutes of Health (NIH Publication No. 85-23, revised 1996). Studies were sanctioned by the Karolinska Institute ethics committee (dnr. N86_13 and N364_11), the Shantou University Institutional Animal Research and Use Committee and/or the Imperial College London Ethical Review Panel (PPL 70/7013 and 70/8422). Where indicated, wild-type (mPGES-1^{+/+}) mice were treated with the selective COX-2 inhibitor, parecoxib (100 mg/kg; Pfizer, USA) added to their drinking water for 5 days prior to tissue collection as we have done before (see Ahmetaj-Shala, B. et al., Circulation, 131, 633-642 (2015)).

Circulating Mediators

Mice were killed by CO₂ narcosis, and blood collected from the inferior vena cava into heparin (10 U/ml final; Leo Laboratories, UK). Plasma was separated by centrifugation for the following circulating mediators measured by immunoassay: ADMA (DLD Diagnostika, Germany), PGE₂ (Cisbio Bioassays, France).

PG Release

PG release ex vivo was measured as previously described (see Kirkby, N. S. et al., Proc Natl Acad Sci USA., 109:17597-602 (2012); and Kirkby, N. S. Et al., PLoS One, 8, e69524 (2013)). Briefly, segments of aorta (2 mm length; cleaned of peri-adventitial material), renal medulla (2×2×2 mm) or renal cortex (2×2×2 mm) were removed from experimental animals and placed in wells of microtitre plates containing DMEM media (Sigma, UK) and Ca²⁺ ionophore A23187 (30 uM; Sigma, UK) or acetylcholine (10 uM; Sigma, UK).

Gene Expression

RNA was isolated from renal medulla homogenates using a magnetic silica-bead isolation kit (Life Technologies, UK) and converted to cDNA using reverse transcriptase (Thermo Fisher Scientific, UK) with oligo(dT) primers (Life Technologies, UK). Gene expression levels were determined using TaqMan hydrolysis probes (Life Technologies, UK) recognising Prmt1 (probe ID: Mm00480133_m1 or Agxt2 (probe ID: Mm01304088_m1). Data were normalised to expression of the housekeeping genes 18S (probe ID: Mm03928990_g1) and Gapdh (probe ID: Mm99999915_g1) and relative expression compared using the comparative Ct method.

Vascular Function

Mouse aorta was isolated, cleaned of peri-advential material and divided into 2 mm rings. These were loaded into organ baths of a Malveny-Halpern wire myography (DMT, Denmark) containing Krebs buffer (composition: 120 mM NaCl; 4.7 mM KCl; 1.2 mM MgSO₄; 1.2 mM KH₂PO₄; 25 mM NaHCO₃; 0.03 mM EDTA; 5.5 mM D-glucose) at 37° C. Vessel responses were recording via a force transducer connected to a digital signal acquisition system (AD Instruments, UK). Resting tension was gradually applied to model a transmural pressure of 13.3 kPa. Vessel responsiveness was refreshed and assessed by application of three consecutive challenges with 125 mM KCl with washing with Krebs buffer in between. Concentration-response curves where then recorded in response to the thromboxane mimetic, U46619 (1-300 nM; Cayman Chemical, USA), acetylcholine (1 nM-30 μM; Sigma, UK) and sodium nitroprusside (1 nM-30 μM; Sigma UK). Responses to dilator agents (acetylcholine and sodium nitroprusside) were determined after pre-contraction of vessels with an EC₅₀ concentration of U46619 and changes in force normalised to the pre-existing tension present immediately before addition the first concentration of dilator agent.

Statistics and Data Analysis

Data were analysed using Prism 6.0 software (Graphpad software, USA) and are presented as mean±standard error for ‘n’ number of animals. Where duplicate measurements were made on tissue from the same animals, values were averaged and considered as n=1. Data were compared using Student's unpaired t-test, one sample t-test, one-way or two-away ANOVA with Dunnett's post-hoc test as indicated in individual figure legends. Differences were considered significant if p<0.05.

### Example 1

The effect on vasoconstriction of deletion of mPGES-1 was compared to that resulting from inhibition of COX-2 through investigation of their effects on levels of asymmetric dimethylarginine (ADMA), which is a naturally occurring inhibitor of eNOS.

In accordance with the general experimental methods described above, it was found that in mice:

COX-2 inhibition using parecoxib increased levels of ADMA whereas deletion of mPGES-1 had no significant effect (as shown in FIG. 1);

COX-2 inhibition using parecoxib increased expression of genes responsible for synthesis of ADMA (Prmt1) whereas deletion of mPGES-1 had no significant effect on expression of these genes (see FIG. 2); and

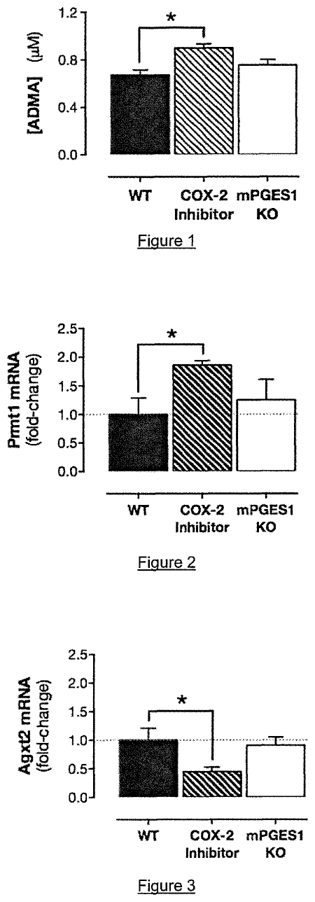

COX-2 inhibition using parecoxib increased expression of genes responsible for degradation of ADMA (Agxt2) whereas deletion of mPGES-1 again had no significant effect on expression of these genes (see FIG. 3).

### Example 2

The effect of mPGES-1 deletion on eNOS-dependent vasodilator responses induced by acetylcholine was examined.

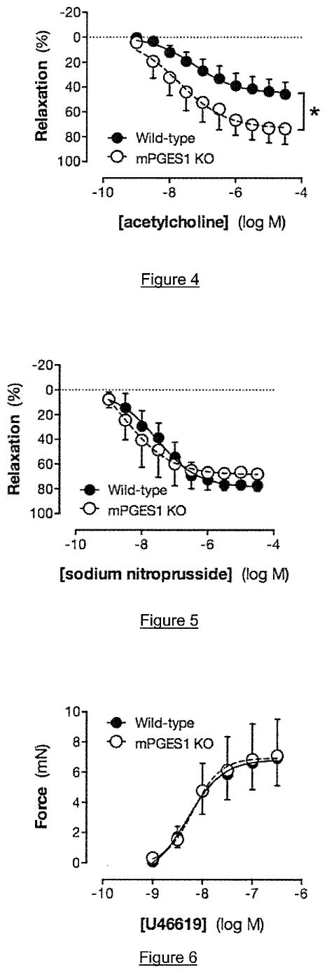

It was found that, in contrast to the effect previously seen in COX-2 deficient mice (Ahmetaj-Shala, B. et al., Circulation, 131(7), 633-42 (2015)), deletion of mPGES-1 significantly improved the eNOS driven vasodilator response to acetylcholine in aorta (as shown in FIG. 4).

Further, it was shown that this effect was not mediated by either:

an increased sensitivity of the vessels to NO, as vasodilator responses to the exogenous NO donor, sodium nitroprusside, were not altered between wild-type and mPGES-1 deficient mice (see FIG. 5); or

changes in contractility, since contraction force responses to U46619 were not different between wild-type and mPGES-1 deficient mice (see FIG. 6).

1. A method of treating pulmonary arterial hypertension (PAH) in a subject, comprising administering to a subject an effective amount of a compound represented by the following structural formula: 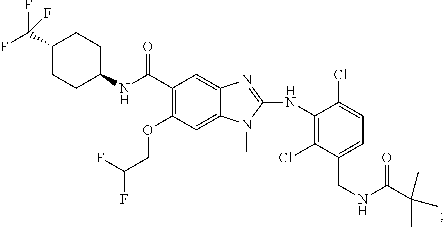 [SMILES: Cn1c(Nc2c(Cl)ccc(CNC(=O)C(C)(C)C)c2Cl)nc2cc(C(=O)N[C@H]3CC[C@H](C(F)(F)F)CC3)c(OCC(F)F)cc21]
or a pharmaceutically acceptable salt thereof.

2-17. (canceled)

18. A method of reducing the likelihood of developing pulmonary arterial hypertension (PAH) in a subject, comprising administering to a subject who is at risk of developing pulmonary arterial hypertension (PAH) an effective amount of a compound represented by the following structural formula: 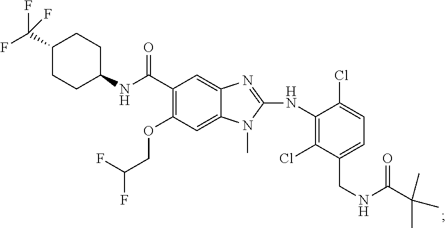 [SMILES: Cn1c(Nc2c(Cl)ccc(CNC(=O)C(C)(C)C)c2Cl)nc2cc(C(=O)N[C@H]3CC[C@H](C(F)(F)F)CC3)c(OCC(F)F)cc21]
or a pharmaceutically acceptable salt thereof, thereby reducing the likelihood of developing pulmonary arterial hypertension (PAH) in the subject.
